SEQUENCE LISTING (8 sequences)

SEQ ID NO: 1 - DNA, 29899 bp, synthetic construct
  misc_feature 1..29899: note = sequence comprising mutated NSP1
  source 1..29899: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgcagctagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 2 - DNA, 28843 bp, synthetic construct
  misc_feature 1..28843: note = sequence comprising Delta6-8
  source 1..28843: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 3 - DNA, 29899 bp, synthetic construct
  misc_feature 1..29899: note = OTS45678
  source 1..29899: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtattgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatcagaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttcaggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttgttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcattaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtcagttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattcttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcattaaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 4 - DNA, 28843 bp, synthetic construct
  misc_feature 1..28843: note = sequence comprising NSP1mut Delta6-8
  source 1..28843: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgcagctagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtattgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatcagaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttcaggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttgttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcattaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtcagttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattcttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcattaaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 5 - DNA, 28819 bp, synthetic construct
  misc_feature 1..28819: note = sequence comprising NSP1_OTS4578_delta6-8_bristoldel
  source 1..28819: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgcagctagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtattgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatcagaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttcaggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttgttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcattaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtcagttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattcttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcattaaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactatagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 6 - RNA, 28819 nt, synthetic construct
  source 1..28819: mol_type = other RNA
  misc_feature 1..28819: note = OTS 4-5-6-7-8 Nsp1 (K164A/H165A).delORF6-8.FCS
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgcagctagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtattgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatcagaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttcaggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttgttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcattaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtcagttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattcttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcattaaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactatagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 7 - DNA, 29891 bp, Severe acute respiratory syndrome coronavirus 2
  source 1..29891: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaa

SEQ ID NO: 8 - protein, 8 aa, Severe acute respiratory syndrome coronavirus 2
VNFNFNGL

(19) United States
(12) Patent Application Publication
(10) Pub. No.: US 2025/0295757 A1
(43) Pub. Date: Sep. 25, 2025

(54) ONE-TO-STOP ATTENUATED SARS-COV-2 VIRUS

(71) Applicants: UNIVERSITÄT BERN, Bern (CH); INSTITUT FÜR VIROLOGIE UND IMMUNOLOGIE (IVI), Mittelhäusern (CH)

(72) Inventors: Volker THIEL, Bern (CH); Nadine EBERT, Worb (CH); Bettina Salome TRÜEB, Bern (CH); Güliz Tuba BARUT, Bern (CH); Annika KRATZEL, Bern (CH); Jörg JORES, Köniz (CH); Fabien LABROUSSAA, Köniz (CH); Martin BEER, Neuenkirchen (DE); Donata HOFFMANN, Mesekenhagen (DE); Jacob SCHÖN, Greifswald (DE); Nico Joel HALWE, Greifswald (DE); Lorenz ULRICH, Limbach b Homburg (Saar) (DE)

(21) Appl. No.: 18/845,051

(22) PCT Filed: Mar. 28, 2023
(86) PCT No.: PCT/EP2023/058069

§ 371 Date: Sep. 9, 2024

(30) Foreign Application Priority Data
Mar. 28, 2022 (EP) 22164874.4
Oct. 12, 2022 (EP) 22201198.3

(51) Int. Cl. A61K 39/215; A61K 31/513; A61K 31/7068; A61K 39/00; A61P 37/04

(52) U.S. Cl. CPC A61K 39/215; A61K 31/513; A61K 31/7068; A61P 37/04; A61K 2039/5254; C12N 2770/20022; C12N 2770/20034; C12N 2770/20043; C12N 2770/20062; C12N 2770/20071

(57) ABSTRACT

The invention relates to a polynucleotide encoding an attenuated SARS-CoV-2 or a fragment thereof, wherein the polynucleotide comprises at least 20 one-to-stop codons. The polynucleotide may comprise further modifications and may be comprised in an attenuated SARS-CoV-2. The invention further relates to methods for production of the polynucleotide and pharmaceutical products, e.g. for medical use.

### RELATED APPLICATIONS

This application is a 35 U.S.C. § 371 filing of International Patent Application No. PCT/EP2023/058069, filed Mar. 28, 2023, which claims priority to European Patent Application Nos. 22201198.3, filed Oct. 12, 2022, and 22164874.4, filed Mar. 28, 2022, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Sep. 5, 2024, is named 757107_VOS9-023US_ST26.xml and is 215,385 bytes in size.

The invention relates to a polynucleotide encoding an attenuated SARS-CoV-2 or a fragment thereof, wherein the polynucleotide comprises at least 20 one-to-stop codons. The polynucleotide may comprise further modifications and may be comprised in an attenuated SARS-CoV-2. The invention further relates to methods for production of the polynucleotide and pharmaceutical products, e.g. for medical use.

Severe acute respiratory syndrome coronavirus 2 (SARS-CoV-2) emerged in December 2019 as the causative agent of coronavirus disease 2019 (COVID-19). The virus is highly transmissible among humans. It has spread rapidly around the world within a matter of weeks and the world is still battling with the ongoing COVID-19 pandemic.

The rapid development and availability of vaccines are crucial in combating many viruses and bacteria. The production of suitable vaccines is a multi-stage, complex process and is not always successful despite often high investments. Typically, the development of a suitable vaccine takes years. These long development times consist of a major problem, especially with regard to new emerging pathogens, or mutated , as from an epidemiological point of view it is only possible to react too late, if at all, to the emergence of new diseases. In contrast, the analysis, identification and further detection of new or heavily mutated pathogens are now possible within weeks or even days, which is a huge improvement over the last century.

In this context, viruses are of special interest, as they harbor high mutation rates causing the spread from other species to humans. Rapid spreading of these viruses makes them a major challenge for modern medicine. The usual time between the detection/identification of a newly emerging virus and the development of a vaccine is typically years. In a few cases, with sufficient prior knowledge, experimental vaccines could be provided within months. However, this period is much longer than the typical time until thousands or millions of people are infected. Such rapid spread is also a direct consequence of the high mobility of today's society.

Ideally, immediately after the identification of a new virus, a vaccine would be available in sufficient quantity and of the highest quality and would allow for a nationwide vaccination of all persons who have somehow come close to the initial outbreak site of the new virus. Furthermore, an ideal method for such a vaccine would be capable of reacting to the evolution and adaptation of the virus. Such an ideal production possibility seems utopian to the person skilled in the art today.

In the recent past, in particular, the corona pandemic has dramatically increased the relevance of developing suitable tools for vaccine production. There is unanimous agreement that the development of a vaccine against the coronavirus SARS-CoV-2 is the only proven means of containing the pandemic and the associated global crisis in the long term.

Thus, there is a need to provide means and methods that allow the production of a vaccine against the coronavirus SARS-CoV-2, in large quantities and of high quality.

The above technical problem is solved by the embodiments disclosed herein and as defined in the claims.

Accordingly, the invention relates to, inter alia, the following embodiments:
1. A polynucleotide encoding an attenuated human coronavirus or a fragment thereof, wherein the polynucleotide comprises at least 20 one-to-stop codons, wherein a one-to-stop codon is
   i) a different but synonymous codon compared to a corresponding codon in a natural human coronavirus genome or a fragment thereof; and
   ii) differs by only one nucleotide from a STOP codon.
2. The polynucleotide of embodiment 1, wherein the fragment of the polynucleotide when combined with corresponding human coronavirus parts encodes a coronavirus particle that induces an immune response after immunization of mice with 5000 PFU coronavirus particle after 15 days and an increased immune response upon challenge with WT human coronavirus after 21 days measured after 35 days.
3. A method for producing a polynucleotide of embodiment 1 or 2, the method comprising the steps of:
   a) providing the CDS of a natural human coronavirus genome, a fragment or cDNA clone thereof; and
   b) modifying the natural human coronavirus genome, the fragment or the retro-transcribed cDNA sequence of the cDNA clone, respectively,
   wherein said modification comprises replacing at least 20 codons in the natural human coronavirus genome, the fragment or the retro-transcribed cDNA sequence, by at least 20 one-to-stop codons, wherein a one-to-stop codon is
      i) a different but synonymous codon compared to a corresponding codon in the natural human coronavirus genome, the fragment or the retro-transcribed cDNA sequence; and
      ii) differs by only one nucleotide from a STOP codon.
4. The polynucleotide of embodiment 1 or 2 or the method of embodiment 3, wherein the natural human coronavirus genome or a fragment thereof is
   a) a SARS-CoV-2 sequence comprised in or consisting of a sequence as defined by SEQ ID NO: 7 or
   b) a SARS-CoV-2 sequence being 80% identical to a sequence comprised in or consisting of sequence as defined by SEQ ID NO: 7, preferably a SARS-CoV-2 sequence being 80% identical to a sequence comprised in or consisting of sequence as defined by SEQ ID NO: 7 which maintains the ability to encode one or more SARS-CoV-2 virus proteins.
5. The polynucleotide of any one of the embodiments 1, 2 or 4 or the method of embodiment 3 or 4, wherein the fragment has a minimum length of 500 nucleotides.
6. The polynucleotide of any one of the embodiments 1, 2, 4 or 5 or the method of any one of the embodiments 3 to 5, wherein the human coronavirus is SARS-CoV-2 and wherein at least one of the one-to-stop codons is comprised in a sequence part or fragment corresponding to a sequence part of ORF1ab of the natural SARS-CoV-2, a sequence part encoding a structure protein of the natural SARS-CoV-2 or a sequence part encoding an accessory protein of the natural SARS-CoV-2.
7. The polynucleotide of embodiment 6 or the method of embodiment 6, wherein at least one of the one-to-stop codons is comprised in a sequence part or fragment corresponding to a sequence part of ORF1ab of the natural SARS-CoV-2.
8. The polynucleotide of embodiment 7 or the method of embodiment 7, wherein at least one of the one-to-stop codons is comprised in a sequence part or fragment corresponding to an Nsp2 to Nsp15 encoding sequence part of the natural SARS-CoV-2 genome.
9. The polynucleotide of embodiment 8 or the method of embodiment 8, wherein at least one of the one-to-stop codons is comprised in a sequence part or fragment corresponding to an Nsp2 to Nsp7 or an Nsp13 to Nsp15 encoding sequence part of the natural SARS-CoV-2 genome.
10. The polynucleotide of embodiment 8 or 9 or the method of embodiment 8 or 9, wherein the one-to-stop codons comprise at least one one-to-stop codon having a position selected from Table 1 corresponding to a position on the natural SARS-CoV-2 genome.
11. The polynucleotide of any one of embodiments 1, 2, 4 to 10 or the method of any one of embodiments 3 to 10, wherein the amino acids encoded by the at least 20 one-to-stop codons consist of Leu, Ser, Arg and/or Gly.
12. The polynucleotide of embodiment 11 or the method of embodiment 11, wherein the amino acids encoded by the one-to-stop codons consist of Leu and/or Ser.
13. The polynucleotide of any one of embodiments 1, 2, 4 to 12 or the method of any one of embodiments 3 to 12, wherein the at least 20 one-to-stop codons are at least 50 one-to-stop codons.
14. The polynucleotide of any one of embodiments 1, 2, 4 to 13, wherein the human coronavirus is SARS-CoV-2 and wherein the polynucleotide comprises no sequence encoding a protein having an Nsp1 functionality of the natural SARS-CoV-2 or a sequence encoding a protein having a reduced Nsp1 functionality compared to the Nsp1 of a natural SARS-CoV-2, preferably wherein the polynucleotide comprises a sequence encoding a protein having a reduced Nsp1 functionality compared to the Nsp1 of a natural SARS-CoV-2, and polynucleotide comprises a mutation compared to the Nsp1 encoding sequence of natural SARS-CoV-2, wherein the mutation is K164A and/or H165A.
15. The polynucleotide of any one of embodiments 1, 2, 4 to 14, wherein the human coronavirus is SARS-CoV-2 and wherein the polynucleotide comprises no sequence encoding a protein having the functionality of a protein encoded by the ORF6 gene of the natural SARS-CoV-2 or a sequence encoding a protein having a reduced functionality of a protein encoded by the ORF6 gene of the natural SARS-CoV-2.
16. The polynucleotide of any one of embodiments 1, 2, 4 to 15, wherein the human coronavirus is SARS-CoV-2 and wherein the polynucleotide comprises no sequence encoding a protein having the functionality of a protein encoded by the ORF7a gene of the natural SARS-CoV-2 or a sequence encoding a protein having a reduced functionality of a protein encoded by the ORF7a gene of the natural SARS-CoV-2.
17. The polynucleotide of any one of embodiments 1, 2, 4 to 16, wherein the human coronavirus is SARS-CoV-2 and wherein the polynucleotide comprises no sequence encoding a protein having the functionality of a protein encoded by the ORF7b gene of the natural SARS-CoV-2 or a sequence encoding a protein having a reduced functionality of a protein encoded by the ORF7b gene of the natural SARS-CoV-2.
18. The polynucleotide of any one of embodiments 1, 2, 4 to 17, wherein the human coronavirus is SARS-CoV-2 and wherein the polynucleotide comprises no sequence encoding a protein having the functionality of a protein encoded by the ORF8 gene of the natural SARS-CoV-2 or a sequence encoding a protein having a reduced functionality of a protein encoded by the ORF8 gene of the natural SARS-CoV-2.
19. The polynucleotide of any one of embodiments 1, 2, 4 to 18, wherein the human coronavirus is SARS-CoV-2 and wherein the polynucleotide comprises a sequence part encoding a spike protein, wherein the spike protein comprises a modified or removed cleavage site compared to the cleavage site of the spike protein of the natural SARS-CoV-2.
20. The polynucleotide according to embodiment 19, wherein the polynucleotide consists of or comprises a sequence as defined SEQ ID NO: 6.
21. A vector comprising the polynucleotide of any one of the embodiments 1, 2, 4 to 20.
22. A genetically modified cell comprising the polynucleotide of any one of embodiments 1, 2, 4 to 20.
23. A method for production of an attenuated virus, the method comprising a step of culturing the genetically modified cell of embodiment 22.
24. An attenuated virus comprising the polynucleotide of any one of embodiments 1, 2, 4 to 20.
25. A pharmaceutical product comprising the vector of embodiment 21, the genetically modified cell of embodiment 22 and/or the attenuated virus of embodiment 24 for use as a medicament.
26. A pharmaceutical product comprising the vector of embodiment 21, the genetically modified cell of embodiment 22 and/or the attenuated virus of embodiment 24 for use in treatment and/or prevention of a human coronavirus infection, preferably a SARS-CoV-2 infection.
27. The pharmaceutical product for use of embodiment 25 to 26, wherein the pharmaceutical product further comprises a mutagen.
28. A method of treatment and/or prevention comprising the step of: Administering a pharmaceutical product in a therapeutically effective amount to a subject, wherein the pharmaceutical product comprises the vector of embodiment 21, the genetically modified cell of embodiment 22 and/or the attenuated virus of embodiment 24.
29. The method of embodiment 28, wherein the treatment and/or prevention is a treatment and/or prevention of a human coronavirus infection, preferably a SARS-CoV-2 infection.
30. The method of embodiment 28 or 29, wherein the method further comprises administering a mutagen in a therapeutically effective amount to a subject.
31. The pharmaceutical product for use of embodiment 27 or the method of embodiment 30, wherein the mutagen is 5-Fluorouracil or Malnupiravir.

Accordingly, in one embodiment, the invention relates to a polynucleotide encoding an attenuated human coronavirus (preferably SARS-CoV-2) or a fragment thereof, wherein the polynucleotide comprises at least 20 one-to-stop codons, wherein a one-to-stop codon is i) a different but synonymous codon compared to a corresponding codon in a natural human coronavirus genome (preferably natural SARS-CoV-2 genome) or a fragment thereof; and ii) differs by only one nucleotide from a STOP codon.

The term “polynucleotide”, as used herein, refers to a nucleic acid that includes at least 60 nucleic acid monomer units (e.g., nucleotides), typically more than 100 monomer units, and more typically greater than 200 monomer units. Polynucleotides are optionally prepared by any suitable method, including, but not limited to, isolation of an existing or natural sequence, DNA replication or amplification, reverse transcription, cloning and restriction digestion of appropriate sequences, or direct chemical synthesis by methods known in the art. The term “nucleic acid” refers to any kind of deoxyribonucleotide (e.g. DNA, cDNA, . . . ) or ribonucleotide (e.g. RNA, mRNA, . . . ) polymer or a combination of deoxyribonucleotide and ribonucleotide (e.g. DNA/RNA) polymer, in linear or circular conformation, and in either single- or double-stranded form. These terms can encompass known analogs of natural nucleotides, as well as nucleotides that are modified in the base, sugar and/or phosphate moieties (e.g. phosphorothioate backbones). In general, an analog of a particular nucleotide has the same base-pairing specificity, i.e., an analog of A will base-pair with T.

The term “attenuated human coronavirus”, as used herein, refers to a human coronavirus that, in comparison to a natural human coronavirus, provokes less and/or less severe or even no symptoms in a host organism after the host organism has been confronted (infected) with the attenuated virus. At the same time, the live attenuated virus induces an immune response of the host to the attenuated virus that is at least partially protective against a wild-type virus infection and/or at least one symptom thereof. In certain embodiments the human coronavirus is a beta coronavirus such as a beta coronavirus selected from the group consisting of: MERS-COV, SARS-CoV-1, and SARS-CoV-2, preferably SARS-CoV-2.

The term “fragment”, as used herein, refers to a sequence encoding fewer proteins and/or proteins with fewer amino acids in length than the natural human coronavirus (preferably SARS-CoV-2) genome. In some embodiments, the fragment can be used to be assembled with natural human coronavirus (preferably SARS-CoV-2) sequence parts to form a sequence that encodes an attenuated human coronavirus (preferably SARS-CoV-2). In certain embodiments, the “fragment” described herein is a plurality of sequences that together encode at least 60%, at least 70%, at least 80%, at least 90%, at least 95% or 100% of the natural human coronavirus (preferably SARS-CoV-2) genome. In certain embodiments, the fragment has a length sufficient to encode a peptide that is able to induce an immune response in a human subject.

In certain embodiments the fragment of the polynucleotide described herein when combined with corresponding human coronavirus parts encodes a coronavirus particle that induces an immune response after immunization of mice with 5000 PFU coronavirus particle after 15 days.

In certain embodiments the fragment of the polynucleotide described herein when combined with corresponding human coronavirus parts encodes a coronavirus particle that induces an immune response after immunization of mice with 5000 PFU coronavirus particle after 15 days and an increased immune response upon challenge with WT human coronavirus after 21 days measured after 35 days.

In certain embodiments the fragment of the polynucleotide described herein when combined with corresponding human coronavirus parts encodes a coronavirus particle that increases the percentage of S-Tet+ CD8+ T cells upon challenge with WT human coronavirus after 21 days measured after 26 days.

In certain embodiments the fragment of the polynucleotide described herein when combined with corresponding human coronavirus parts encodes a coronavirus particle that induces an immune response after immunization of mice with 5000 PFU coronavirus particle after 15 days and increases the percentage of S-Tet+ CD8+ T cells upon challenge with WT human coronavirus after 21 days measured after 26 days.

The “corresponding human coronavirus parts” as used herein, refers to the parts of the virus genome that is missing in the fragment. The skilled person is aware how to combine virus genome fragments. For example, coronavirus particles may be produced combining the fragment sequence with sequence parts encoding the missing proteins of the virus to a complete or substantially complete sequence that encodes the coronavirus particle. Alternatively, the coronavirus particle may be produced by a trans complementing cell line. The skilled person may use any alignment method to identify which is the closest related human corona virus and which sequence part(s) is/are corresponding human coronavirus part(s).

The “coronavirus particle” is protein-complex encoded in the combination of the fragment alone or the fragment and the corresponding coronavirus sequence parts, typically comprising a virus envelope, preferably more than half of all structural proteins, more preferably all structural proteins.

The induced and/or increased immune response is preferably measured by measurement of neutralizing antibody titers in serum of the mice in a neutralization assay, more preferably with a threshold of 20 VNT100 is considered to be an “induced immune response” (see FIGS. 18A-18C).

An increase in the percentage of S-Tet+ CD8+ T cells is preferably measured by tetramer staining (see FIGS. 18A-18C).

The skilled person is aware which animal is sensitive to the respective coronavirus and may replace the mouse with a different animal in the above described measurement setup. Depending on the type of coronavirus, the skilled person may choose for example hamsters, rats, guinea pigs, ferrets, monkeys or domestic pigs depending on the sensitivity of the WT virus instead of mice. Additionally the skilled person may make appropriate changes to the experimental setup such as the dose and timepoints. Furthermore, the animal may be genetically modified to increase sensitivity to the WT virus.

In certain embodiments, the fragment described herein has a length of at least 500, at least 1000, at least 2000, at least 3000, at least 4000, at least 5000, at least 6000, at least 7000, at least 8000, at least 9000, at least 10000, at least 15000, at least 20000 or at least 25000 nucleotides.

The term “STOP codon”, as used herein, refers to any STOP codon known in the art. In some embodiments, the STOP codon(s) is/are at least one selected from the group of UAA (RNA), UAG (RNA), UGA (RNA), TAA (DNA), TAG (DNA) and TGA (DNA).

Two codons are considered “different” herein if they differ in their nucleotides and/or nucleotide order.

Two codons are considered “synonymous” herein if they code for the same amino acid or for similar amino acids. “Similar amino acids” in the context of synonymous codons are amino acids that can be replaced and wherein the replacement does not or not substantially alter the antigenicity of the protein of which they are part. In some embodiments, synonymous codons are two codons that code for the same amino acid.

For example, the CUU codon, which codes for Leu, is replaced by the codon UUA, which also codes for Leu, but which (contrary to the CUU codon) differs by only one nucleotide from a STOP codon (i.e., from the STOP codon UAA). One-to-stop codon modifications in the polynucleotide of the invention induce differences from the wild-type (e.g., infectious) human coronavirus genome or clone by nucleotide sequence, but not by amino acid sequence (at least not before the first replication cycle).

Alternatively or complementarily, more particularly complementarily, the means of the application may involve the replacement of codon(s), which codes (code) for Thr or Ala, by codon(s) which codes (code) for Ser and differs (differ) by only one nucleotide from a STOP codon. For example, the ACA codon, which codes for Thr, may be replaced by the UCA codon, which codes for Ser, which in turn differs from the UAA STOP codon by only one nucleotide. Such codon replacement modifies the amino acid sequence of the encoded protein and therefore is selected to not (substantially) modify the antigenicity of this protein. The polynucleotide of the invention may additionally comprise further types of near to stop codons.

In some embodiments, the polynucleotide has further modifications of different nature (i.e. modifications other than one-to-stop modifications) and/or deletions that influence the amino acid sequence in the desired manner.

The term “natural human coronavirus”, as used herein, refers to any known human coronavirus preferably SARS-CoV-2 or variants derived thereof. The natural human coronavirus “genome” described herein refers to the genome itself or to a cDNA clone thereof. The natural human coronavirus genome is preferably a natural SARS-CoV-2 genome. In some embodiments, the natural SARS-CoV-2 genome described herein is the genome of a variant selected from the group of Alpha, Beta, Gamma, Delta, Omicron, Lambda, Mu, Epsilon, Zeta, Eta, Theta and Iota, preferably Omicron. In some embodiments, the natural SARS-CoV-2 genome described herein is the genome of a variant selected from the group of Alpha, Beta, Gamma, Delta, Omicron Lineage B.1.1.529, Omicron Lineage BA.2, Lambda, Mu, Epsilon, Zeta, Eta, Theta and Iota. In some embodiments, the natural SARS-CoV-2 genome described herein is the genome of a variant derived from a variant selected from the group of Delta, Omicron Lineage B.1.1.529 and Omicron Lineage BA.2. In some embodiments, the natural SARS-CoV-2 genome described herein is the genome of the Omicron Lineage. The skilled person is aware, how to retrieve the corresponding sequences. In certain embodiments, the SARS-CoV-2 genome described herein is a sequence encoding at least 50%, at least 60%, at least 70%, at least 80%, at least 90%, at least 95% or 100% of all SARS-CoV-2 proteins. In certain embodiments, the SARS-CoV-2 genome described herein is a sequence described in the GISAID dataset describing SARS-CoV-2 variants (Khare, S., et al (2021) GISAID's Role in Pandemic Response. China CDC Weekly, 3 (49): 1049-1051). Preferably the GISAID dataset describing SARS-CoV-2 variants comprising 15295201 genome sequence submissions on Mar. 28, 2023, more preferably the GISAID dataset describing SARS-CoV-2 variants on Oct. 12, 2022, even more preferably the GISAID dataset describing SARS-CoV-2 variants on Mar. 28, 2022. In some embodiments, the natural SARS-CoV-2 genome described herein is a sequence with the accession number MT108784 (SEQ ID NO: 7). The SARS-CoV-2 sequence continues to mutate. The skilled person is aware how to distinguish future mutations from other viruses. In certain embodiments, a sequence being 80%, 85%, 90%, 95%, 97%, 98%, 99% or 99.5% identical to the SARS-CoV-2 genome sequence(s) described herein is considered to be a natural SARS-CoV-2 genome, if it maintains the ability to encode one or more SARS-CoV-2 virus proteins. In some embodiments, the natural SARS-CoV-2 genome is a SARS-CoV-2 genome comprising at least one mutation selected from the group of del 69-70, RSYLTPGD246-253N, N440K, G446V, L452R, Y453F, S477G/N, E484Q, E484K, F490S, N501Y, N501S, D614G, Q677P/H, P681H and P681R. In some embodiments, the natural SARS-CoV-2 genome is a SARS-CoV-2 genome comprising at least one mutation selected from the group consisting of del 69-70, RSYLTPGD246-253N, N440K, G446V, L452R, Y453F, S477G/N, E484Q, E484K, F490S, N501Y, N501S, D614G, Q677P/H, P681H, P681R and A701V.

As such, the natural human coronavirus (preferably SARS-CoV-2) genome or fragment thereof serves as a reference sequence for the polynucleotide of the invention.

The term “corresponding” in the context of a codon in relation to the natural human coronavirus (preferably SARS-CoV-2) genome or a fragment thereof refers to the position of the codon. The skilled person is aware of how to determine a position of a corresponding codon for example using alignment techniques, for instance, using publicly available computer software such as BLAST, BLAST-2, ALIGN or Megalign (DNASTAR) software. Those skilled in the art can determine appropriate parameters for aligning sequences and determining positions, including any algorithms needed to achieve maximal alignment over the full length of the sequences being compared.

The inventors found that the human coronavirus (preferably SARS-CoV-2) virus can be attenuated by replacing codons with synonymous one-to-stop codons. These replacements do not result in changes on protein level and induce therefore an identical or similar immune response as the original virus. The presence of one-to-stop codons reduces the fitness of the virus by increasing the likelihood of a mutation to result in a STOP codon at a critical position. The inventors found that a certain number of one-to-stop codons is required to achieve a substantial attenuation of a human coronavirus (preferably SARS-CoV-2).

Accordingly, the invention is at least in part based on the finding that an attenuated human coronavirus can safely and efficiently be achieved by a polynucleotide having a certain number of one-to-stop codons.

Furthermore, the specific one-to-stop codon replacement enables more positions in the genome for specific and targeted replacements than other attenuation methods such as codon pair deoptimization. As such, the balance between attenuation and immunogenicity can be better optimized than with previous methods. Furthermore, the one-to-stop codons also allow for a targeted attenuation that can be regulated by the location and number of one-to-stop codons as well as by the presence of a mutagen.

In certain embodiments, the invention relates to a method for producing a polynucleotide of the invention, the method comprising the steps of: a) providing the CDS of a natural human coronavirus (preferably SARS-CoV-2) genome, a fragment or cDNA clone thereof; and b) modifying the natural human coronavirus (preferably SARS-CoV-2) genome, the fragment or the retro-transcribed cDNA sequence of the cDNA clone, respectively, wherein said modification comprises replacing at least 20 codons in the natural human coronavirus (preferably SARS-CoV-2) genome, the fragment or the retro-transcribed cDNA sequence, by at least 20 one-to-stop codons, wherein a one-to-stop codon is i) a different but synonymous codon compared to a corresponding codon in the natural human coronavirus (preferably SARS-CoV-2) genome, the fragment or the retro-transcribed cDNA sequence; and ii) differs by only one nucleotide from a STOP codon.

The term “CDS” of a natural human coronavirus (preferably SARS-CoV-2) genome, as used herein, refers to the coding sequence of the natural human coronavirus (preferably SARS-CoV-2) genome

The step of “modifying”, described herein, refers to altering a sequence. This alteration can be achieved by any method known in the art including resynthesis, meganucleases and Crispr.

The replacement can be achieved by removing the sequence part (e.g. the codon) from a polynucleotide and inserting the desired sequence part and/or by resynthesizing the sequence with the desired sequence part.

The inventors found that replacing certain codons in the CDS of a natural human coronavirus (preferably SARS-CoV-2) genome enables attenuation of the fitness of the encoded human coronavirus (preferably SARS-CoV-2) if enough codons are replaced.

Accordingly, the invention is at least in part based on the finding that a polynucleotide encoding an attenuated human coronavirus (preferably SARS-CoV-2) can be produced by replacing a certain number of codons with one-to-stop codons.

In certain embodiments, the invention relates to the polynucleotide of the invention or the method of the invention, wherein at least one of the one-to-stop codons is comprised in a sequence part or fragment corresponding to a sequence part of ORF1ab of the natural SARS-CoV-2, a sequence part encoding a structure protein of the natural SARS-CoV-2 or a sequence part encoding an accessory protein of the natural SARS-CoV-2.

The term “ORF1ab”, as used herein, refers to Open reading frame 1 a and/or b of the natural SARS-CoV-2.

The term “sequence part encoding an accessory gene”, as used herein, refers to accessory protein ORFs 3a, 3b, 6, 7a, 7b, 8, 9b, 9c, and/or 10.

The term “structure protein”, as used herein, refers to the SARS-CoV-2 protein S, E, M and/or N.

ORF1ab, accessory genes and structure proteins comprise information that is relevant for the fitness and reproducibility of SARS-CoV-2. The inventors found that one-to-stop codons in these sequence parts are particularly effective in attenuating SARS-CoV-2. Without being bound by theory, a mutation to a STOP codon in these areas will substantially reduce or eliminate the virus's ability to reproduce.

Accordingly, the invention is at least in part based on the finding that one-to-stop codons in the sequence parts encoding for ORF1ab, accessory genes and structural proteins are particularly effective in attenuating the SARS-CoV-2.

In certain embodiments, the invention relates to the polynucleotide of the invention or the method of the invention, wherein at least one of the one-to-stop codons is comprised in a sequence part or fragment corresponding to a sequence part of ORF1ab of the natural SARS-CoV-2.

ORF1ab is particularly relevant for the fitness and reproducibility of SARS-CoV-2. The inventors found that one-to-stop codons in these sequence parts are particularly effective in attenuating SARS-CoV-2. Without being bound by theory, a mutation to a STOP codon in this area will substantially reduce or eliminate the virus's ability to reproduce.

Accordingly, the invention is at least in part based on the finding that one-to-stop codons in the sequence parts encoding for ORF1ab are particularly effective in attenuating the SARS-CoV-2.

In certain embodiments, the invention relates to the polynucleotide of the invention or the method of the invention, wherein at least one of the one-to-stop codons is comprised in a sequence part or fragment corresponding to an Nsp2 to Nsp15 encoding sequence part of the natural SARS-CoV-2 genome.

Accordingly, the invention is at least in part based on the finding that one-to-stop codons in the sequence parts encoding for Nsp2 to Nsp15 are particularly effective in attenuating the SARS-CoV-2.

In certain embodiments, the invention relates to the polynucleotide of the invention or the method of the invention, wherein at least one of the one-to-stop codons is comprised in a sequence part or fragment corresponding to an Nsp2 to Nsp7 encoding sequence part of the natural SARS-CoV-2 genome.

In certain embodiments, the invention relates to the polynucleotide of the invention or the method of the invention, wherein at least one of the one-to-stop codons is comprised in a sequence part or fragment corresponding to an Nsp13 to Nsp15 encoding sequence part of the natural SARS-CoV-2 genome.

Accordingly, the invention is at least in part based on the finding that one-to-stop codons in certain sequence parts are particularly effective in attenuating the SARS-CoV-2.

In certain embodiments, the invention relates to the polynucleotide of the invention or the method of the invention, wherein the one-to-stop codon(s) comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 one-to-stop codon having a position selected from Table 1 corresponding to a position on the natural SARS-CoV-2 genome.

In certain embodiments, the invention relates to the polynucleotide of the invention or the method of the invention, wherein at least 10%, at least 20%, at least 30%, at least 40%, at least 50%, at least 60%, at least 70%, at least 80%, or at least 90% of the one-to-stop codons in the polynucleotide of the invention have a position selected from Table 1 corresponding to a position on the natural SARS-CoV-2 genome.

Accordingly, the invention is at least in part based on the finding that one-to-stop codons in certain positions are particularly effective in attenuating the SARS-CoV-2.

In certain embodiments, the invention relates to the polynucleotide of the invention or the method of the invention, wherein the amino acids encoded by the at least 20 one-to-stop codons consist of Leu, Ser, Arg and/or Gly.

In certain embodiments, the invention relates to the polynucleotide of the invention or the method of the invention, wherein the amino acids encoded by the one-to-stop codons consist of Leu and/or Ser.

Leu and Ser allow many combinations to design one-to-stop codons.

Accordingly, the invention is at least in part based on the finding that certain amino acids are encoded by codons that are particularly effective one-to-stop codons.

In certain embodiments, the invention relates to the polynucleotide of the invention or the method of the invention, wherein the at least 20 one-to-stop codons are at least 20, at least 25, at least 30, at least 35, at least 40, at least 45, at least 50, at least 55, at least 60; at least 65, at least 70, at least 75, at least 80, at least 85, at least 90, at least 95, at least 100, at least 105, at least 110, at least 115, at least 120, one-to-stop codons.

Accordingly, the invention is at least in part based on the finding that the attenuation of human coronavirus (preferably SARS-CoV-2) is substantial with a certain number of one-to-stop codons.

The inventors found that combining two fragments comprising one-to-stop codons particularly attenuates the encoded SARS-CoV-2 virus.

In certain embodiments, the invention relates to the polynucleotide of the invention or the method of the invention, wherein at least 20, at least 25, at least 30, at least 35, at least 40, at least 45, at least 50, at least 55 or at least 60 one-to-stop codons are comprised in one fragment.

In certain embodiments, the invention relates to the polynucleotide of the invention, wherein the polynucleotide comprises no sequence encoding a protein having an Nsp1 functionality of the natural SARS-CoV-2 or a sequence encoding a protein having a reduced Nsp1 functionality compared to the Nsp1 of the natural SARS-CoV-2.

The functions of Nsp1 are characterized (see, e.g., Min, Yuan-Qin, et al. Frontiers in microbiology (2020): 2393) and include inhibition of host mRNA translation and induction of inflammatory cytokines. Reduced or eliminated Nsp1 functionality, therefore results in reduced host (cell) stress induced by the attenuated virus. Therefore, without being bound by theory, the one-to-stop mechanism attenuates Sars-CoV-2s reproducibility and infectiousness, while the reduced Nsp1 functionality reduces the side-effects induced by the attenuated Sars-CoV-2, and increases host cell responses to infections since cellular translation is not blocked.

Accordingly, the invention is at least in part based on the finding that the combination of one-to-stop codon attenuation and reduced Nsp1 have a synergistic effect.

In certain embodiments, the invention relates to the polynucleotide of the invention, wherein the polynucleotide comprises no sequence encoding a protein having the functionality of a protein encoded by the ORF6 gene of the natural SARS-CoV-2 or a sequence encoding a protein having a reduced functionality of a protein encoded by the ORF6 gene of the natural SARS-CoV-2.

In certain embodiments, the invention relates to the polynucleotide of the invention, wherein the polynucleotide comprises no sequence encoding a protein having the functionality of a protein encoded by the ORF7a gene of the natural SARS-CoV-2 or a sequence encoding a protein having a reduced functionality of a protein encoded by the ORF7a gene of the natural SARS-CoV-2.

In certain embodiments, the invention relates to the polynucleotide of the invention, wherein the polynucleotide comprises no sequence encoding a protein having the functionality of a protein encoded by the ORF7b gene of the natural SARS-CoV-2 or a sequence encoding a protein having a reduced functionality of a protein encoded by the ORF7b gene of the natural SARS-CoV-2.

In certain embodiments, the invention relates to the polynucleotide of the invention, wherein the polynucleotide comprises no sequence encoding a protein having the functionality of a protein encoded by the ORF8 gene of the natural SARS-CoV-2 or a sequence encoding a protein having a reduced functionality of a protein encoded by the ORF8 gene of the natural SARS-CoV-2.

In certain embodiments, the invention relates to the polynucleotide of the invention, wherein the polynucleotide comprises no sequence encoding a protein having the functionality of a protein encoded by: a) the ORF8 gene and the ORF6 gene, b) the ORF8 gene and the ORF7a gene, c) the ORF8 gene and the ORF7b gene, d) the ORF6 gene and the ORF7a gene, e) the ORF6 gene and the ORF7b gene, or f) the ORF7a gene and the ORF7b gene of the natural SARS-CoV-2 or a sequence encoding a protein having a reduced functionality of a protein encoded by the respective gene combination a)-f) of the natural SARS-CoV-2.

In certain embodiments, the invention relates to the polynucleotide of the invention, wherein the polynucleotide comprises no sequence encoding a protein having the functionality of a protein encoded by: a) the ORF8 gene and the ORF6 gene and the ORF7a gene, b) the ORF8 gene and the ORF6 gene and the ORF7b gene, c) the ORF7b gene and the ORF6 gene and the ORF7a gene, or d) the ORF8 gene and the ORF7b gene and the ORF7a gene of the natural SARS-CoV-2 or a sequence encoding a protein having a reduced functionality of a protein encoded by the respective gene combination a)-d) of the natural SARS-CoV-2.

In certain embodiments, the invention relates to the polynucleotide of the invention, wherein the polynucleotide comprises no sequence encoding a protein having the functionality of a protein encoded by the ORF8 gene and the ORF6 gene and the ORF7a gene and the ORF7b gene of the natural SARS-CoV-2 or a sequence encoding a protein having a reduced functionality of a protein encoded by the ORF8 gene and the ORF6 gene and the ORF7a gene and the ORF7b gene of the natural SARS-CoV-2.

The functions of ORF6 and ORF8 are characterized and include immune-evasive mechanisms and are involved in virus host interactions. Reduced or eliminated functionality of the ORF6 gene, ORF7a gene, ORF7b gene, and/or ORF8 gene, therefore can result in reliable recognition by the immune system or impaired virus host interactions of the attenuated virus. Therefore, without being bound by theory, the one-to-stop mechanism attenuates Sars-CoV-2s reproducibility and infectiousness, while the absence or reduced functionality of the protein(s) expressed by the ORF6 gene, ORF7a gene, ORF7b gene, and ORF8 gene enhances recognition by the immune system and/or impairs virus host interactions of the attenuated SARS-CoV-2 and/or reduces the required dose of the attenuated SARS-CoV-2 to induce a certain immune response.

Accordingly, the invention is at least in part based on the finding that the combination of one-to-stop codon attenuation and ORF6, ORF7a gene, ORF7b gene, and/or ORF8 deletion or modification have a synergistic effect.

In certain embodiments, the invention relates to the polynucleotide of the invention, wherein the polynucleotide comprises a sequence part encoding a spike protein, wherein the spike protein comprises a modified or removed cleavage site compared to the cleavage site of the spike protein of the natural SARS-CoV-2.

The inventors found, that upon production of the attenuated SARS-CoV-2, the virus tends to mutate in the host cells and modify the cleavage site or remove the cleavage site in the spike protein. By starting with a sequence comprising a modified or removed cleavage site in the starting sequence, the sequence gets replicated more uniformly and/or more efficiently.

The inventors found, that upon infection with the attenuated SARS-CoV-2, virus transmission to co-housed animals was absent or reduced when an attenuated SARS-CoV-2 was used that lacks the cleavage site in the spike protein.

The inventors found that replication of an attenuated SARS-CoV-2 lacking the cleavage site in the spike protein was still efficient in mucosal tissues of the upper respiratory tract, while replication in the lungs was reduced.

Accordingly, the invention is at least in part based on the finding that modifying or removing the cleavage site of the spike protein improves the production of an attenuated SARS-CoV-2 virus, reduces transmission, and reduces replication in the lower respiratory tract.

In certain embodiments, the invention relates to a polynucleotide according to the invention, wherein the polynucleotide consists of or comprises a sequence as defined SEQ ID NO: 6.

In certain embodiments, the invention relates to a vector comprising the polynucleotide of the invention.

The term “vector”, as used herein, refers to a nucleic acid molecule that is designed for being incorporated and expressed by a cell or for transfer between different host cells. A cloning or expression vector may comprise elements, for example, regulatory and/or post-transcriptional regulatory elements and a promoter. A vector may include sequences that allow direct autonomous replication in a cell or may include sequences sufficient to allow integration into host cell DNA. In some embodiments, the vector described herein is a vector selected from the group of plasmids (e.g., DNA plasmids or RNA plasmids), shuttle vectors, transposons, cosmids, artificial chromosomes (e.g. bacterial, yeast, human), and viral vectors.

In some embodiments, the vector described herein is used in combination with at least one transfection enhancer, e.g., a transfection enhancer selected from the group of oligonucleotides, lipoplexes, polymersomes, polyplexes, dendrimers, inorganic nanoparticles and cell-penetrating peptides.

Transduction of host cells by the vector of the invention can be achieved by stable or transient transduction (see, e.g., Stepanenko, A. A., and Heng, H. H., 2017, Mutation Research/Reviews in Mutation Research, 773, 91-103).

In certain embodiments, the invention relates to a genetically modified cell comprising the polynucleotide of the invention.

The term “genetically modified cell”, as used herein, refers to a cell modified by means of genetic engineering. The term as used herein “engineered” and other grammatical forms thereof may refer to one or more changes of nucleic acids, such as nucleic acids within the genome of an organism.

In some embodiments, the genetically modified cell described herein is a host cell for the production of an attenuated human coronavirus (preferably SARS-CoV-2) or for amplification of the polynucleotide of the invention. The term “host cell”, as used herein, refers to a cell into which exogenous nucleic acid has been introduced, including the progeny of such a cell. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein.

In some the host cell described herein comprises at least one cell type selected from the group of Vero, VeroE6, VeroE6-TMPRSS2, A549-hACE2, HEK293, MDCK, Chinese hamster ovary (CHO), BHK-21, SF9, MRC 5, Per.C6, PMK, and WI-38.

In some embodiments, the genetically modified cell is a cell for use in cell therapy.

In certain embodiments, the invention relates to a method for production of an attenuated virus, the method comprising a step of culturing the genetically modified cell of the invention.

Methods for culturing cells are known in the art (see, e.g., Celis, Julio E., ed. Cell biology: a laboratory handbook. Vol. 1. Elsevier, 2005).

In certain embodiments, the invention relates to an attenuated virus comprising the polynucleotide of the invention.

In some embodiments, the attenuated virus described herein further comprises structural proteins of SARS-CoV-2, preferably all structural proteins of SARS-CoV-2.

In certain embodiments, the invention relates to a pharmaceutical product comprising the vector of the invention, the genetically modified cell of the invention and/or the attenuated virus of the invention.

In certain embodiments, the invention relates to a pharmaceutical product comprising the vector of the invention, the genetically modified cell of the invention and/or the attenuated virus of the invention for use as a medicament.

The term “pharmaceutical product”, as used herein, refers to a preparation which is in such form as to permit the biological activity of an active ingredient contained therein to be effective, and which contains no additional components which are unacceptably toxic to a subject to which the formulation would be administered.

The terms “use as a medicament” or “treatment” (and grammatical variations thereof such as “treat” or “treating”), as used herein, refer to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

In some embodiments, the pharmaceutical product comprises auxiliary substances like carriers and/or adjuvants, e.g., for enhancing an immune response of a patient. In some embodiments, the adjuvants described herein are at least one selected from the group of potassium alum; aluminum hydroxide; aluminum phosphate; calcium phosphate hydroxide; aluminum hydroxyphosphate sulfate; paraffin oil; propolis; killed bacteria of the species Bordetella pertussis or Mycobacterium bovis; plant saponins from Quillaja, soybean, and/or Polygala senega; cytokines IL-1, IL-2, and/or IL-12; as well as Freund's complete adjuvant. In some embodiments, the pharmaceutical product described herein comprises the vector of the invention and vector stabilizers and/or nanoparticles such as LNPs.

The dose is chosen such that the pharmaceutical product is well tolerated by the patient but evokes an immune response that gives desired medical effect, such as protection against infection or against a severe progression of an infection. In an embodiment, the dose is the lowest protective dose, the highest tolerable dose or lies between the lowest protective dose and the highest tolerable dose.

In some embodiments, the pharmaceutical product comprises the vector of the invention in a dose of at least 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, or more, vector genomes per kilogram (vg/kg) of the weight of the subject.

In some embodiments, the pharmaceutical product comprises the attenuated virus of the invention in a dose between 1*10³ and 1*10⁸ plaque-forming units (PFU) or focus-forming units (FFU), in particular between 1*10⁴ and 1*10⁷ PFU or FFU, in particular between 1*10⁵ and 1*10⁶ PFU or FFU, of the attenuated virus.

Various factors can influence the dose used for a particular application. For example, the frequency of administration, duration of treatment, preventive or therapeutic purpose, the use of multiple treatment agents, route of administration, previous therapy, the patient's clinical history, the discretion of the attending physician and severity of the disease, disorder and/or condition may influence the required dose to be administered.

As with the dose, various factors can influence the actual frequency of administration used for a particular application. For example, the dose, duration of treatment, use of multiple treatment agents, route of administration, and severity of the disease, disorder and/or condition may require an increase or decrease in administration frequency.

In some cases, an effective duration for administering the pharmaceutical product of the invention (and any additional therapeutic agent) can be any duration that reduces the severity, or occurrence, of symptoms of the disease, disorder and/or condition to be treated without producing significant toxicity to the subject. Multiple factors can influence the actual effective duration used for a particular treatment. For example, an effective duration can vary with the frequency of administration, effective amount, use of multiple treatment agents, route of administration, and severity of the disease, disorder and/or condition being treated.

In some embodiments, the pharmaceutical product is administered to the patient at once. In some embodiments, the pharmaceutical product is administered to the patient at least two times, wherein the second administration is separated from the first administration by a first time period. In this context, the first time period lies in a range of from 2 weeks to 36 months, in particular of from 3 weeks to 30 months, in particular of from 4 weeks to 24 months, in particular of from 5 weeks to 21 months, in particular of from 6 weeks to 18 months, in particular of from 7 weeks to 15 months, in particular of from 8 weeks to 12 months, in particular of from 9 weeks to 10 months, in particular of from 10 weeks to 8 months, in particular of from 12 weeks to 6 months, in particular of from 13 weeks to 4 months.

In an embodiment, the pharmaceutical product is administered to the patient temporally offset to administering a different vaccine (such as, e.g., a vector-based vaccine, an mRNA-based vaccine, a protein-based vaccine) to the patient, i.e., after or before vaccinating the patient with the different vaccine. In this context, the administration of the pharmaceutical product is offset to the administration of the different vaccine by a second time period. In this context, the second time period lies in a range of from 2 weeks to 36 months, in particular of from 3 weeks to 30 months, in particular of from 4 weeks to 24 months, in particular of from 5 weeks to 21 months, in particular of from 6 weeks to 18 months, in particular of from 7 weeks to 15 months, in particular of from 8 weeks to 12 months, in particular of from 9 weeks to 10 months, in particular of from 10 weeks to 8 months, in particular of from 12 weeks to 6 months, in particular of from 13 weeks to 4 months.

In certain embodiments, the invention relates to a pharmaceutical product comprising the vector of the invention, the genetically modified cell of the invention and/or the attenuated virus of the invention for use in treatment and/or prevention of a human coronavirus (preferably SARS-CoV-2) infection.

In certain embodiments, the invention relates to a pharmaceutical product comprising the vector of the invention, the genetically modified cell of the invention and/or the attenuated virus of the invention for use in treatment and/or prevention of a symptom of human coronavirus (preferably SARS-CoV-2) infection.

Symptoms of a SARS-CoV-2 infection include, without limitation, cough, fatigue, difficulty breathing, chills, joint or muscle pain, expectoration, sputum production, dyspnoea, myalgia, arthralgia or sore throat, headache, nausea, vomiting, diarrhea, sinus pain, stuffy nose, reduced or altered sense of smell or taste, lack of appetite, loss of weight, stomach pain, conjunctivitis, skin rash, lymphoma, apathy, and somnolence, preferably fever, cough, fatigue, difficulty breathing, chills, joint or muscle pain, expectoration, sputum production, dyspnoea, myalgia, arthralgia, sore throat, headache, nausea, vomiting, diarrhea, sinus pain, stuffy nose and reduced or altered sense of smell or taste.

The inventors found that the means and methods described herein can be used to induce an immune response that is useful in the treatment and/or prevention of a human coronavirus (preferably SARS-CoV-2) infection. In certain embodiments, the pharmaceutical product described herein is a vaccine and/or a vaccine booster.

In certain embodiments, the invention relates to the pharmaceutical product for use of the invention, wherein the pharmaceutical product further comprises a mutagen.

A method of treatment and/or prevention comprising the step of: Administering a pharmaceutical product in a therapeutically effective amount to a subject, wherein the pharmaceutical product comprises the vector of the invention, the genetically modified cell of the invention and/or the attenuated virus of the invention.

The method of treatment and/or prevention of the invention, wherein the treatment and/or prevention is a treatment and/or prevention of a human coronavirus (preferably SARS-CoV-2) infection.

The method for treatment and/or prevention of the invention, wherein the method further comprises administering a mutagen in a therapeutically effective amount to a subject.

In certain embodiments, the invention relates to a combination of a mutagen with a polynucleotide encoding an attenuated virus or a fragment thereof, wherein the polynucleotide comprises at least 20 one-to-stop codons, wherein a one-to-stop codon is i) a different but synonymous codon compared to a corresponding codon in a natural virus genome or a fragment thereof; and ii) differs by only one nucleotide from a STOP codon. The attenuated virus is preferably a human coronavirus, more preferably a beta coronavirus, even more preferably SARS-CoV-2.

The combination may be administered simultaneously or sequentially. As such the administration of the mutagen described herein can occur prior to, simultaneously, and/or following, administration of the polynucleotide described herein. In certain embodiments, the combination described herein is in a composition for simultaneous administration or in several separate compositions for simultaneous or sequential administration. The mutagen and the polynucleotide described herein can be administered by the same administration route (e.g., parenteral) or by different administration routes (e.g. oral administration for the mutagen und parenteral administration for the polynucleotide described herein). In a preferred embodiment, the mutagen described herein is administered repeatedly, preferably more often than the polynucleotide described herein.

The attenuation encoded in the polynucleotide can therefore be enhanced by the mutagen. The mutagen may therefore be used in subjects where a non-typical (e.g. stronger side effects, more in vivo proliferation than usual) immune response is expected or observed. In certain embodiments, the combination of the mutagen and the polynucleotide described herein is administered to a subject with an altered immune system function. The immune system function alteration can be induced, without limitation by a disease or disorder (such as infection, autoimmune disease, cancer, immunodeficiency (acquired or congenital) or obesity) and/or by an immunomodulatory treatment (e.g., DMARDs, IMiDs and/or oncological treatment).

Alternatively, the immune response to an attenuated virus can be measured and when reaching a certain threshold may be stopped or tampered by administration of the mutagen.

The mutagen may also be equivalently combined with the attenuated virus of the invention, the host cell of the invention, or the vector of the invention instead of the polynucleotide described herein. In certain embodiments, the mutagen described herein is an RNA-nucleotide analog. In certain embodiments, the mutagen described herein is 5-Fluorouracil or Malnupiravir

As such, the invention is at least in part based on the finding, that the attenuation of a one-to-stop attenuated virus can be regulated by a mutagen.

All embodiments of the polynucleotide can be combined in any desired way and can be transferred either individually or in any arbitrary combination to the attenuated human coronavirus (preferably SARS-CoV-2), to the pharmaceutical composition, its use, to the method of treatment, to the vector, to the host cell, and to the method of producing a virus.

“a,” “an,” and “the” are used herein to refer to one or to more than one (i.e., to at least one, or to one or more) of the grammatical object of the article. “or” should be understood to mean either one, both, or any combination thereof of the alternatives. “and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF FIGURES

FIG. 1: Schematic illustration of generation of recombinant SARS-CoV-2 using “transformation-associated recombination” (TAR) cloning is yeast, subsequent generation of in vitro transcribed RNA resembling the recombinant SARS-CoV-2 RNA genome, and subsequent assessment of the virus phenotype.

FIG. 2: SARS-CoV-2 genome; Modular “One-to-stop” (OTS) cloning strategy

FIGS. 3A-3B: SARS-CoV-2-OTS replication in primary airway epithelial cultures. Virus titer (Tissue culture infectious dose 50%; TCID50) was determined at 0 (inoculum), 1, 24, 48, 72, 96 hours post-infection in apical washes. FIG. 3A: OTS-Clones: 96-hour kinetics on hNEC at 33° C.; FIG. 3B: OTS-Clones: 96-hour kinetics on hNEC at 37° C.

FIGS. 4A-4E: OTS8, OTS4-5 were assessed for attenuation:

FIG. 4A: body weight, FIG. 4B: clinical score, FIG. 4C: Histopathological score, FIG. 4D: viral copies, FIG. 4E: Virus titer

FIGS. 5A-5E: OTS2, OTS7, and OTS7-8 were assessed for attenuation:

FIG. 5A: body weight, FIG. 5B: clinical score, FIG. 5C: Histopathological score, FIG. 5D: viral copies, FIG. 5E: Virus titer

FIGS. 6A-6M: OTS4-5 and OTS7-8 attenuation and protection. Mice were immunized with OTS4-5, OTS-7-8. At day 7 half of the mice were euthanized for analysis. Challenge with pathogenic wild-type virus was done at 21 days post immunization.

FIG. 6A: Pre-challenge survival FIG. 6B: Post-challenge survival (note of FIG. 6A and FIG. 6B that at day 7 post immunization 50% of mice were euthanized for analysis), FIG. 6C: Pre-challenge weight FIG. 6D: Post-challenge weight FIG. 6E: Pre-challenge score, FIG. 6F: Post-challenge score FIG. 6G: viral copies 7 days post immunization: Animals with high clinical score and body weight loss FIG. 6H: viral copies at day 26 (day 5 post challenge), FIG. 6I: viral copies at day 35 (14 days post challenge), FIG. 6J: Pre-challenge viral copies oropharyngeal swabs, FIG. 6K: Post-challenge viral copies oropharyngeal swabs, FIG. 6L: viral titer at 5 days post challenge FIG. 6M: viral titer at 14 days post challenge

FIGS. 7A-7C: OTS4-5 and OTS7-8 attenuation and protection

FIG. 7A: Neutralizing Antibody Assay against Wuhan WT: neutralizing antibody titers, FIG. 7B: Spike-specific CD8+ T cells: T cell responses, FIG. 7C: Histopathological score,

FIGS. 8A-8J: OTS4-5 and OTS4-5-7-8 were assessed for attenuation:

FIG. 8A: survival, FIG. 8B: clinical score, FIG. 8C: body weight, FIG. 8D: Swabs, FIG. 8E-8G: RNA, FIG. 8H-8J: PFU

FIG. 9: Construct overview

FIGS. 10A-10B: Naive Syrian hamsters (also ferr/mice) with one-to-stop 4-5/7-8 construct

P=Nasal washing

FIG. 10A:

intra nasal inoculation: 5000 PFU/hamster, OTS4-5/7-8 inoculated N=10, WT inoculated control N=4, OTS4-5/7-8 contact N=4; Co-housing: Co-housing of the contact groups; Necropsy 1: Necropsy of half of inoculated and control group; Necropsy 2: Necropsy of 5 inoculated and contacts

FIG. 10B:

intra nasal inoculation: 5000 PFU/hamster, OTS4-5/7-8 inoculated N=8, OTS4-5/7-8 contact N=3; Challenge: Challenge of inoculated and N=4 naive control with WT 5000 PFU/hamster and co-housing of the contact groups; Necropsy: Necropsy of inoculated and contacts. Can apply for 5 dpc necropsy.

FIG. 11A: Hamster survival; FIG. 11B: Relative body weight

FIG. 12: genome copies

FIG. 13: Humoral immune response (RBD-ELISA-Data) of OTS inoculated and direct contact animals. FCS deletion prevent transmission of final OTS to naïve contact animals.

FIG. 14: Tissue specific gene copies 5 days post inoculation with WT or final OTS.

FIG. 15: Humoral immune response (RBD-ELISA-Data) at 14 dpc. Final OTS (SEQ ID NO: 6) prevent transmission of challenge virus to naïve contact animals.

FIG. 16A: 5-FU:

Cells: VeroET cells; Pre-treatment for 30 min; Infection with MOI: 0.1 for 1 h with ID3 and ID194; Remove inoculum and add DMEM+drug in concentration ranging from 40-280 uM; Harvesting and TCID50 24h pi

FIG. 16B: Molnupiravir:

Cells: VeroET cells; Pre-treatment for 30 min; Infection with MOI: 0.1 for 1 h with ID3 and ID194; Remove inoculum and add DMEM+drug in concentration ranging from 0.1-10 uM; Harvesting and TCID50 24h pi

FIG. 17

Human bronchial epithelial cell (hBEC) cultures were infected with SARS-CoV-2 WT, as well as SARS-CoV-2 with OTS codons in either Fragment 2, 7 or 8 (OTS2, 7, 8). Viral titers are shown until 96 hours post infection in TCID50/ml. OTS2 is significantly attenuated at 72 and 96 hpi.

FIGS. 18A-18C

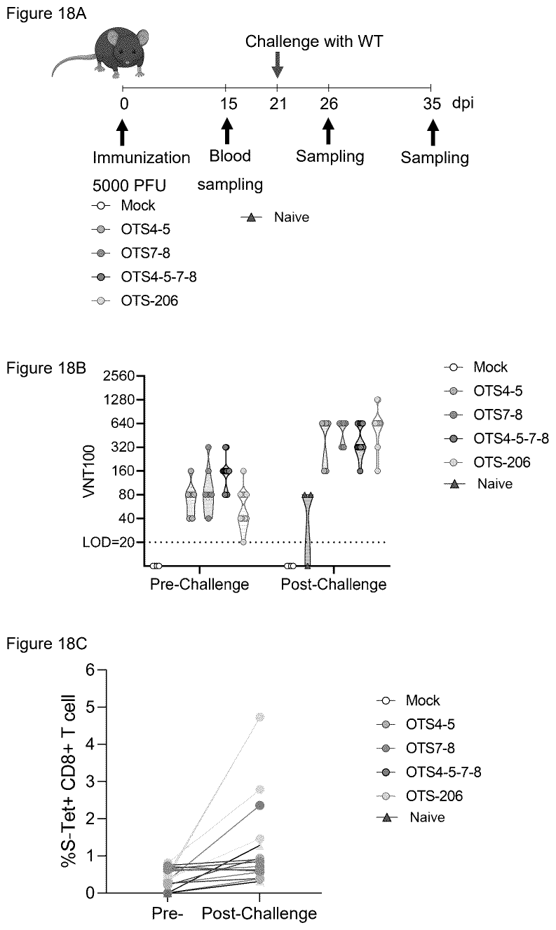

Assessment of immune responses. FIG. 18A: Experimental design to assess virus-specific immune responses. Mice were immunized by infection with attenuated SARS-CoV-2 OTS4-5, OTS7-8, OTS4-5-7-8, OTS-206 or were mock infected. Challenge with wt SARS-CoV-2 was performed 21 days later. FIG. 18B: Determination of SARS-CoV-2 neutralizing antibody titers in serum obtained from mice at days 15 (pre-challenge) and days 35 (post-challenge) by virus neutralization assay. FIG. 18C: Determination of SARS-CoV-2-specific CD8+T-cell responses at days 15 (pre-challenge) and days 26 (post-challenge) by tetramer staining (H-2K(b) SARS-CoV-2 spike epitope 539-546 (VNFNFNGL) SEQ ID NO:8).

### EXAMPLES

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention.

### Example 1

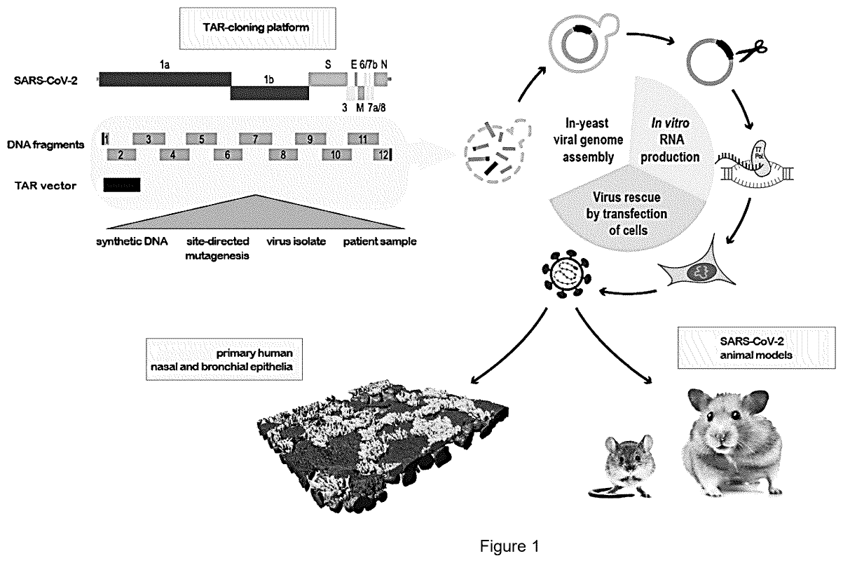

Generation of recombinant SARS-CoV-2 was done using “transformation-associated recombination” (TAR) cloning is yeast (12 overlapping DNA fragments spanning the entire SASRS-CoV-2 genome), subsequent generation of in vitro transcribed RNA resembling the recombinant SARS-CoV-2 RNA genome, and rescue of infectious recombinant viruses following transfection of in vitro transcribed RNA into BHK-SARS-N cells (Thi Nhu Thao, Tran, et al., 2020, Nature 582.7813:561-565; and FIG. 1).

Recombinant viruses were characterized in vitro in VeroE6 and VeroE6-TMPRSS2 cells, and primary human airway epithelial cultures. In vivo viruses were assessed in various animal models including K18-hACE2-mice, hACE2-KI-mice and Syrian hamsters (FIG. 1)

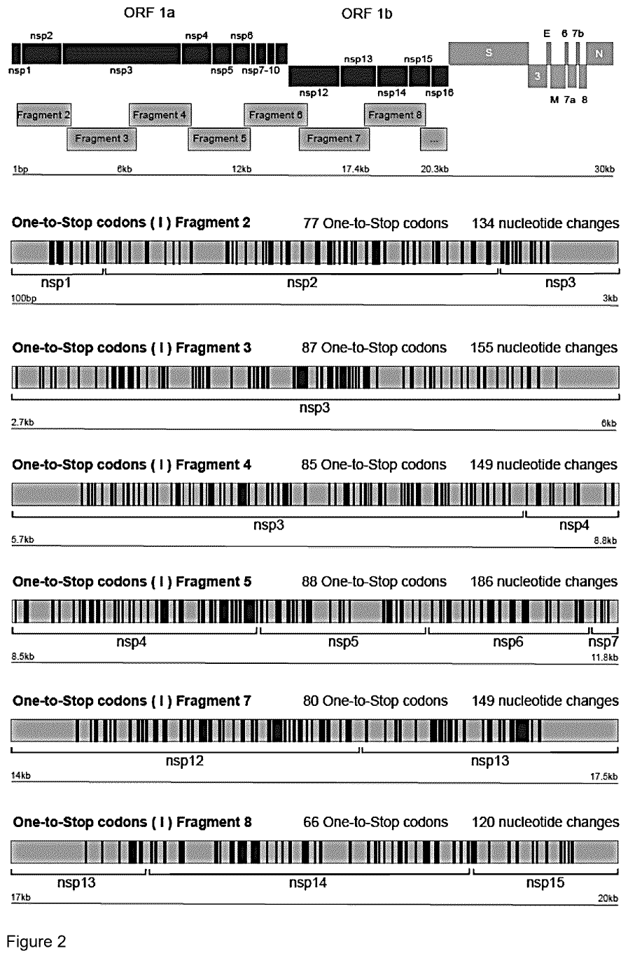

Cloning: A set of synthetic DNA fragments were designed to contain an enriched number of OTS codons encoding for Leu or Ser (see Table 1). Fragments 2-5,7-8 (see FIG. 2) were selected since these encode for the viral replicase gene product and increased appearance of stop codons in this region of the genome were considered to be most effective in generating attenuated viruses.

The constructs were cloned and analyzed further.

SARS-CoV-2-OTS replication in primary airway epithelial cultures:

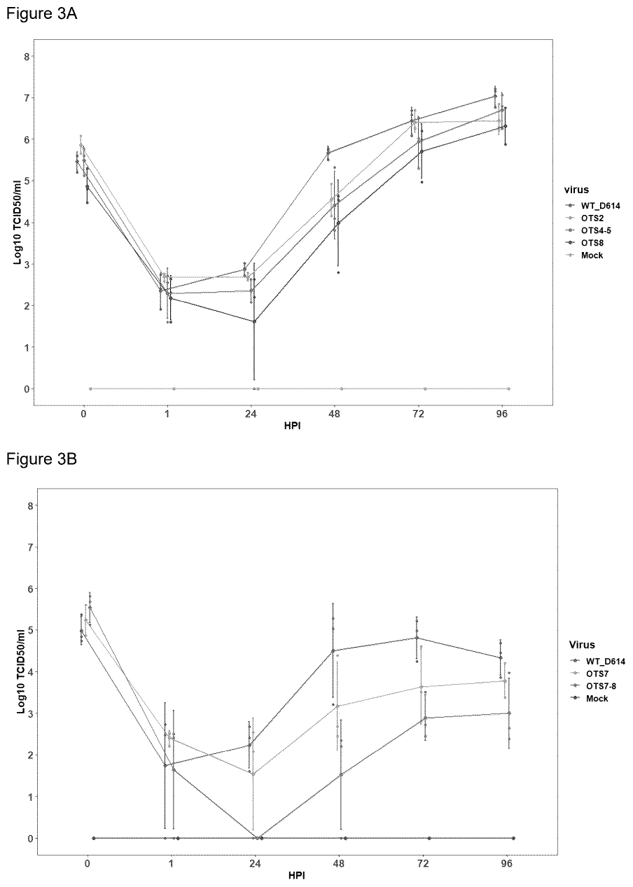

Virus titer was determined at 0 (inoculum), 1, 24, 48, 72, 96 hours post infection in apical washes. (FIGS. 3A-3B)

Assessment of Attenuation and Protection in kI8-hACE2-Mice:

Based on the replication kinetics determined in primary human epithelial cultures the following experiments were conducted in vivo.

### Assessment of Attenuation:

K18-hACE2-mice were infected intranasally with 5000 PFU. Oropharyngeal swabs were taken daily. Organs were taken at days 2 and 5/6 post infection. Viral RNA was quantified by qRT-PCR and viral titers were determined by plaque assay (to determine PFUs). Clinical scores and body weight were determined daily.

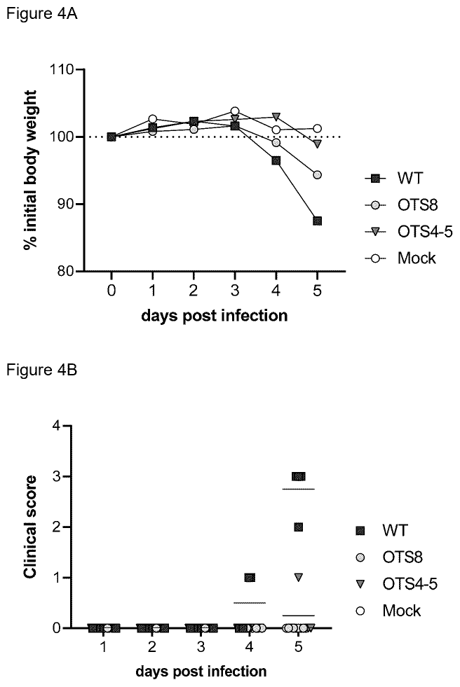

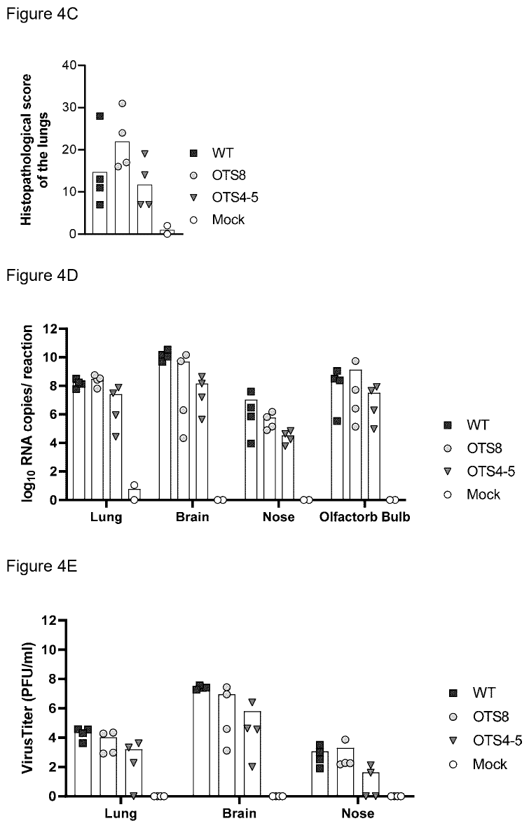

OTS8, OTS4-5 were assessed for attenuation (FIGS. 4A-4E).

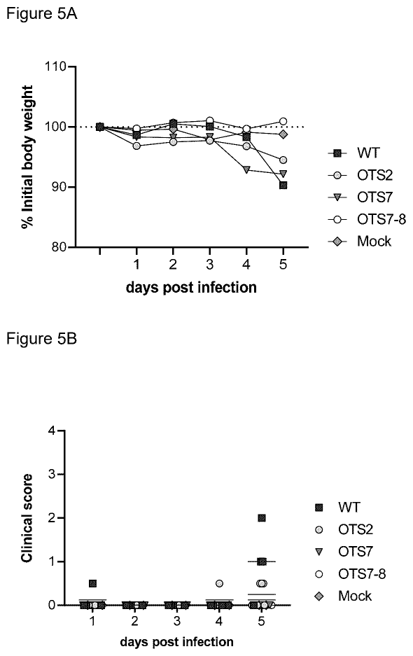

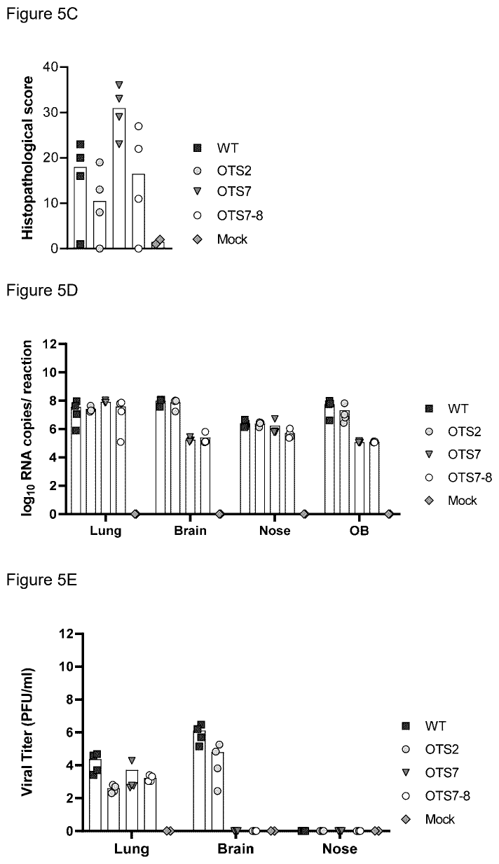

OTS2, OTS7, OTS7-8 were assessed for attenuation (FIGS. 5A-5E).

### Assessment of Attenuation and Protection:

K18-hACE2-mice were infected intranasally with 5000 PFU. Oropharyngeal swabs were taken daily. Organs were taken at days 2 and 5/6 post infection. Viral RNA was quantified by qRT-PCR and viral titers were determined by plaque assay (to determine PFUs). Clinical scores and body weight were determined daily.

Challenge: >21 days post infection mice were challenged with wt SARS-CoV-2 (5000 PFU) and monitored for additional 15 days. Body weight and clinical scores were detected daily. Viral RNA load, virus titers were determined at 5 and 14/15 days post challenge. Swabs were taken 3-4 times per week. Antibody titers and CD8 T-cell responses were determined at the indicated time points.

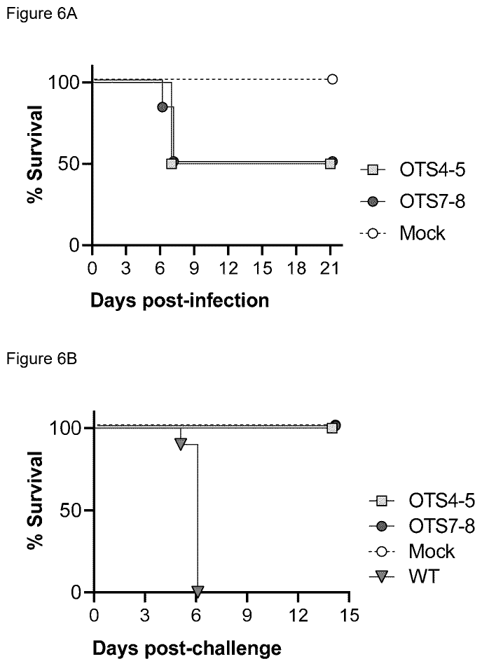

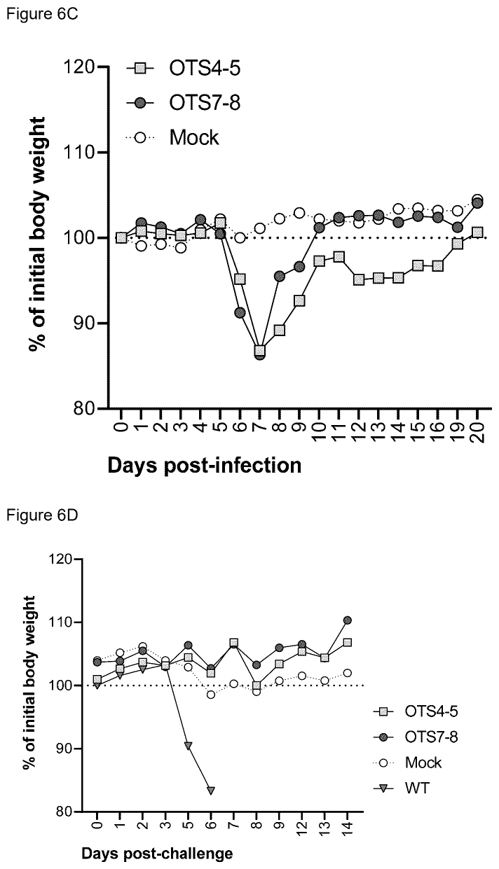

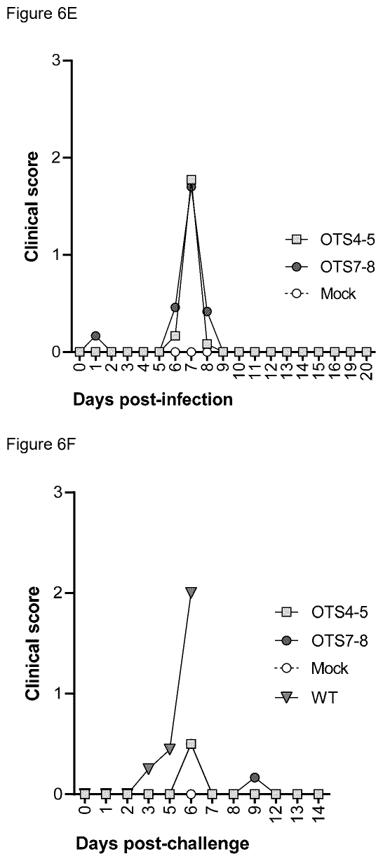

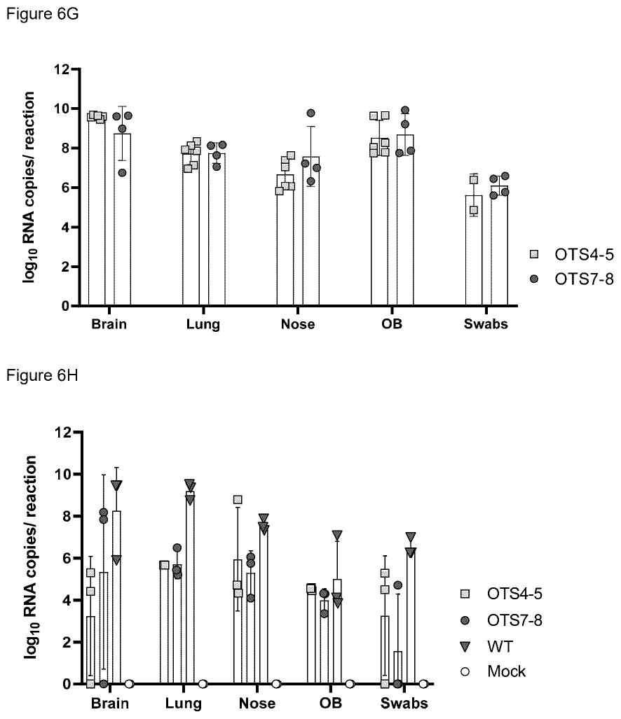

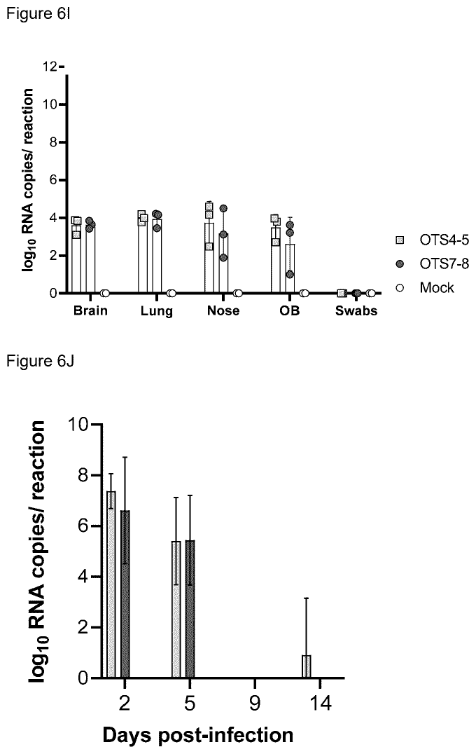

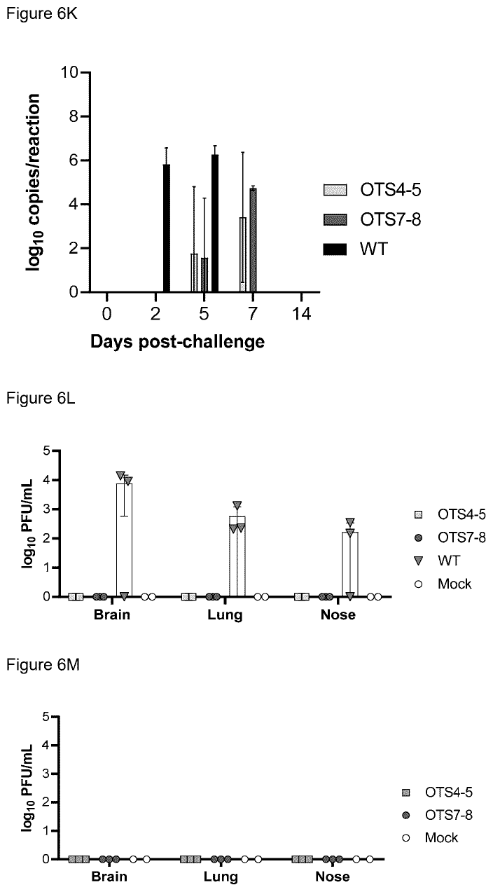

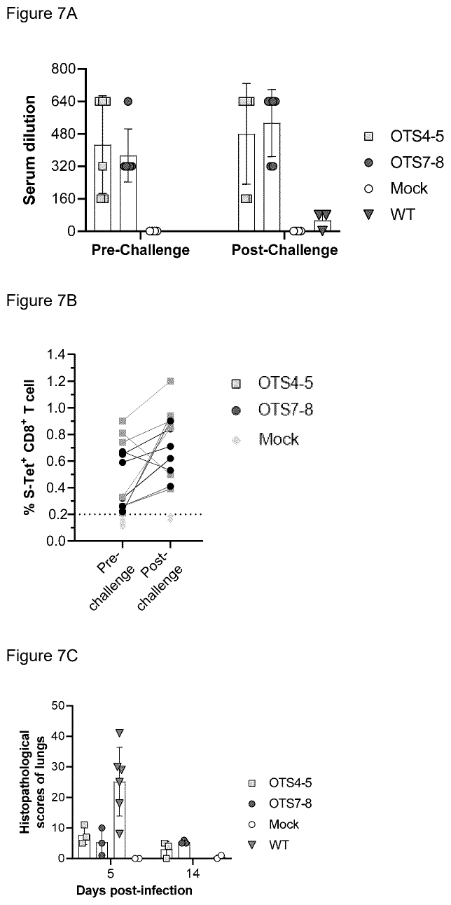

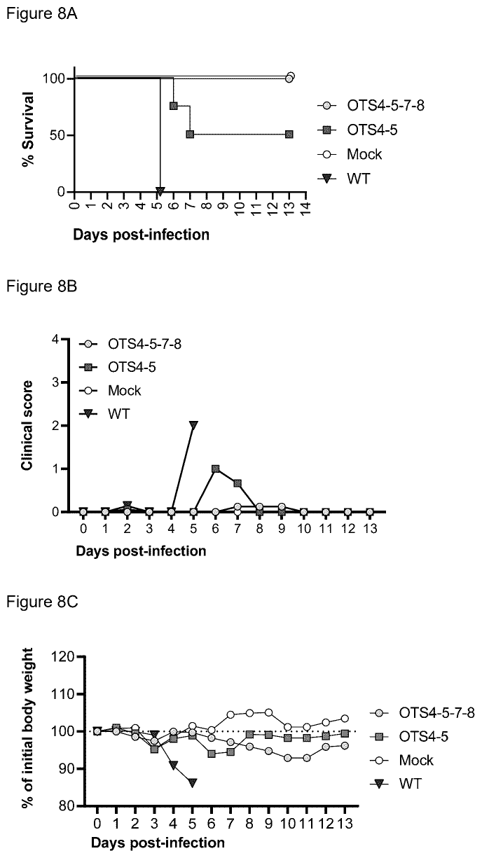

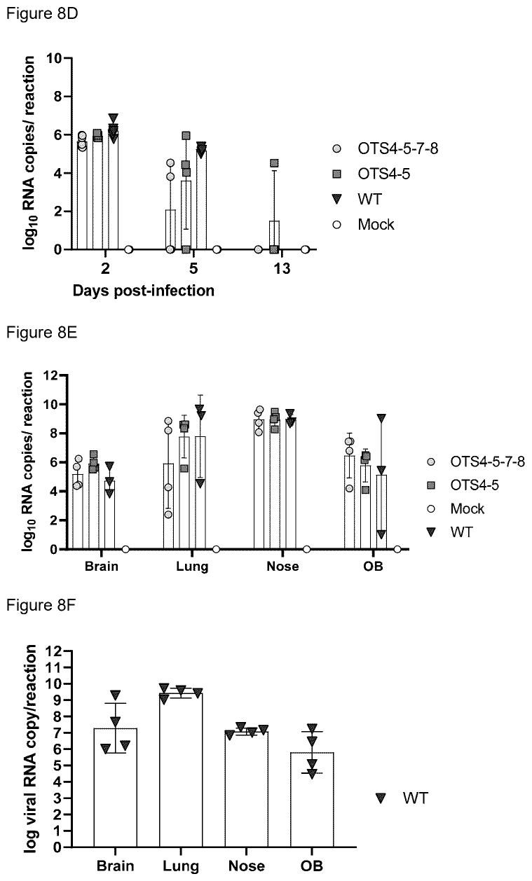

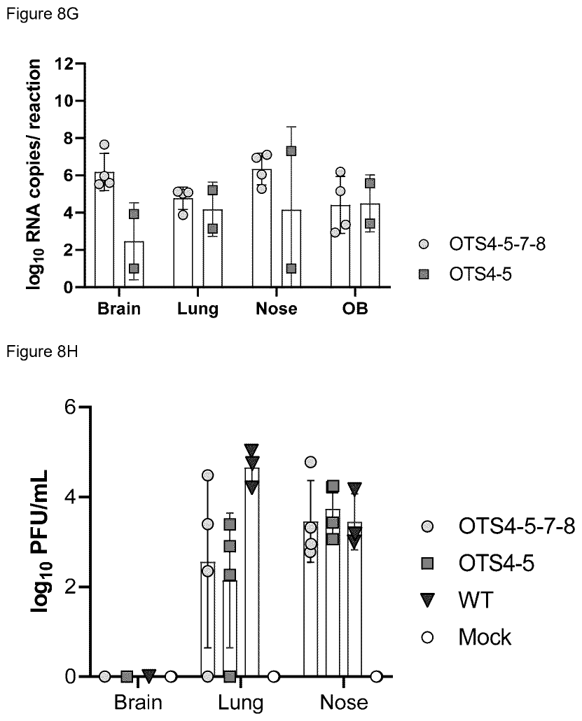

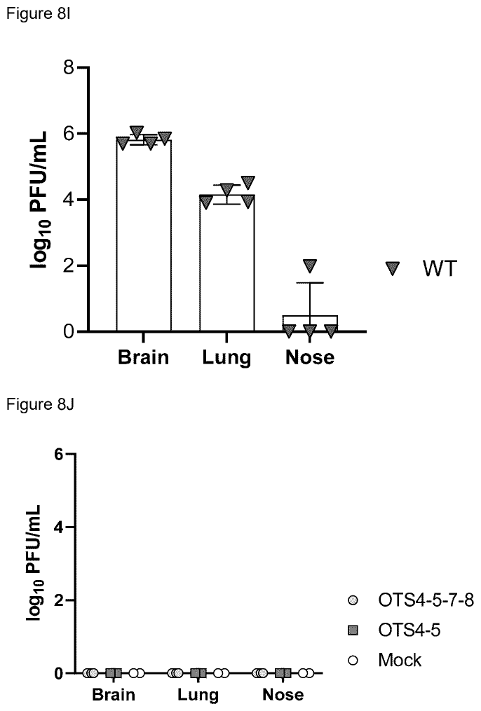

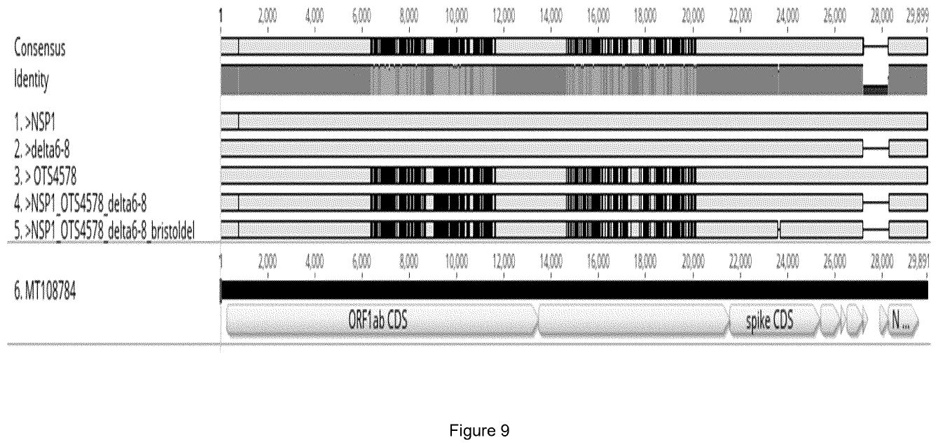

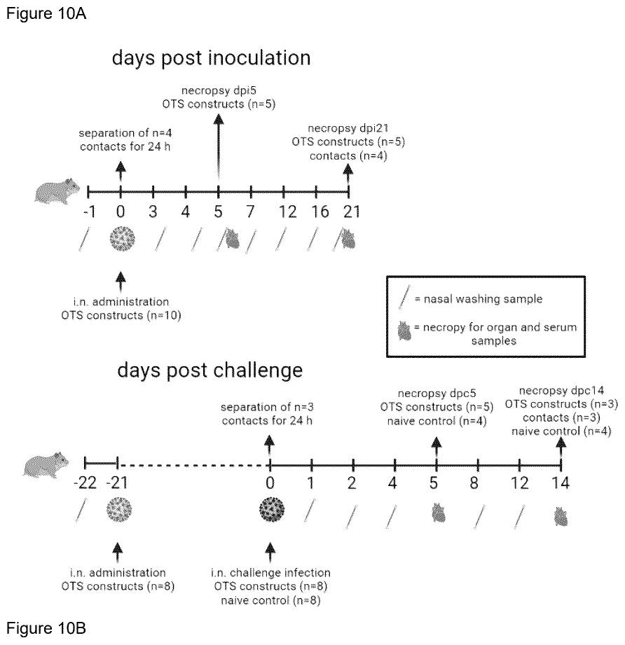

OTS4-5 and OTS7-8 were analyzed for attenuation and protection (FIGS. 6A-6M, FIGS. 7A-7C, FIGS. 8A-8J).

TABLE 1

| | | | OTS | | OTS | | OTS | | |
|---|---|---|---|---|---|---|---|---|---|
| | | SARS- | Fragment 2 | | Fragment 2/3 | | Fragment 4/5 | | OTS |
| Genome | | CoV- | | Nucleo- | | Nucleo- | | Nucleo- | Fragment 7 |
| Anno- | | 2 WT | OTS | tide | OTS | tide | OTS | tide | OTS |
| tation | Genome | WT | codons | changes | codons | changes | codons | changes | codons |
| peptide | Changes | codons | 77 | 136 | 164 | 291 | 191 | 335 | 80 |
| NSP1 | Leu46Leu | CTT | CTT | 0 | TTA | 2 | CTT | 0 | CTT |
| NSP1 | Leu64Leu | CTT | CTT | 0 | TTA | 2 | CTT | 0 | CTT |
| NSP1 | Leu88Leu | CTG | TTG | 1 | TTG | 1 | CTG | 0 | CTG |
| NSP1 | Leu92Leu | CTC | TTG | 2 | TTG | 2 | CTC | 0 | CTC |
| NSP1 | Ser100Ser | AGT | TCA | 3 | TCA | 3 | AGT | 0 | AGT |
| NSP1 | Leu104Leu | CTT | TTA | 2 | TTA | 2 | CTT | 0 | CTT |
| NSP1 | Leu107Leu | CTT | TTA | 2 | TTA | 2 | CTT | 0 | CTT |
| NSP1 | Leu122Leu | CTT | TTA | 2 | TTA | 2 | TTC | 0 | TTC |
| NSP1 | Leu123Leu | CTT | TTA | 2 | TTA | 2 | TTC | 0 | TTC |
| NSP1 | Leu140Leu | CTA | TTA | 1 | TTA | 1 | CTA | 0 | CTA |
| NSP1 | Leu149Leu | CTT | TTA | 2 | TTA | 2 | CTT | 0 | CTT |
| NSP1 | Ser166Ser | AGC | TCG | 3 | TCG | 3 | AGC | 0 | AGC |
| NSP1 | Ser167Ser | AGT | TCA | 3 | TCA | 3 | AGT | 0 | AGT |
| NSP1 | Leu173Leu | CTC | TTG | 2 | TTG | 2 | CTC | 0 | CTC |
| NSP1 | Leu177Leu | CTT | TTA | 2 | TTA | 2 | CTT | 0 | CTT |
| NSP2 | Leu198Leu | CTT | TTA | 2 | TTA | 2 | CTT | 0 | CTT |
| NSP2 | Leu204Leu | CTT | TTA | 2 | TTA | 2 | CTT | 0 | CTT |
| NSP2 | Leu205Leu | CTA | TTA | 1 | TTA | 1 | CTA | 0 | CTA |
| NSP2 | Leu219Leu | CTG | TTG | 1 | TTG | 1 | CTG | 0 | CTG |
| NSP2 | Ser245Ser | TCT | TCA | 1 | TCA | 1 | TCT | 0 | TCT |
| NSP2 | Ser248Ser | AGC | TCG | 3 | TCG | 3 | AGC | 0 | AGC |
| NSP2 | Ser279Ser | TCC | TCG | 1 | TCG | 1 | TCC | 0 | TCC |
| NSP2 | Leu293Leu | CTT | TTA | 2 | TTA | 2 | CTT | 0 | CTT |
| NSP2 | Ser302Ser | TCT | TCA | 1 | TCA | 1 | TCT | 0 | TCT |
| NSP2 | Leu320Leu | CTC | TTG | 2 | TTG | 2 | CTC | 0 | CTC |
| NSP2 | Ser383Ser | AGT | TCA | 3 | TCA | 3 | AGT | 0 | AGT |
| NSP2 | Ser391Ser | TCT | TCA | 1 | TCA | 1 | TCT | 0 | TCT |
| NSP2 | Leu397Leu | CTT | TTA | 2 | TTA | 2 | CTT | 0 | CTT |
| NSP2 | Ser412Ser | TCT | TCA | 1 | TCA | 1 | TCT | 0 | TCT |
| NSP2 | Ser428Ser | AGC | TCG | 3 | TCG | 3 | AGC | 0 | AGC |
| NSP2 | Ser443Ser | TCC | TCG | 1 | TCG | 1 | TCC | 0 | TCC |
| NSP2 | Leu446Leu | CTT | TTA | 2 | TTA | 2 | CTT | 0 | CTT |
| NSP2 | Leu450Leu | CTT | TTA | 2 | TTA | 2 | CTT | 0 | CTT |
| NSP2 | Leu451Leu | CTT | TTA | 2 | TTA | 2 | CTT | 0 | CTT |
| NSP2 | Leu454Leu | CTC | TTG | 2 | TTG | 2 | CTC | 0 | CTC |
| NSP2 | Leu469Leu | CTT | TTA | 2 | TTA | 2 | CTT | 0 | CTT |
| NSP2 | Ser479Ser | TCT | TCA | 1 | TCA | 1 | TCT | 0 | TCT |
| NSP2 | Ser481Ser | TCT | TCA | 1 | TCA | 1 | TCT | 0 | TCT |
| NSP2 | Ser483Ser | TCC | TCG | 1 | TCG | 1 | TCC | 0 | TCC |
| NSP2 | Ser485Ser | AGT | TCA | 3 | TCA | 3 | AGT | 0 | AGT |
| NSP2 | Ser505Ser | TCC | TCG | 1 | TCG | 1 | TCC | 0 | TCC |
| NSP2 | Leu530Leu | CTG | TTG | 1 | TTG | 1 | CTG | 0 | CTG |
| NSP2 | Ser531Ser | AGT | TCA | 3 | TCA | 3 | AGT | 0 | AGT |
| NSP2 | Leu533Leu | CTT | TTG | 2 | TTG | 2 | CTT | 0 | CTT |
| NSP2 | Ser549Ser | TCC | TCG | 1 | TCG | 1 | TCC | 0 | TCC |
| NSP2 | Leu552Leu | CTT | TTA | 2 | TTA | 2 | CTT | 0 | CTT |
| NSP2 | Ser558Ser | TCT | TCA | 1 | TCA | 1 | TCT | 0 | TCT |
| NSP2 | Leu570Leu | CTA | TTA | 1 | TTA | 1 | CTA | 0 | CTA |
| NSP2 | Leu578Leu | CTG | TTG | 1 | TTG | 1 | CTG | 0 | CTG |
| NSP2 | Leu580Leu | CTC | TTG | 2 | TTG | 2 | CTC | 0 | CTC |
| NSP2 | Ser588Ser | TCT | TCA | 1 | TCA | 1 | TCT | 0 | TCT |
| NSP2 | Leu595Leu | CTA | TTA | 1 | TTA | 1 | CTA | 0 | CTA |
| NSP2 | Leu613Leu | CTA | TTA | 1 | TTA | 1 | CTA | 0 | CTA |
| NSP2 | Leu624Leu | CTC | TTG | 2 | TTG | 2 | CTC | 0 | CTC |
| NSP2 | Leu628Leu | CTT | TTA | 2 | TTA | 2 | CTT | 0 | CTT |
| NSP2 | Leu631Leu | CTT | TTA | 2 | TTA | 2 | CTT | 0 | CTT |
| NSP2 | Leu642Leu | CTT | TTA | 2 | TTA | 2 | CTT | 0 | CTT |
| NSP2 | Ser674Ser | AGT | TCA | 3 | TCA | 3 | AGT | 0 | AGT |
| NSP2 | Leu681Leu | CTT | TTA | 2 | TTA | 2 | CTT | 0 | CTT |
| NSP2 | Ser692Ser | TCT | TCA | 1 | TCA | 1 | TCT | 0 | TCT |
| NSP2 | Leu700Leu | CTT | TTA | 2 | TTA | 2 | CTT | 0 | CTT |
| NSP2 | Ser723Ser | TCC | TCG | 1 | TCG | 1 | TCC | 0 | TCC |
| NSP2 | Leu729Leu | CTA | TTA | 1 | TTA | 1 | CTA | 0 | CTA |
| NSP2 | Leu730Leu | CTC | TTG | 2 | TTG | 2 | CTC | 0 | CTC |
| NSP2 | Leu733Leu | CTA | TTA | 1 | TTA | 1 | CTA | 0 | CTA |
| NSP2 | Leu747Leu | CTT | TTA | 2 | TTA | 2 | CTT | 0 | CTT |
| NSP2 | Ser771Ser | AGT | TCA | 3 | TCA | 3 | AGT | 0 | AGT |
| NSP2 | Leu788Leu | CTT | TTA | 2 | TTA | 2 | CTT | 0 | CTT |
| NSP2 | Leu791Leu | CTC | TTG | 2 | TTG | 2 | CTC | 0 | CTC |
| NSP2 | Leu815Leu | CTC | TTG | 2 | TTG | 2 | CTC | 0 | CTC |
| NSP3 | Ser838Ser | AGT | TCA | 3 | TCA | 3 | AGT | 0 | AGT |
| NSP3 | Leu845Leu | CTT | TTA | 2 | TTA | 2 | CTT | 0 | CTT |
| NSP3 | Leu853Leu | CTT | TTA | 2 | TTA | 2 | CTT | 0 | CTT |
| NSP3 | Ser858Ser | TCT | TCA | 1 | TCA | 1 | TCT | 0 | TCT |
| NSP3 | Leu864Leu | CTC | TTG | 2 | TTG | 2 | CTC | 0 | CTC |
| NSP3 | Ser887Ser | TCT | TCA | 1 | TCA | 1 | TCT | 0 | TCT |
| NSP3 | Leu893Leu | CTG | TTG | 1 | TTG | 1 | CTG | 0 | CTG |
| NSP3 | Ser901Ser | AGT | TCA | 3 | TCA | 3 | AGT | 0 | AGT |
| NSP3 | Ser911Ser | TCT | TCA | 1 | TCA | 1 | TCT | 0 | TCT |
| NSP3 | Ser923Ser | TCT | TCT | 0 | TCA | 1 | TCT | 0 | TCT |
| NSP3 | Ser966Ser | TCT | TCT | 0 | TCA | 1 | TCT | 0 | TCT |
| NSP3 | Leu969Leu | CTT | CTT | 0 | TTA | 2 | CTT | 0 | CTT |
| NSP3 | Ser984Ser | AGT | AGT | 0 | TCA | 3 | AGT | 0 | AGT |
| NSP3 | Ser994Ser | AGT | AGT | 0 | TCA | 3 | AGT | 0 | AGT |
| NSP3 | Leu1016Leu | CTT | CTT | 0 | TTA | 2 | CTT | 0 | CTT |
| NSP3 | Leu1034Leu | CTT | CTT | 0 | TTA | 2 | CTT | 0 | CTT |
| NSP3 | Leu1065Leu | CTT | CTT | 0 | TTA | 2 | CTT | 0 | CTT |
| NSP3 | Ser1087Ser | TCT | TCT | 0 | TCA | 1 | TCT | 0 | TCT |
| NSP3 | Leu1097Leu | CTT | CTT | 0 | TTA | 2 | CTT | 0 | CTT |
| NSP3 | Ser1102Ser | AGT | AGT | 0 | TCA | 3 | AGT | 0 | AGT |
| NSP3 | Leu1110Leu | CTT | CTT | 0 | TTA | 2 | CTT | 0 | CTT |
| NSP3 | Leu1115Leu | CTT | CTT | 0 | TTA | 2 | CTT | 0 | CTT |
| NSP3 | Leu1130Leu | CTT | CTT | 0 | TTA | 2 | CTT | 0 | CTT |
| NSP3 | Leu1131Leu | CTT | CTT | 0 | TTA | 2 | CTT | 0 | CTT |
| NSP3 | Ser1133Ser | AGT | AGT | 0 | TCA | 3 | AGT | 0 | AGT |
| NSP3 | Leu1144Leu | CTA | CTA | 0 | TTA | 1 | CTA | 0 | CTA |
| NSP3 | Leu1145Leu | CTT | CTT | 0 | TTA | 2 | CTT | 0 | CTT |
| NSP3 | Ser1161Ser | TCT | TCT | 0 | TCA | 1 | TCT | 0 | TCT |
| NSP3 | Leu1182Leu | CTC | CTC | 0 | TTG | 2 | CTC | 0 | CTC |
| NSP3 | Leu1186Leu | CTT | CTT | 0 | TTA | 2 | CTT | 0 | CTT |
| NSP3 | Ser1189Ser | AGC | AGC | 0 | TCG | 3 | AGC | 0 | AGC |
| NSP3 | Ser1195Ser | AGT | AGT | 0 | TCA | 3 | AGT | 0 | AGT |
| NSP3 | Leu1243Leu | CTG | CTG | 0 | TTG | 1 | CTG | 0 | CTG |
| NSP3 | Leu1249Leu | CTC | CTC | 0 | TTG | 2 | CTC | 0 | CTC |
| NSP3 | Leu1255Leu | CTT | CTT | 0 | TTA | 2 | CTT | 0 | CTT |
| NSP3 | Leu1263Leu | CTT | CTT | 0 | TTA | 2 | CTT | 0 | CTT |
| NSP3 | Leu1270Leu | CTT | CTT | 0 | TTA | 2 | CTT | 0 | CTT |
| NSP3 | Ser1272Ser | AGT | AGT | 0 | TCA | 3 | AGT | 0 | AGT |
| NSP3 | Leu1313Leu | CTA | CTA | 0 | TTA | 1 | CTA | 0 | CTA |
| NSP3 | Leu1346Leu | CTT | CTT | 0 | TTA | 2 | CTT | 0 | CTT |
| NSP3 | Leu1356Leu | CTA | CTA | 0 | TTA | 1 | CTA | 0 | CTA |
| NSP3 | Ser1358Ser | TCT | TCT | 0 | TCA | 1 | TCT | 0 | TCT |
| NSP3 | Ser1361Ser | TCT | TCT | 0 | TCA | 1 | TCT | 0 | TCT |
| NSP3 | Leu1268Leu | CTT | CTT | 0 | TTA | 2 | CTT | 0 | CTT |
| NSP3 | Ser1372Ser | TCT | TCT | 0 | TCA | 1 | TCT | 0 | TCT |
| NSP3 | Leu1379Leu | CTT | CTT | 0 | TTA | 2 | CTT | 0 | CTT |
| NSP3 | Ser1427Ser | AGT | AGT | 0 | TCA | 3 | AGT | 0 | AGT |
| NSP3 | Leu1434Leu | CTT | CTT | 0 | TTA | 2 | CTT | 0 | CTT |
| NSP3 | Leu1438Leu | CTT | CTT | 0 | TTA | 2 | CTT | 0 | CTT |
| NSP3 | Leu1441Leu | CTA | CTA | 0 | TTA | 1 | CTA | 0 | CTA |
| NSP3 | Leu1445Leu | CTT | CTT | 0 | TTA | 2 | CTT | 0 | CTT |
| NSP3 | Leu1450Leu | CTT | CTT | 0 | TTA | 2 | CTT | 0 | CTT |
| NSP3 | Leu1469Leu | CTC | CTC | 0 | TTG | 2 | CTC | 0 | CTC |
| NSP3 | Ser1476Ser | TCT | TCT | 0 | TCA | 1 | TCT | 0 | TCT |
| NSP3 | Ser1478Ser | TCT | TCT | 0 | TCA | 1 | TCT | 0 | TCT |
| NSP3 | Leu1490Leu | CTT | CTT | 0 | TTA | 2 | CTT | 0 | CTT |
| NSP3 | Ser1492Ser | TCT | TCT | 0 | TCA | 1 | TCT | 0 | TCT |
| NSP3 | Ser1493Ser | TCT | TCT | 0 | TCA | 1 | TCT | 0 | TCT |
| NSP3 | Ser1494Ser | TCT | TCT | 0 | TCA | 1 | TCT | 0 | TCT |
| NSP3 | Leu1507Leu | CTT | CTT | 0 | TTA | 2 | CTT | 0 | CTT |
| NSP3 | Ser1510Ser | TCC | TCC | 0 | TCG | 1 | TCC | 0 | TCC |
| NSP3 | Ser1515Ser | TCC | TCC | 0 | TCG | 1 | TCC | 0 | TCC |
| NSP3 | Ser1517Ser | TCT | TCT | 0 | TCA | 1 | TCT | 0 | TCT |
| NSP3 | Ser1520Ser | TCT | TCT | 0 | TCA | 1 | TCT | 0 | TCT |
| NSP3 | Leu1528Leu | CTT | CTT | 0 | TTA | 2 | CTT | 0 | CTT |
| NSP3 | Ser1534Ser | AGT | AGT | 0 | TCA | 3 | AGT | 0 | AGT |
| NSP3 | Ser1539Ser | AGT | AGT | 0 | TCA | 3 | AGT | 0 | AGT |
| NSP3 | Leu1546Leu | CTA | CTA | 0 | TTA | 1 | CTA | 0 | CTA |
| NSP3 | Leu1556Leu | CTT | CTT | 0 | TTA | 2 | CTT | 0 | CTT |
| NSP3 | Leu1559Leu | CTT | CTT | 0 | TTA | 2 | CTT | 0 | CTT |
| NSP3 | Leu1560Leu | CTT | CTT | 0 | TTA | 2 | CTT | 0 | CTT |
| NSP3 | Ser1561Ser | TCT | TCT | 0 | TCA | 1 | TCT | 0 | TCT |
| NSP3 | Leu1579Leu | TTG | TTG | 0 | CTC | 2 | TTG | 0 | TTG |
| NSP3 | Leu1627Leu | CTA | CTA | 0 | TTA | 1 | CTA | 0 | CTA |
| NSP3 | Ser1641Ser | AGT | AGT | 0 | TCA | 3 | AGT | 0 | AGT |
| NSP3 | Leu1643Leu | CTG | CTG | 0 | TTG | 1 | CTG | 0 | CTG |
| NSP3 | Ser1666Ser | TCT | TCT | 0 | TCA | 1 | TCT | 0 | TCT |
| NSP3 | Leu1676Leu | CTT | CTT | 0 | TTA | 2 | CTT | 0 | CTT |
| NSP3 | Leu1683Leu | CTC | CTC | 0 | TTG | 2 | CTC | 0 | CTC |
| NSP3 | Leu1695Leu | CTA | CTA | 0 | TTA | 1 | CTA | 0 | CTA |
| NSP3 | Leu1713Leu | CTT | CTT | 0 | TTA | 2 | CTT | 0 | CTT |
| NSP3 | Ser1733Ser | AGT | AGT | 0 | TCA | 3 | AGT | 0 | AGT |
| NSP3 | Ser1743Ser | TCT | TCT | 0 | TCA | 1 | TCT | 0 | TCT |
| NSP3 | Leu1762Leu | CTT | CTT | 0 | TTA | 2 | CTT | 0 | CTT |
| NSP3 | Leu1774Leu | CTT | CTT | 0 | TTA | 2 | CTT | 0 | CTT |
| NSP3 | Ser1775Ser | TCT | TCT | 0 | TCA | 1 | TCT | 0 | TCT |
| NSP3 | Leu1797Leu | CTA | CTA | 0 | TTA | 1 | CTA | 0 | CTA |
| NSP3 | Leu1816Leu | CTT | CTT | 0 | TTA | 2 | CTT | 0 | CTT |
| NSP3 | Ser1825Ser | AGT | AGT | 0 | TCA | 3 | AGT | 0 | AGT |
| NSP3 | Ser1841Ser | TCT | TCT | 0 | TCA | 1 | TCT | 0 | TCT |
| NSP3 | Leu1853Leu | CTT | CTT | 0 | TTA | 2 | CTT | 0 | CTT |
| NSP3 | Ser2856Ser | TCC | TCC | 0 | TCG | 1 | TCC | 0 | TCC |
| NSP3 | Ser1872Ser | AGT | AGT | 0 | TCA | 3 | AGT | 0 | AGT |
| NSP3 | Ser1905Ser | TCT | TCT | 0 | TCA | 1 | TCT | 0 | TCT |
| NSP3 | Leu2028Leu | CTG | CTG | 0 | CTG | 0 | TTG | 1 | CTG |
| NSP3 | Leu2039Leu | CTT | CTT | 0 | CTT | 0 | TTA | 2 | CTT |
| NSP3 | Leu2044Leu | CTA | CTA | 0 | CTA | 0 | TTA | 1 | CTA |
| NSP3 | Ser2048Ser | TCT | TCT | 0 | TCT | 0 | TCA | 1 | TCT |
| NSP3 | Leu2062Leu | CTT | CTT | 0 | CTT | 0 | TTA | 2 | CTT |
| NSP3 | Leu2077Leu | CTT | CTT | 0 | CTT | 0 | TTA | 2 | CTT |
| NSP3 | Ser2083Ser | AGT | AGT | 0 | AGT | 0 | TCA | 3 | AGT |
| NSP3 | Leu2095Leu | TTA | TTA | 0 | TTA | 0 | CTA | 1 | TTA |
| NSP3 | Ser2103Ser | TCT | TCT | 0 | TCT | 0 | TCA | 1 | TCT |
| NSP3 | Ser2104Ser | AGT | AGT | 0 | AGT | 0 | TCA | 3 | AGT |
| NSP3 | Ser2114Ser | TCT | TCT | 0 | TCT | 0 | TCA | 1 | TCT |
| NSP3 | Leu2122Leu | CTT | CTT | 0 | CTT | 0 | TTA | 2 | CTT |
| NSP3 | Ser2132Ser | AGT | AGT | 0 | AGT | 0 | TCA | 3 | AGT |
| NSP3 | Leu2146Leu | CTT | CTT | 0 | CTT | 0 | TTA | 2 | CTT |
| NSP3 | Ser2151Ser | AGT | AGT | 0 | AGT | 0 | TCA | 3 | AGT |
| NSP3 | Leu2177Leu | CTA | CTA | 0 | CTA | 0 | TTA | 1 | CTA |
| NSP3 | Ser2185Ser | AGT | AGT | 0 | AGT | 0 | TCA | 3 | AGT |
| NSP3 | Ser2188Ser | TCT | TCT | 0 | TCT | 0 | TCA | 1 | TCT |
| NSP3 | Ser2193Ser | TCT | TCT | 0 | TCT | 0 | TCA | 1 | TCT |
| NSP3 | Ser2205Ser | AGT | AGT | 0 | AGT | 0 | TCA | 3 | AGT |
| NSP3 | Leu2211Leu | CTA | CTA | 0 | CTA | 0 | TTA | 1 | CTA |
| NSP3 | Ser2224Ser | TCT | TCT | 0 | TCT | 0 | TCA | 1 | TCT |
| NSP3 | Leu2226Leu | CTG | CTG | 0 | CTG | 0 | TTG | 1 | CTG |
| NSP3 | Leu2235Leu | CTA | CTA | 0 | CTA | 0 | TTA | 1 | CTA |
| NSP3 | Ser2237Ser | AGT | AGT | 0 | AGT | 0 | TCA | 3 | AGT |
| NSP3 | Leu2240Leu | CTA | CTA | 0 | CTA | 0 | TTA | 1 | CTA |
| NSP3 | Ser2242Ser | TCT | TCT | 0 | TCT | 0 | TCA | 1 | TCT |
| NSP3 | Ser2255Ser | TCT | TCT | 0 | TCT | 0 | TCA | 1 | TCT |
| NSP3 | Ser2261Ser | TCT | TCT | 0 | TCT | 0 | TCA | 1 | TCT |
| NSP3 | Ser2273Ser | TCT | TCT | 0 | TCT | 0 | TCA | 1 | TCT |
| NSP3 | Ser2285Ser | TCT | TCT | 0 | TCT | 0 | TCA | 1 | TCT |
| NSP3 | Ser2289Ser | AGT | AGT | 0 | AGT | 0 | TCA | 3 | AGT |
| NSP3 | Leu2292Leu | CTT | CTT | 0 | CTT | 0 | TTA | 2 | CTT |
| NSP3 | Ser2293Ser | AGT | AGT | 0 | AGT | 0 | TCA | 3 | AGT |
| NSP3 | Ser2297Ser | TCT | TCT | 0 | TCT | 0 | TCA | 1 | TCT |
| NSP3 | Ser3303Ser | TCT | TCT | 0 | TCT | 0 | TCA | 1 | TCT |
| NSP3 | Ser2313Ser | TCT | TCT | 0 | TCT | 0 | TCA | 1 | TCT |
| NSP3 | Leu2333Leu | CTT | CTT | 0 | CTT | 0 | TTA | 2 | CTT |
| NSP3 | Leu2341Leu | CTT | CTT | 0 | CTT | 0 | TTA | 2 | CTT |
| NSP3 | Ser2352Ser | AGC | AGC | 0 | AGC | 0 | TCG | 3 | AGC |
| NSP3 | Ser2360Ser | AGT | AGT | 0 | AGT | 0 | TCA | 3 | AGT |
| NSP3 | Ser2362Ser | TCT | TCT | 0 | TCT | 0 | TCA | 1 | TCT |
| NSP3 | Leu2364Leu | CTT | CTT | 0 | CTT | 0 | TTA | 2 | CTT |
| NSP3 | Leu2371Leu | CTT | CTT | 0 | CTT | 0 | TTA | 2 | CTT |
| NSP3 | Ser2396Ser | AGT | AGT | 0 | AGT | 0 | TCA | 3 | AGT |
| NSP3 | Ser2433Ser | TCC | TCC | 0 | TCC | 0 | TCG | 1 | TCC |
| NSP3 | Leu2447Leu | CTA | CTA | 0 | CTA | 0 | TTA | 1 | CTA |
| NSP3 | Ser2462Ser | AGT | AGT | 0 | AGT | 0 | TCA | 3 | AGT |
| NSP3 | Ser2466Ser | AGT | AGT | 0 | AGT | 0 | TCA | 3 | AGT |
| NSP3 | Leu2475Leu | CTA | CTA | 0 | CTA | 0 | TTA | 1 | CTA |
| NSP3 | Ser2487Ser | TCT | TCT | 0 | TCT | 0 | TCA | 1 | TCT |
| NSP3 | Ser2488Ser | TCT | TCT | 0 | TCT | 0 | TCA | 1 | TCT |
| NSP3 | Ser2493Ser | AGT | AGT | 0 | AGT | 0 | TCA | 3 | AGT |
| NSP3 | Ser2500Ser | TCC | TCC | 0 | TCC | 0 | TCG | 1 | TCC |
| NSP3 | Leu2503Leu | CTT | CTT | 0 | CTT | 0 | TTA | 2 | CTT |
| NSP3 | Ser2517Ser | TCT | TCT | 0 | TCT | 0 | TCA | 1 | TCT |
| NSP3 | Leu2518Leu | CTC | CTC | 0 | CTC | 0 | TTG | 2 | CTC |
| NSP3 | Ser2519Ser | TCT | TCT | 0 | TCT | 0 | TCA | 1 | TCT |
| NSP3 | Leu2527Leu | CTG | CTG | 0 | CTG | 0 | TTG | 1 | CTG |
| NSP3 | Ser2553Ser | TCT | TCT | 0 | TCT | 0 | TCA | 1 | TCT |
| NSP3 | Ser2558Ser | TCT | TCT | 0 | TCT | 0 | TCA | 1 | TCT |
| NSP3 | Leu2564Leu | CTT | CTT | 0 | CTT | 0 | TTA | 2 | CTT |
| NSP3 | Leu2570Leu | CTG | CTG | 0 | CTG | 0 | TTG | 1 | CTG |
| NSP3 | Leu2572Leu | CTA | CTA | 0 | CTA | 0 | TTA | 1 | CTA |
| NSP3 | Ser2578Ser | TCT | TCT | 0 | TCT | 0 | TCA | 1 | TCT |
| NSP3 | Ser2583Ser | AGT | AGT | 0 | AGT | 0 | TCA | 3 | AGT |
| NSP3 | Leu2609Leu | CTC | CTC | 0 | CTC | 0 | TTG | 2 | CTC |
| NSP3 | Leu2612Leu | CTA | CTA | 0 | CTA | 0 | TTA | 1 | CTA |
| NSP3 | Leu2620Leu | CTT | CTT | 0 | CTT | 0 | TTA | 2 | CTT |
| NSP3 | Ser2625Ser | TCC | TCC | 0 | TCC | 0 | TCG | 1 | TCC |
| NSP3 | Ser2631Ser | TCT | TCT | 0 | TCT | 0 | TCA | 1 | TCT |
| NSP3 | Leu2655Leu | CTT | CTT | 0 | CTT | 0 | TTA | 2 | CTT |
| NSP3 | Ser2661Ser | TCT | TCT | 0 | TCT | 0 | TCA | 1 | TCT |
| NSP3 | Ser2669Ser | AGT | AGT | 0 | AGT | 0 | TCA | 3 | AGT |
| NSP3 | Leu2675Leu | CTC | CTC | 0 | CTC | 0 | TTG | 2 | CTC |
| NSP3 | Leu2688Leu | CTT | CTT | 0 | CTT | 0 | TTA | 2 | CTT |
| NSP3 | Ser2695Ser | AGT | AGT | 0 | AGT | 0 | TCA | 3 | AGT |
| NSP3 | Ser2706Ser | AGT | AGT | 0 | AGT | 0 | TCA | 3 | AGT |
| NSP3 | Ser2722Ser | TCT | TCT | 0 | TCT | 0 | TCA | 1 | TCT |
| NSP3 | Leu2725Leu | CTA | CTA | 0 | CTA | 0 | TTA | 1 | CTA |
| NSP3 | Ser2731Ser | AGT | AGT | 0 | AGT | 0 | TCA | 3 | AGT |
| NSP3 | Leu2760Leu | CTT | CTT | 0 | CTT | 0 | TTA | 2 | CTT |
| NSP4 | Leu2778Leu | CTT | CTT | 0 | CTT | 0 | TTA | 2 | CTT |
| NSP4 | Leu2781Leu | CTT | CTT | 0 | CTT | 0 | TTA | 2 | CTT |
| NSP4 | Ser2797Ser | TCT | TCT | 0 | TCT | 0 | TCA | 1 | TCT |
| NSP4 | Ser2804Ser | AGT | AGT | 0 | AGT | 0 | TCA | 3 | AGT |
| NSP4 | Ser2822Ser | TCT | TCT | 0 | TCT | 0 | TCA | 1 | TCT |
| NSP4 | Ser2839Ser | AGC | AGC | 0 | AGC | 0 | TCG | 3 | AGC |
| NSP4 | Ser2844Ser | AGT | AGT | 0 | AGT | 0 | TCA | 3 | AGT |
| NSP4 | Ser2890Ser | AGT | AGT | 0 | AGT | 0 | TCA | 3 | AGT |
| NSP4 | Leu2902Leu | CTT | CTT | 0 | CTT | 0 | TTA | 2 | CTT |
| NSP4 | Ser2926Ser | TCT | TCT | 0 | TCT | 0 | TCA | 1 | TCT |
| NSP4 | Leu2939Leu | CTA | CTA | 0 | CTA | 0 | TTA | 1 | CTA |
| NSP4 | Ser2942Ser | TCT | TCT | 0 | TCT | 0 | TCA | 1 | TCT |
| NSP4 | Ser2947Ser | AGT | AGT | 0 | AGT | 0 | TCA | 3 | AGT |
| NSP4 | Leu2956Leu | CTC | CTC | 0 | CTC | 0 | TTG | 2 | CTC |
| NSP4 | Ser2960Ser | TCT | TCT | 0 | TCT | 0 | TCA | 1 | TCT |
| NSP4 | Leu2969Leu | CTT | CTT | 0 | CTT | 0 | TTA | 2 | CTT |
| NSP4 | Ser2972Ser | TCT | TCT | 0 | TCT | 0 | TCA | 1 | TCT |
| NSP4 | Ser2981Ser | TCT | TCT | 0 | TCT | 0 | TCA | 1 | TCT |
| NSP4 | Ser2999Ser | TCT | TCT | 0 | TCT | 0 | TCA | 1 | TCT |
| NSP4 | Ser3001Ser | AGT | AGT | 0 | AGT | 0 | TCA | 3 | AGT |
| NSP4 | Leu3006Leu | CTT | CTT | 0 | CTT | 0 | TTA | 2 | CTT |
| NSP4 | Ser3013Ser | TCT | TCT | 0 | TCT | 0 | TCA | 1 | TCT |
| NSP4 | Leu3027Leu | CTT | CTT | 0 | CTT | 0 | TTA | 2 | CTT |
| NSP4 | Leu3034Leu | CTA | CTA | 0 | CTA | 0 | TTA | 1 | CTA |
| NSP4 | Ser3046Ser | TCT | TCT | 0 | TCT | 0 | TCA | 1 | TCT |
| NSP4 | Leu3060Leu | CTT | CTT | 0 | CTT | 0 | TTA | 2 | CTT |
| NSP4 | Ser3075Ser | AGT | AGT | 0 | AGT | 0 | TCA | 3 | AGT |
| NSP4 | Leu3084Leu | CTA | CTA | 0 | CTA | 0 | TTA | 1 | CTA |
| NSP4 | Leu3086Leu | CTT | CTT | 0 | CTT | 0 | TTA | 2 | CTT |
| NSP4 | Leu3092Leu | CTC | CTC | 0 | CTC | 0 | TGG | 2 | CTC |
| NSP4 | Ser3106Ser | TCT | TCT | 0 | TCT | 0 | TCA | 1 | TCT |
| NSP4 | Leu3116Leu | CTT | CTT | 0 | CTT | 0 | TTA | 2 | CTT |
| NSP4 | Ser3121Ser | TCT | TCT | 0 | TCT | 0 | TCA | 1 | TCT |
| NSP4 | Ser3149Ser | TCC | TCC | 0 | TCC | 0 | TCG | 1 | TCC |
| NSP4 | Ser3158Ser | AGT | AGT | 0 | AGT | 0 | TCA | 3 | AGT |
| NSP4 | Leu3161Leu | CTA | CTA | 0 | CTA | 0 | TTA | 1 | CTA |
| NSP4 | Ser3171Ser | TCC | TCC | 0 | TCC | 0 | TCG | 1 | TCC |
| NSP4 | Ser3173Ser | AGT | AGT | 0 | AGT | 0 | TCA | 2 | AGT |
| NSP4 | Leu3180Leu | CTG | CTG | 0 | CTG | 0 | TTG | 1 | CTG |
| NSP4 | Leu3191LEu | CTA | CTA | 0 | CTA | 0 | TTA | 1 | CTA |
| NSP4 | Ser3195Ser | AGT | AGT | 0 | AGT | 0 | TCA | 3 | AGT |
| NSP4 | Leu3198Leu | CTA | CTA | 0 | CTA | 0 | TTA | 1 | CTA |
| NSP4 | Leu3201Leu | CTT | CTT | 0 | CTT | 0 | TTA | 2 | CTT |
| NSP4 | Leu3210Leu | CTT | CTT | 0 | CTT | 0 | TTA | 2 | CTT |
| NSP4 | Ser3218Ser | AGT | AGT | 0 | AGT | 0 | TCA | 3 | AGT |
| NSP4 | Ser3225Ser | AGC | AGC | 0 | AGC | 0 | TCG | 3 | AGC |
| NSP4 | Leu3234Leu | CTC | CTC | 0 | CTC | 0 | TTG | 2 | CTC |
| NSP4 | Leu3238Leu | CTC | CTC | 0 | CTC | 0 | TTG | 2 | CTC |
| NSP4 | Ser3242Ser | AGT | AGT | 0 | AGT | 0 | TCA | 3 | AGT |
| NSP4 | Ser3246Ser | TCT | TCT | 0 | TCT | 0 | TCA | 1 | TCT |
| NSP4 | Leu3249Leu | CTT | CTT | 0 | CTT | 0 | TTA | 2 | CTT |
| NSP4 | Ser3256Ser | TCT | TCT | 0 | TCT | 0 | TCA | 1 | TCT |
| NSP5 | Ser3264Ser | AGT | AGT | 0 | AGT | 0 | TCA | 3 | AGT |
| NSP5 | Ser3273Ser | TCT | TCT | 0 | TCT | 0 | TCA | 1 | TCT |
| NSP5 | Leu3290Leu | CTT | CTT | 0 | CTT | 0 | TTA | 2 | CTT |
| NSP5 | Leu3293Leu | CTT | CTT | 0 | CTT | 0 | TTA | 2 | CTT |
| NSP5 | Leu3295Leu | CTT | CTT | 0 | CTT | 0 | TTA | 2 | CTT |
| NSP5 | Ser3309Ser | TCT | TCT | 0 | TCT | 0 | TCA | 1 | TCT |
| NSP5 | Leu3313Leu | CTT | CTT | 0 | CTT | 0 | TTA | 2 | CTT |
| NSP5 | Leu3321Leu | CTC | CTC | 0 | CTC | 0 | TTG | 2 | CTC |
| NSP5 | Ser3325Ser | TCT | TCT | 0 | TCT | 0 | TCA | 1 | TCT |
| NSP5 | Leu3338Leu | CTC | CTC | 0 | CTC | 0 | TTG | 2 | CTC |
| NSP5 | Ser3344Ser | TCT | TCT | 0 | TCT | 0 | TCA | 1 | TCT |
| NSP5 | Leu3350Leu | CTT | CTT | 0 | CTT | 0 | TTA | 2 | CTT |
| NSP5 | Leu3352Leu | CTT | CTT | 0 | CTT | 0 | TTA | 2 | CTT |
| NSP5 | Ser3386Ser | TCT | TCT | 0 | TCT | 0 | TCA | 1 | TCT |
| NSP5 | Leu3404Leu | TTA | TTA | 0 | TTA | 0 | CTT | 2 | TTA |
| NSP5 | Ser3410Ser | AGT | AGT | 0 | AGT | 0 | TCA | 3 | AGT |
| NSP5 | Ser3421Ser | TCT | TCT | 0 | TCT | 0 | TCA | 1 | TCT |
| NSP5 | Leu3483Leu | CTC | CTC | 0 | CTC | 0 | TTG | 2 | CTC |
| NSP5 | Leu3490Leu | CTT | CTT | 0 | CTT | 0 | TTA | 2 | CTT |
| NSP5 | Leu3495Leu | CTT | CTT | 0 | CTT | 0 | TTA | 2 | CTT |
| NSP5 | Leu3505Leu | CTA | CTA | 0 | CTA | 0 | TTA | 1 | CTA |
| NSP5 | Leu3513Leu | CTA | CTA | 0 | CTA | 0 | TTA | 1 | CTA |
| NSP5 | Leu3516Leu | CTT | CTT | 0 | CTT | 0 | TTA | 2 | CTT |
| NSP5 | Leu3535Leu | CTG | CTG | 0 | CTG | 0 | TTG | 1 | CTG |
| NSP5 | Ser3547Ser | AGT | AGT | 0 | AGT | 0 | TCA | 3 | AGT |
| NSP6 | Ser3570Ser | AGT | AGT | 0 | AGT | 0 | TCA | 3 | AGT |
| NSP6 | Leu3585Leu | CTC | CTC | 0 | CTC | 0 | TTG | 2 | CTC |
| NSP6 | Leu3581Leu | CTT | CTT | 0 | CTT | 0 | TTA | 2 | CTT |
| NSP6 | Ser3597Ser | AGT | AGT | 0 | AGT | 0 | TCA | 3 | AGT |
| NSP6 | Ser3601Ser | TCT | TCT | 0 | TCT | 0 | TCA | 1 | TCT |
| NSP6 | Ser3622Ser | TCT | TCT | 0 | TCT | 0 | TCA | 1 | TCT |
| NSP6 | Leu3636Leu | CTC | CTC | 0 | CTC | 0 | TTG | 2 | CTC |
| NSP6 | Ser3643Ser | TCT | TCT | 0 | TCT | 0 | TCA | 1 | TCT |
| NSP6 | Leu3644Leu | CTT | CTT | 0 | CTT | 0 | TTA | 2 | CTT |
| NSP6 | Ser3658Ser | AGT | AGT | 0 | AGT | 0 | TCA | 3 | AGT |
| NSP6 | Ser3673Ser | AGT | AGT | 0 | AGT | 0 | TCA | 3 | AGT |
| NSP6 | Ser3675Ser | TCT | TCT | 0 | TCT | 0 | TCA | 1 | TCT |
| NSP6 | Leu3679Leu | CTA | CTA | 0 | CTA | 0 | TTA | 1 | CTA |
| NSP6 | Leu3692Leu | CTA | CTA | 0 | CTA | 0 | TTA | 1 | CTA |
| NSP6 | Leu3694Leu | CTT | CTT | 0 | CTT | 0 | TTA | 2 | CTT |
| NSP6 | Leu3711Leu | CTT | CTT | 0 | CTT | 0 | TTA | 2 | CTT |
| NSP6 | Leu3717Leu | CTC | CTC | 0 | CTC | 0 | TTG | 2 | CTC |
| NSP6 | Ser3732Ser | TCC | TCC | 0 | TCC | 0 | TCG | 1 | TCC |
| NSP6 | Leu3736Leu | CTT | CTT | 0 | CTT | 0 | TTA | 2 | CTT |
| NSP6 | Ser3739Ser | TCT | TCT | 0 | TCT | 0 | TCA | 1 | TCT |
| NSP6 | Ser3742Ser | TCT | TCT | 0 | TCT | 0 | TCA | 1 | TCT |
| NSP6 | Leu3776Leu | CTT | CTT | 0 | CTT | 0 | TTA | 2 | CTT |
| NSP6 | Leu3781Leu | CTA | CTA | 0 | CTA | 0 | TTA | 2 | CTA |
| NSP6 | Leu3796Leu | CTC | CTC | 0 | CTC | 0 | TTG | 2 | CTC |
| NSP6 | Leu3828Leu | CTA | CTA | 0 | CTA | 0 | TTA | 1 | CTA |
| NSP6 | Leu3829Leu | CTC | CTC | 0 | CTC | 0 | TTG | 2 | CTC |
| NSP6 | Ser3834Ser | AGC | AGC | 0 | AGC | 0 | TCG | 3 | AGC |
| NSP6 | Leu3840Leu | CTC | CTC | 0 | CTC | 0 | TTG | 2 | CTC |
| NSP7 | Ser3860Ser | TCT | TCT | 0 | TCT | 0 | TCA | 1 | TCT |
| NSP7 | Leu3873Leu | CTC | CTC | 0 | CTC | 0 | TTG | 2 | CTC |
| NSP7 | Leu3879Leu | CTC | CTC | 0 | CTC | 0 | TTG | 2 | CTC |
| NSP7 | Ser3885Ser | TCT | TCT | 0 | TCT | 0 | TCA | 1 | TCT |
| NSP12 | Leu4792Leu | CTT | CTT | 0 | CTT | 0 | CTT | 0 | TTA |
| NSP12 | Ser4816Ser | TCT | TCT | 0 | TCT | 0 | TCT | 0 | TCA |
| NSP12 | Ser4824Ser | AGT | AGT | 0 | AGT | 0 | AGT | 0 | TCA |
| NSP12 | Ser4825Ser | TCT | TCT | 0 | TCT | 0 | TCT | 0 | TCA |
| NSP12 | Ser4842Ser | AGC | AGC | 0 | AGC | 0 | AGC | 0 | TCG |
| NSP12 | Leu4851Leu | CTA | CTA | 0 | CTA | 0 | CTA | 0 | TTA |
| NSP12 | Leu4860Leu | CTA | CTA | 0 | CTA | 0 | CTA | 0 | TTA |
| NSP12 | Leu4861Leu | CTA | CTA | 0 | CTA | 0 | CTA | 0 | TTA |
| NSP12 | Leu4889Leu | CTA | CTA | 0 | CTA | 0 | CTA | 0 | TTA |
| NSP12 | Leu4905Leu | CTT | CTT | 0 | CTT | 0 | CTT | 0 | TTA |
| NSP12 | Ser4911Ser | AGT | AGT | 0 | AGT | 0 | AGT | 0 | TCA |
| NSP12 | Leu4918Leu | CTT | CTT | 0 | CTT | 0 | CTT | 0 | TTA |
| NSP12 | Leu4935Leu | CTT | CTT | 0 | CTT | 0 | CTT | 0 | TTA |
| NSP12 | Ser4940Ser | AGT | AGT | 0 | AGT | 0 | AGT | 0 | TCA |
| NSP12 | Ser4952Ser | TCT | TCT | 0 | TCT | 0 | TCT | 0 | TCA |
| NSP12 | Ser4955Ser | AGT | AGT | 0 | AGT | 0 | AGT | 0 | TCA |
| NSP12 | Ser4983Ser | AGC | AGC | 0 | AGC | 0 | AGC | 0 | TCG |
| NSP12 | Ser4998Ser | AGT | AGT | 0 | AGT | 0 | AGT | 0 | TCA |
| NSP12 | Leu5005Leu | CTT | CTT | 0 | CTT | 0 | CTT | 0 | TTA |
| NSP12 | Leu5021Leu | CTT | CTT | 0 | CTT | 0 | CTT | 0 | TTA |
| NSP12 | Leu5027Leu | CTT | CTT | 0 | CTT | 0 | CTT | 0 | TTA |
| NSP12 | Leu5029Leu | CTT | CTT | 0 | CTT | 0 | CTT | 0 | TTA |
| NSP12 | Ser5038Ser | AGC | AGC | 0 | AGC | 0 | AGC | 0 | TCG |
| NSP12 | Ser5055Ser | AGT | AGT | 0 | AGT | 0 | AGT | 0 | TCA |
| NSP12 | Leu5064Leu | CTA | CTA | 0 | CTA | 0 | CTA | 0 | TTA |
| NSP12 | Ser5083Ser | AGT | AGT | 0 | AGT | 0 | AGT | 0 | TCA |
| NSP12 | Leu5098Leu | CTT | CTT | 0 | CTT | 0 | CTT | 0 | TTA |
| NSP12 | Ser5100Ser | TCT | TCT | 0 | TCT | 0 | TCT | 0 | TCA |
| NSP12 | Leu5118Leu | CTT | CTT | 0 | CTT | 0 | CTT | 0 | TTA |
| NSP12 | Leu5122Leu | CTC | CTC | 0 | CTC | 0 | CTC | 0 | TTG |
| NSP12 | Leu5149Leu | CTC | CTC | 0 | CTC | 0 | CTC | 0 | TTG |
| NSP12 | Ser5150Ser | TCT | TCT | 0 | TCT | 0 | TCT | 0 | TCA |
| NSP12 | Ser5159Ser | AGC | AGC | 0 | AGC | 0 | AGC | 0 | TCG |
| NSP12 | Ser5163Ser | TCT | TCT | 0 | TCT | 0 | TCT | 0 | TCA |
| NSP12 | Leu5166Leu | CTA | CTA | 0 | CTA | 0 | CTA | 0 | TTA |
| NSP12 | Ser5169Ser | AGC | AGC | 0 | AGC | 0 | AGC | 0 | TCG |
| NSP12 | Leu5177Leu | CTT | CTT | 0 | CTT | 0 | CTT | 0 | TTA |
| NSP12 | Ser5186Ser | TCT | TCT | 0 | TCT | 0 | TCT | 0 | TCA |
| NSP12 | Leu5196Leu | CTT | CTT | 0 | CTT | 0 | CTT | 0 | TTA |
| NSP12 | Ser5205Ser | TCT | TCT | 0 | TCT | 0 | TCT | 0 | TCA |
| NSP12 | Leu5210Leu | CTA | CTA | 0 | CTA | 0 | CTA | 0 | TTA |
| NSP12 | Leu5220Leu | CTT | CTT | 0 | CTT | 0 | CTT | 0 | TTA |
| NSP12 | Leu5229Leu | CTA | CTA | 0 | CTA | 0 | CTA | 0 | TTA |
| NSP12 | Leu5245Leu | CTT | CTT | 0 | CTT | 0 | CTT | 0 | TTA |
| NSP12 | Ser5252Ser | TCT | TCT | 0 | TCT | 0 | TCT | 0 | TCA |
| NSP12 | Leu5260Leu | CTT | CTT | 0 | CTT | 0 | CTT | 0 | TTA |
| NSP12 | Leu5282Leu | CTA | CTA | 0 | CTA | 0 | CTA | 0 | TTA |
| NSP12 | Ser5295Ser | TCT | TCT | 0 | TCT | 0 | TCT | 0 | TCA |
| NSP12 | Leu5298Leu | CTT | CTT | 0 | CTT | 0 | CTT | 0 | TTA |
| NSP13 | Leu5330Leu | CTT | CTT | 0 | CTT | 0 | CTT | 0 | TTA |
| NSP13 | Ser5367Ser | TCT | TCT | 0 | TCT | 0 | TCT | 0 | TCA |
| NSP13 | Leu5386Leu | CTT | CTT | 0 | CTT | 0 | CTT | 0 | TTA |
| NSP13 | Ser5392Ser | AGC | AGC | 0 | AGC | 0 | AGC | 0 | TCG |
| NSP13 | Ser5423Ser | AGC | AGC | 0 | AGC | 0 | AGC | 0 | TCG |
| NSP13 | Leu5453Leu | CTC | CTC | 0 | CTC | 0 | CTC | 0 | TTG |
| NSP13 | Leu5455Leu | CTT | CTT | 0 | CTT | 0 | CTT | 0 | TTA |
| NSP13 | Leu5461Leu | CTC | CTC | 0 | CTC | 0 | CTC | 0 | TTG |
| NSP13 | Leu5470Leu | CTG | CTG | 0 | CTG | 0 | CTG | 0 | TTG |
| NSP13 | Ser5471Ser | TCT | TCT | 0 | TCT | 0 | TCT | 0 | TCA |
| NSP13 | Lez5481Leu | CTG | CTG | 0 | CTG | 0 | CTG | 0 | TTG |
| NSP13 | Ser5482Ser | TCT | TCT | 0 | TCT | 0 | TCT | 0 | TCA |
| NSP13 | Leu5488Leu | CTT | CTT | 0 | CTT | 0 | CTT | 0 | TTA |
| NSP13 | Leu5499Leu | CTT | CTT | 0 | CTT | 0 | CTT | 0 | TTA |
| NSP13 | Ser5514Ser | AGT | AGT | 0 | AGT | 0 | AGT | 0 | TCA |
| NSP13 | Leu5550Leu | CTC | CTC | 0 | CTC | 0 | CTC | 0 | TTG |
| NSP13 | Ser5559Ser | AGT | AGT | 0 | AGT | 0 | AGT | 0 | TCA |
| NSP13 | Leu5563Leu | CTA | CTA | 0 | CTA | 0 | CTA | 0 | TTA |
| NSP13 | Leu5579Leu | CTC | CTC | 0 | CTC | 0 | CTC | 0 | TTG |
| NSP13 | Ser5586Ser | TCT | TCT | 0 | TCT | 0 | TCT | 0 | TCA |
| NSP13 | Ser5587Ser | AGC | AGC | 0 | AGC | 0 | AGC | 0 | TCG |
| NSP13 | Ser5601Ser | TCT | TCT | 0 | TCT | 0 | TCT | 0 | TCA |
| NSP13 | Leu5603Leu | CTC | CTC | 0 | CTC | 0 | CTC | 0 | TTA |
| NSP13 | Ser5612Ser | AGT | AGT | 0 | AGT | 0 | AGT | 0 | TCA |
| NSP13 | Leu5618Leu | CTA | CTA | 0 | CTA | 0 | CTA | 0 | TTA |
| NSP13 | Leu5620Leu | CTC | CTC | 0 | CTC | 0 | CTC | 0 | TTG |
| NSP13 | Ser5624Ser | TCT | TCT | 0 | TCT | 0 | TCT | 0 | TCA |
| NSP13 | Ser5633Ser | TCT | TCT | 0 | TCT | 0 | TCT | 0 | TCA |
| NSP13 | Leu5640Leu | CTA | CTA | 0 | CTA | 0 | CTA | 0 | TTA |
| NSP13 | Ser5654Ser | AGT | AGT | 0 | AGT | 0 | AGT | 0 | TCA |
| NSP13 | Ser5708Ser | AGT | AGT | 0 | AGT | 0 | AGT | 0 | TCA |
| NSP13 | Leu5823Leu | CTT | CTT | 0 | CTT | 0 | CTT | 0 | CTT |
| NSP13 | Leu5851Leu | CTA | CTA | 0 | CTA | 0 | CTA | 0 | CTA |
| NSP13 | Ser5878Ser | TCT | TCT | 0 | TCT | 0 | TCT | 0 | TCT |
| NSP13 | Leu5896Leu | CTT | CTT | 0 | CTT | 0 | CTT | 0 | CTT |
| NSP13 | Ser5900Ser | TCT | TCT | 0 | TCT | 0 | TCT | 0 | TCT |
| NSP14 | Leu5904Leu | CTT | CTT | 0 | CTT | 0 | CTT | 0 | CTT |
| NSP14 | Ser5912Ser | AGT | AGT | 0 | AGT | 0 | AGT | 0 | AGT |
| NSP14 | Leu5913Leu | CTT | CTT | 0 | CTT | 0 | CTT | 0 | CTT |
| NSP14 | Leu5931Leu | CTC | CTC | 0 | CTC | 0 | CTC | 0 | CTC |
| NSP14 | Ser5936Ser | AGT | AGT | 0 | AGT | 0 | AGT | 0 | AGT |
| NSP14 | Leu5951Leu | CTC | CTC | 0 | CTC | 0 | CTC | 0 | CTC |
| NSP14 | Ser5952Ser | AGT | AGT | 0 | AGT | 0 | AGT | 0 | AGT |
| NSP14 | Leu5978Leu | CTC | CTC | 0 | CTC | 0 | CTC | 0 | CTC |
| NSP14 | Ser5980Ser | TCT | TCT | 0 | TCT | 0 | TCT | 0 | TCT |
| NSP14 | Leu6033Leu | CTA | CTA | 0 | CTA | 0 | CTA | 0 | CTA |
| NSP14 | Ser6036Ser | TCT | TCT | 0 | TCT | 0 | TCT | 0 | TCT |
| NSP14 | Leu6041Leu | CTA | CTA | 0 | CTA | 0 | CTA | 0 | CTA |
| NSP14 | Ser6058Ser | TCC | TCC | 0 | TCC | 0 | TCC | 0 | TCC |
| NSP14 | Ser6061Ser | AGT | AGT | 0 | AGT | 0 | AGT | 0 | AGT |
| NSP14 | Leu6073Leu | CTC | CTC | 0 | CTC | 0 | CTC | 0 | CTC |
| NSP14 | Leu6076Leu | CTT | CTT | 0 | CTT | 0 | CTT | 0 | CTT |
| NSP14 | Leu6081Leu | CTT | CTT | 0 | CTT | 0 | CTT | 0 | CTT |
| NSP14 | Ser6095Ser | AGT | AGT | 0 | AGT | 0 | AGT | 0 | AGT |
| NSP14 | Leu6098Leu | CTT | CTT | 0 | CTT | 0 | CTT | 0 | CTT |
| NSP14 | Leu6101Leu | CTC | CTC | 0 | CTC | 0 | CTC | 0 | CTC |
| NSP14 | Ser6102Ser | TCT | TCT | 0 | TCT | 0 | TCT | 0 | TCT |
| NSP14 | Ser6118Ser | TCT | TCT | 0 | TCT | 0 | TCT | 0 | TCT |
| NSP14 | Leu6133Leu | CTA | CTA | 0 | CTA | 0 | CTA | 0 | CTA |
| NSP14 | Ser6142Ser | TCC | TCC | 0 | TCC | 0 | TCC | 0 | TCC |
| NSP14 | Ser6154Ser | TCT | TCT | 0 | TCT | 0 | TCT | 0 | TCT |
| NSP14 | Leu6177Leu | CTA | CTA | 0 | CTA | 0 | CTA | 0 | CTA |
| NSP14 | Ser6179Ser | AGC | AGC | 0 | AGC | 0 | AGC | 0 | AGC |
| NSP14 | Leu6183Leu | CTG | CTG | 0 | CTG | 0 | CTG | 0 | CTG |
| NSP14 | Ser6195Ser | AGT | AGT | 0 | AGT | 0 | AGT | 0 | AGT |
| NSP14 | Leu620462 | CTA | CTA | 0 | CTA | 0 | CTA | 0 | CTA |
| NSP14 | Leu6253Leu | CTT | CTT | 0 | CTT | 0 | CTT | 0 | CTT |
| NSP14 | Ser6281Ser | AGT | AGT | 0 | AGT | 0 | AGT | 0 | AGT |
| NSP14 | Ser6293Ser | TCT | TCT | 0 | TCT | 0 | TCT | 0 | TCT |
| NSP14 | Leu6307Leu | CTA | CTA | 0 | CTA | 0 | CTA | 0 | CTA |
| NSP14 | Ser6320Ser | TCC | TCC | 0 | TCC | 0 | TCC | 0 | TCC |
| NSP14 | Leu6330Leu | CTA | CTA | 0 | CTA | 0 | CTA | 0 | CTA |
| NSP14 | Ser6331Ser | TCT | TCT | 0 | TCT | 0 | TCT | 0 | TCT |
| NSP14 | Leu6333Leu | CTT | CTT | 0 | CTT | 0 | CTT | 0 | CTT |
| NSP14 | Ser6342Ser | AGT | AGT | 0 | AGT | 0 | AGT | 0 | AGT |
| NSP14 | Ser6358Ser | AGT | AGT | 0 | AGT | 0 | AGT | 0 | AGT |
| NSP14 | Ser6372Ser | TCT | TCT | 0 | TCT | 0 | TCT | 0 | TCT |
| NSP14 | Ser6374Ser | AGT | AGT | 0 | AGT | 0 | AGT | 0 | AGT |
| NSP14 | Ser6378Ser | TCT | TCT | 0 | TCT | 0 | TCT | 0 | TCT |
| NSP14 | Leu6392Leu | CTA | CTA | 0 | CTA | 0 | CTA | 0 | CTA |
| NSP14 | Ser6394Ser | TCT | TCT | 0 | TCT | 0 | TCT | 0 | TCT |
| NSP14 | Leu6419Leu | CTC | CTC | 0 | CTC | 0 | CTC | 0 | CTC |
| NSP14 | Ser6431Ser | AGC | AGC | 0 | AGC | 0 | AGC | 0 | AGC |
| NSP14 | Leu6443Leu | CTC | CTC | 0 | CTC | 0 | CTC | 0 | CTC |
| NSP14 | Leu6450Leu | CTT | CTT | 0 | CTT | 0 | CTT | 0 | CTT |
| NSP15 | Ser6452Ser | AGT | AGT | 0 | AGT | 0 | AGT | 0 | AGT |
| NSP15 | Ser6476Ser | TCT | TCT | 0 | TCT | 0 | TCT | 0 | TCT |
| NSP15 | Leu6508Leu | CTT | CTT | 0 | CTT | 0 | CTT | 0 | CTT |
| NSP15 | Leu6523Leu | CTC | CTC | 0 | CTC | 0 | CTC | 0 | CTC |
| NSP15 | Ser6548Ser | CTC | CTC | 0 | CTC | 0 | CTC | 0 | CTC |
| NSP15 | Ser6554Ser | TCT | TCT | 0 | TCT | 0 | TCT | 0 | TCT |
| NSP15 | Leu6570Leu | CTC | CTC | 0 | CTC | 0 | CTC | 0 | CTC |
| NSP15 | Leu6593Leu | CTT | CTT | 0 | CTT | 0 | CTT | 0 | CTT |
| NSP15 | Ser6598Ser | AGT | AGT | 0 | AGT | 0 | AGT | 0 | AGT |
| NSP15 | Ser6605Ser | TCT | TCT | 0 | TCT | 0 | TCT | 0 | TCT |
| NSP15 | Ser6612Ser | AGT | AGT | 0 | AGT | 0 | AGT | 0 | AGT |
| NSP15 | Leu6613Leu | CTT | CTT | 0 | CTT | 0 | CTT | 0 | CTT |

| | | | | | | | OTS | |
|---|---|---|---|---|---|---|---|---|
| | | OTS | OTS | | OTS | | Fragment | |
| | | Fragment 7 | Fragment 8 | | Fragment 7/8 | | 4/5/7/8 | |
| Genome | | Nucleo- | | Nucleo- | | Nucleo- | | Nucleo- |
| Anno- | | tide | OTS | tide | OTS | tide | OTS | tide |
| tation | Genome | changes | codons | changes | codons | changes | codons | changes |
| peptide | Changes | 149 | 66 | 120 | 146 | 269 | 337 | 604 |
| NSP1 | Leu46Leu | 0 | CTT | 0 | CTT | 0 | CTT | 0 |
| NSP1 | Leu64Leu | 0 | CTT | 0 | CTT | 0 | CTT | 0 |
| NSP1 | Leu88Leu | 0 | CTG | 0 | CTG | 0 | CTG | 0 |
| NSP1 | Leu92Leu | 0 | CTC | 0 | CTC | 0 | CTC | 0 |
| NSP1 | Ser100Ser | 0 | AGT | 0 | AGT | 0 | AGT | 0 |
| NSP1 | Leu104Leu | 0 | CTT | 0 | CTT | 0 | CTT | 0 |
| NSP1 | Leu107Leu | 0 | CTT | 0 | CTT | 0 | CTT | 0 |
| NSP1 | Leu122Leu | 0 | TTC | 0 | TTC | 0 | TTC | 0 |
| NSP1 | Leu123Leu | 0 | TTC | 0 | TTC | 0 | TTC | 0 |
| NSP1 | Leu140Leu | 0 | CTA | 0 | CTA | 0 | CTA | 0 |
| NSP1 | Leu149Leu | 0 | CTT | 0 | CTT | 0 | CTT | 0 |
| NSP1 | Ser166Ser | 0 | AGC | 0 | AGC | 0 | AGC | 0 |
| NSP1 | Ser167Ser | 0 | AGT | 0 | AGT | 0 | AGT | 0 |
| NSP1 | Leu173Leu | 0 | CTC | 0 | CTC | 0 | CTC | 0 |
| NSP1 | Leu177Leu | 0 | CTT | 0 | CTT | 0 | CTT | 0 |
| NSP2 | Leu198Leu | 0 | CTT | 0 | CTT | 0 | CTT | 0 |
| NSP2 | Leu204Leu | 0 | CTT | 0 | CTT | 0 | CTT | 0 |
| NSP2 | Leu205Leu | 0 | CTA | 0 | CTA | 0 | CTA | 0 |
| NSP2 | Leu219Leu | 0 | CTG | 0 | CTG | 0 | CTG | 0 |
| NSP2 | Ser245Ser | 0 | TCT | 0 | TCT | 0 | TCT | 0 |
| NSP2 | Ser248Ser | 0 | AGC | 0 | AGC | 0 | AGC | 0 |
| NSP2 | Ser279Ser | 0 | TCC | 0 | TCC | 0 | TCC | 0 |
| NSP2 | Leu293Leu | 0 | CTT | 0 | CTT | 0 | CTT | 0 |
| NSP2 | Ser302Ser | 0 | TCT | 0 | TCT | 0 | TCT | 0 |
| NSP2 | Leu320Leu | 0 | CTC | 0 | CTC | 0 | CTC | 0 |
| NSP2 | Ser383Ser | 0 | AGT | 0 | AGT | 0 | AGT | 0 |
| NSP2 | Ser391Ser | 0 | TCT | 0 | TCT | 0 | TCT | 0 |
| NSP2 | Leu397Leu | 0 | CTT | 0 | CTT | 0 | CTT | 0 |
| NSP2 | Ser412Ser | 0 | TCT | 0 | TCT | 0 | TCT | 0 |
| NSP2 | Ser428Ser | 0 | AGC | 0 | AGC | 0 | AGC | 0 |
| NSP2 | Ser443Ser | 0 | TCC | 0 | TCC | 0 | TCC | 0 |
| NSP2 | Leu446Leu | 0 | CTT | 0 | CTT | 0 | CTT | 0 |
| NSP2 | Leu450Leu | 0 | CTT | 0 | CTT | 0 | CTT | 0 |
| NSP2 | Leu451Leu | 0 | CTT | 0 | CTT | 0 | CTT | 0 |
| NSP2 | Leu454Leu | 0 | CTC | 0 | CTC | 0 | CTC | 0 |
| NSP2 | Leu469Leu | 0 | CTT | 0 | CTT | 0 | CTT | 0 |
| NSP2 | Ser479Ser | 0 | TCT | 0 | TCT | 0 | TCT | 0 |
| NSP2 | Ser481Ser | 0 | TCT | 0 | TCT | 0 | TCT | 0 |
| NSP2 | Ser483Ser | 0 | TCC | 0 | TCC | 0 | TCC | 0 |
| NSP2 | Ser485Ser | 0 | AGT | 0 | AGT | 0 | AGT | 0 |
| NSP2 | Ser505Ser | 0 | TCC | 0 | TCC | 0 | TCC | 0 |
| NSP2 | Leu530Leu | 0 | CTG | 0 | CTG | 0 | CTG | 0 |
| NSP2 | Ser531Ser | 0 | AGT | 0 | AGT | 0 | AGT | 0 |
| NSP2 | Leu533Leu | 0 | CTT | 0 | CTT | 0 | CTT | 0 |
| NSP2 | Ser549Ser | 0 | TCC | 0 | TCC | 0 | TCC | 0 |
| NSP2 | Leu552Leu | 0 | CTT | 0 | CTT | 0 | CTT | 0 |
| NSP2 | Ser558Ser | 0 | TCT | 0 | TCT | 0 | TCT | 0 |
| NSP2 | Leu570Leu | 0 | CTA | 0 | CTA | 0 | CTA | 0 |
| NSP2 | Leu578Leu | 0 | CTG | 0 | CTG | 0 | CTG | 0 |
| NSP2 | Leu580Leu | 0 | CTC | 0 | CTC | 0 | CTC | 0 |
| NSP2 | Ser588Ser | 0 | TCT | 0 | TCT | 0 | TCT | 0 |
| NSP2 | Leu595Leu | 0 | CTA | 0 | CTA | 0 | CTA | 0 |
| NSP2 | Leu613Leu | 0 | CTA | 0 | CTA | 0 | CTA | 0 |
| NSP2 | Leu624Leu | 0 | CTC | 0 | CTC | 0 | CTC | 0 |
| NSP2 | Leu628Leu | 0 | CTT | 0 | CTT | 0 | CTT | 0 |
| NSP2 | Leu631Leu | 0 | CTT | 0 | CTT | 0 | CTT | 0 |
| NSP2 | Leu642Leu | 0 | CTT | 0 | CTT | 0 | CTT | 0 |
| NSP2 | Ser674Ser | 0 | AGT | 0 | AGT | 0 | AGT | 0 |
| NSP2 | Leu681Leu | 0 | CTT | 0 | CTT | 0 | CTT | 0 |
| NSP2 | Ser692Ser | 0 | TCT | 0 | TCT | 0 | TCT | 0 |
| NSP2 | Leu700Leu | 0 | CTT | 0 | CTT | 0 | CTT | 0 |
| NSP2 | Ser723Ser | 0 | TCC | 0 | TCC | 0 | TCC | 0 |
| NSP2 | Leu729Leu | 0 | CTA | 0 | CTA | 0 | CTA | 0 |
| NSP2 | Leu730Leu | 0 | CTC | 0 | CTC | 0 | CTC | 0 |
| NSP2 | Leu733Leu | 0 | CTA | 0 | CTA | 0 | CTA | 0 |
| NSP2 | Leu747Leu | 0 | CTT | 0 | CTT | 0 | CTT | 0 |
| NSP2 | Ser771Ser | 0 | AGT | 0 | AGT | 0 | AGT | 0 |
| NSP2 | Leu788Leu | 0 | CTT | 0 | CTT | 0 | CTT | 0 |
| NSP2 | Leu791Leu | 0 | CTC | 0 | CTC | 0 | CTC | 0 |
| NSP2 | Leu815Leu | 0 | CTC | 0 | CTC | 0 | CTC | 0 |
| NSP3 | Ser838Ser | 0 | AGT | 0 | AGT | 0 | AGT | 0 |
| NSP3 | Leu845Leu | 0 | CTT | 0 | CTT | 0 | CTT | 0 |
| NSP3 | Leu853Leu | 0 | CTT | 0 | CTT | 0 | CTT | 0 |
| NSP3 | Ser858Ser | 0 | TCT | 0 | TCT | 0 | TCT | 0 |
| NSP3 | Leu864Leu | 0 | CTC | 0 | CTC | 0 | CTC | 0 |
| NSP3 | Ser887Ser | 0 | TCT | 0 | TCT | 0 | TCT | 0 |
| NSP3 | Leu893Leu | 0 | CTG | 0 | CTG | 0 | CTG | 0 |
| NSP3 | Ser901Ser | 0 | AGT | 0 | AGT | 0 | AGT | 0 |
| NSP3 | Ser911Ser | 0 | TCT | 0 | TCT | 0 | TCT | 0 |
| NSP3 | Ser923Ser | 0 | TCT | 0 | TCT | 0 | TCT | 0 |
| NSP3 | Ser966Ser | 0 | TCT | 0 | TCT | 0 | TCT | 0 |
| NSP3 | Leu969Leu | 0 | CTT | 0 | CTT | 0 | CTT | 0 |
| NSP3 | Ser984Ser | 0 | AGT | 0 | AGT | 0 | AGT | 0 |
| NSP3 | Ser994Ser | 0 | AGT | 0 | AGT | 0 | AGT | 0 |
| NSP3 | Leu1016Leu | 0 | CTT | 0 | CTT | 0 | CTT | 0 |
| NSP3 | Leu1034Leu | 0 | CTT | 0 | CTT | 0 | CTT | 0 |
| NSP3 | Leu1065Leu | 0 | CTT | 0 | CTT | 0 | CTT | 0 |
| NSP3 | Ser1087Ser | 0 | TCT | 0 | TCT | 0 | TCT | 0 |
| NSP3 | Leu1097Leu | 0 | CTT | 0 | CTT | 0 | CTT | 0 |
| NSP3 | Ser1102Ser | 0 | AGT | 0 | AGT | 0 | AGT | 0 |
| NSP3 | Leu1110Leu | 0 | CTT | 0 | CTT | 0 | CTT | 0 |
| NSP3 | Leu1115Leu | 0 | CTT | 0 | CTT | 0 | CTT | 0 |
| NSP3 | Leu1130Leu | 0 | CTT | 0 | CTT | 0 | CTT | 0 |
| NSP3 | Leu1131Leu | 0 | CTT | 0 | CTT | 0 | CTT | 0 |
| NSP3 | Ser1133Ser | 0 | AGT | 0 | AGT | 0 | AGT | 0 |
| NSP3 | Leu1144Leu | 0 | CTA | 0 | CTA | 0 | CTA | 0 |
| NSP3 | Leu1145Leu | 0 | CTT | 0 | CTT | 0 | CTT | 0 |
| NSP3 | Ser1161Ser | 0 | TCT | 0 | TCT | 0 | TCT | 0 |
| NSP3 | Leu1182Leu | 0 | CTC | 0 | CTC | 0 | CTC | 0 |
| NSP3 | Leu1186Leu | 0 | CTT | 0 | CTT | 0 | CTT | 0 |
| NSP3 | Ser1189Ser | 0 | AGC | 0 | AGC | 0 | AGC | 0 |
| NSP3 | Ser1195Ser | 0 | AGT | 0 | AGT | 0 | AGT | 0 |
| NSP3 | Leu1243Leu | 0 | CTG | 0 | CTG | 0 | CTG | 0 |
| NSP3 | Leu1249Leu | 0 | CTC | 0 | CTC | 0 | CTC | 0 |
| NSP3 | Leu1255Leu | 0 | CTT | 0 | CTT | 0 | CTT | 0 |
| NSP3 | Leu1263Leu | 0 | CTT | 0 | CTT | 0 | CTT | 0 |
| NSP3 | Leu1270Leu | 0 | CTT | 0 | CTT | 0 | CTT | 0 |
| NSP3 | Ser1272Ser | 0 | AGT | 0 | AGT | 0 | AGT | 0 |
| NSP3 | Leu1313Leu | 0 | CTA | 0 | CTA | 0 | CTA | 0 |
| NSP3 | Leu1346Leu | 0 | CTT | 0 | CTT | 0 | CTT | 0 |
| NSP3 | Leu1356Leu | 0 | CTA | 0 | CTA | 0 | CTA | 0 |
| NSP3 | Ser1358Ser | 0 | TCT | 0 | TCT | 0 | TCT | 0 |
| NSP3 | Ser1361Ser | 0 | TCT | 0 | TCT | 0 | TCT | 0 |
| NSP3 | Leu1268Leu | 0 | CTT | 0 | CTT | 0 | CTT | 0 |
| NSP3 | Ser1372Ser | 0 | TCT | 0 | TCT | 0 | TCT | 0 |
| NSP3 | Leu1379Leu | 0 | CTT | 0 | CTT | 0 | CTT | 0 |
| NSP3 | Ser1427Ser | 0 | AGT | 0 | AGT | 0 | AGT | 0 |
| NSP3 | Leu1434Leu | 0 | CTT | 0 | CTT | 0 | CTT | 0 |
| NSP3 | Leu1438Leu | 0 | CTT | 0 | CTT | 0 | CTT | 0 |
| NSP3 | Leu1441Leu | 0 | CTA | 0 | CTA | 0 | CTA | 0 |
| NSP3 | Leu1445Leu | 0 | CTT | 0 | CTT | 0 | CTT | 0 |
| NSP3 | Leu1450Leu | 0 | CTT | 0 | CTT | 0 | CTT | 0 |
| NSP3 | Leu1469Leu | 0 | CTC | 0 | CTC | 0 | CTC | 0 |
| NSP3 | Ser1476Ser | 0 | TCT | 0 | TCT | 0 | TCT | 0 |
| NSP3 | Ser1478Ser | 0 | TCT | 0 | TCT | 0 | TCT | 0 |
| NSP3 | Leu1490Leu | 0 | CTT | 0 | CTT | 0 | CTT | 0 |
| NSP3 | Ser1492Ser | 0 | TCT | 0 | TCT | 0 | TCT | 0 |
| NSP3 | Ser1493Ser | 0 | TCT | 0 | TCT | 0 | TCT | 0 |
| NSP3 | Ser1494Ser | 0 | TCT | 0 | TCT | 0 | TCT | 0 |
| NSP3 | Leu1507Leu | 0 | CTT | 0 | CTT | 0 | CTT | 0 |
| NSP3 | Ser1510Ser | 0 | TCC | 0 | TCC | 0 | TCC | 0 |
| NSP3 | Ser1515Ser | 0 | TCC | 0 | TCC | 0 | TCC | 0 |
| NSP3 | Ser1517Ser | 0 | TCT | 0 | TCT | 0 | TCT | 0 |
| NSP3 | Ser1520Ser | 0 | TCT | 0 | TCT | 0 | TCT | 0 |
| NSP3 | Leu1528Leu | 0 | CTT | 0 | CTT | 0 | CTT | 0 |
| NSP3 | Ser1534Ser | 0 | AGT | 0 | AGT | 0 | AGT | 0 |
| NSP3 | Ser1539Ser | 0 | AGT | 0 | AGT | 0 | AGT | 0 |
| NSP3 | Leu1546Leu | 0 | CTA | 0 | CTA | 0 | CTA | 0 |
| NSP3 | Leu1556Leu | 0 | CTT | 0 | CTT | 0 | CTT | 0 |
| NSP3 | Leu1559Leu | 0 | CTT | 0 | CTT | 0 | CTT | 0 |
| NSP3 | Leu1560Leu | 0 | CTT | 0 | CTT | 0 | CTT | 0 |
| NSP3 | Ser1561Ser | 0 | TCT | 0 | TCT | 0 | TCT | 0 |
| NSP3 | Leu1579Leu | 0 | TTG | 0 | TTG | 0 | TTG | 0 |
| NSP3 | Leu1627Leu | 0 | CTA | 0 | CTA | 0 | CTA | 0 |
| NSP3 | Ser1641Ser | 0 | AGT | 0 | AGT | 0 | AGT | 0 |
| NSP3 | Leu1643Leu | 0 | CTG | 0 | CTG | 0 | CTG | 0 |
| NSP3 | Ser1666Ser | 0 | TCT | 0 | TCT | 0 | TCT | 0 |
| NSP3 | Leu1676Leu | 0 | CTT | 0 | CTT | 0 | CTT | 0 |
| NSP3 | Leu1683Leu | 0 | CTC | 0 | CTC | 0 | CTC | 0 |
| NSP3 | Leu1695Leu | 0 | CTA | 0 | CTA | 0 | CTA | 0 |
| NSP3 | Leu1713Leu | 0 | CTT | 0 | CTT | 0 | CTT | 0 |
| NSP3 | Ser1733Ser | 0 | AGT | 0 | AGT | 0 | AGT | 0 |
| NSP3 | Ser1743Ser | 0 | TCT | 0 | TCT | 0 | TCT | 0 |
| NSP3 | Leu1762Leu | 0 | CTT | 0 | CTT | 0 | CTT | 0 |
| NSP3 | Leu1774Leu | 0 | CTT | 0 | CTT | 0 | CTT | 0 |
| NSP3 | Ser1775Ser | 0 | TCT | 0 | TCT | 0 | TCT | 0 |
| NSP3 | Leu1797Leu | 0 | CTA | 0 | CTA | 0 | CTA | 0 |
| NSP3 | Leu1816Leu | 0 | CTT | 0 | CTT | 0 | CTT | 0 |
| NSP3 | Ser1825Ser | 0 | AGT | 0 | AGT | 0 | AGT | 0 |
| NSP3 | Ser1841Ser | 0 | TCT | 0 | TCT | 0 | TCT | 0 |
| NSP3 | Leu1853Leu | 0 | CTT | 0 | CTT | 0 | CTT | 0 |
| NSP3 | Ser2856Ser | 0 | TCC | 0 | TCC | 0 | TCC | 0 |
| NSP3 | Ser1872Ser | 0 | AGT | 0 | AGT | 0 | AGT | 0 |
| NSP3 | Ser1905Ser | 0 | TCT | 0 | TCT | 0 | TCT | 0 |
| NSP3 | Leu2028Leu | 0 | CTG | 0 | CTG | 0 | TTG | 1 |
| NSP3 | Leu2039Leu | 0 | CTT | 0 | CTT | 0 | TTA | 2 |
| NSP3 | Leu2044Leu | 0 | CTA | 0 | CTA | 0 | TTA | 1 |
| NSP3 | Ser2048Ser | 0 | TCT | 0 | TCT | 0 | TCA | 1 |
| NSP3 | Leu2062Leu | 0 | CTT | 0 | CTT | 0 | TTA | 2 |
| NSP3 | Leu2077Leu | 0 | CTT | 0 | CTT | 0 | TTA | 2 |
| NSP3 | Ser2083Ser | 0 | AGT | 0 | AGT | 0 | TCA | 3 |
| NSP3 | Leu2095Leu | 0 | TTA | 0 | TTA | 0 | CTA | 1 |
| NSP3 | Ser2103Ser | 0 | TCT | 0 | TCT | 0 | TCA | 1 |
| NSP3 | Ser2104Ser | 0 | AGT | 0 | AGT | 0 | TCA | 3 |
| NSP3 | Ser2114Ser | 0 | TCT | 0 | TCT | 0 | TCA | 1 |
| NSP3 | Leu2122Leu | 0 | CTT | 0 | CTT | 0 | TTA | 2 |
| NSP3 | Ser2132Ser | 0 | AGT | 0 | AGT | 0 | TCA | 3 |
| NSP3 | Leu2146Leu | 0 | CTT | 0 | CTT | 0 | TTA | 2 |
| NSP3 | Ser2151Ser | 0 | AGT | 0 | AGT | 0 | TCA | 3 |
| NSP3 | Leu2177Leu | 0 | CTA | 0 | CTA | 0 | TTA | 1 |
| NSP3 | Ser2185Ser | 0 | AGT | 0 | AGT | 0 | TCA | 3 |
| NSP3 | Ser2188Ser | 0 | TCT | 0 | TCT | 0 | TCA | 1 |
| NSP3 | Ser2193Ser | 0 | TCT | 0 | TCT | 0 | TCA | 1 |
| NSP3 | Ser2205Ser | 0 | AGT | 0 | AGT | 0 | TCA | 3 |
| NSP3 | Leu2211Leu | 0 | CTA | 0 | CTA | 0 | TTA | 1 |
| NSP3 | Ser2224Ser | 0 | TCT | 0 | TCT | 0 | TCA | 1 |
| NSP3 | Leu2226Leu | 0 | CTG | 0 | CTG | 0 | TTG | 1 |
| NSP3 | Leu2235Leu | 0 | CTA | 0 | CTA | 0 | TTA | 1 |
| NSP3 | Ser2237Ser | 0 | AGT | 0 | AGT | 0 | TCA | 3 |
| NSP3 | Leu2240Leu | 0 | CTA | 0 | CTA | 0 | TTA | 1 |
| NSP3 | Ser2242Ser | 0 | TCT | 0 | TCT | 0 | TCA | 1 |
| NSP3 | Ser2255Ser | 0 | TCT | 0 | TCT | 0 | TCA | 1 |
| NSP3 | Ser2261Ser | 0 | TCT | 0 | TCT | 0 | TCA | 1 |
| NSP3 | Ser2273Ser | 0 | TCT | 0 | TCT | 0 | TCA | 1 |
| NSP3 | Ser2285Ser | 0 | TCT | 0 | TCT | 0 | TCA | 1 |
| NSP3 | Ser2289Ser | 0 | AGT | 0 | AGT | 0 | TCA | 3 |
| NSP3 | Leu2292Leu | 0 | CTT | 0 | CTT | 0 | TTA | 2 |
| NSP3 | Ser2293Ser | 0 | AGT | 0 | AGT | 0 | TCA | 3 |
| NSP3 | Ser2297Ser | 0 | TCT | 0 | TCT | 0 | TCA | 1 |
| NSP3 | Ser3303Ser | 0 | TCT | 0 | TCT | 0 | TCA | 1 |
| NSP3 | Ser2313Ser | 0 | TCT | 0 | TCT | 0 | TCA | 1 |
| NSP3 | Leu2333Leu | 0 | CTT | 0 | CTT | 0 | TTA | 2 |
| NSP3 | Leu2341Leu | 0 | CTT | 0 | CTT | 0 | TTA | 2 |
| NSP3 | Ser2352Ser | 0 | AGC | 0 | AGC | 0 | TCG | 3 |
| NSP3 | Ser2360Ser | 0 | AGT | 0 | AGT | 0 | TCA | 3 |
| NSP3 | Ser2362Ser | 0 | TCT | 0 | TCT | 0 | TCA | 1 |
| NSP3 | Leu2364Leu | 0 | CTT | 0 | CTT | 0 | TTA | 2 |
| NSP3 | Leu2371Leu | 0 | CTT | 0 | CTT | 0 | TTA | 2 |
| NSP3 | Ser2396Ser | 0 | AGT | 0 | AGT | 0 | TCA | 3 |
| NSP3 | Ser2433Ser | 0 | TCC | 0 | TCC | 0 | TCG | 1 |
| NSP3 | Leu2447Leu | 0 | CTA | 0 | CTA | 0 | TTA | 1 |
| NSP3 | Ser2462Ser | 0 | AGT | 0 | AGT | 0 | TCA | 3 |
| NSP3 | Ser2466Ser | 0 | AGT | 0 | AGT | 0 | TCA | 3 |
| NSP3 | Leu2475Leu | 0 | CTA | 0 | CTA | 0 | TTA | 1 |
| NSP3 | Ser2487Ser | 0 | TCT | 0 | TCT | 0 | TCA | 1 |
| NSP3 | Ser2488Ser | 0 | TCT | 0 | TCT | 0 | TCA | 1 |
| NSP3 | Ser2493Ser | 0 | AGT | 0 | AGT | 0 | TCA | 3 |
| NSP3 | Ser2500Ser | 0 | TCC | 0 | TCC | 0 | TCG | 1 |
| NSP3 | Leu2503Leu | 0 | CTT | 0 | CTT | 0 | TTA | 2 |
| NSP3 | Ser2517Ser | 0 | TCT | 0 | TCT | 0 | TCA | 1 |
| NSP3 | Leu2518Leu | 0 | CTC | 0 | CTC | 0 | TTG | 2 |
| NSP3 | Ser2519Ser | 0 | TCT | 0 | TCT | 0 | TCA | 1 |
| NSP3 | Leu2527Leu | 0 | CTG | 0 | CTG | 0 | TTG | 1 |
| NSP3 | Ser2553Ser | 0 | TCT | 0 | TCT | 0 | TCA | 1 |
| NSP3 | Ser2558Ser | 0 | TCT | 0 | TCT | 0 | TCA | 1 |
| NSP3 | Leu2564Leu | 0 | CTT | 0 | CTT | 0 | TTA | 2 |
| NSP3 | Leu2570Leu | 0 | CTG | 0 | CTG | 0 | TTG | 1 |
| NSP3 | Leu2572Leu | 0 | CTA | 0 | CTA | 0 | TTA | 1 |
| NSP3 | Ser2578Ser | 0 | TCT | 0 | TCT | 0 | TCA | 1 |
| NSP3 | Ser2583Ser | 0 | AGT | 0 | AGT | 0 | TCA | 3 |
| NSP3 | Leu2609Leu | 0 | CTC | 0 | CTC | 0 | TTG | 2 |
| NSP3 | Leu2612Leu | 0 | CTA | 0 | CTA | 0 | TTA | 1 |
| NSP3 | Leu2620Leu | 0 | CTT | 0 | CTT | 0 | TTA | 2 |
| NSP3 | Ser2625Ser | 0 | TCC | 0 | TCC | 0 | TCG | 1 |
| NSP3 | Ser2631Ser | 0 | TCT | 0 | TCT | 0 | TCA | 1 |
| NSP3 | Leu2655Leu | 0 | CTT | 0 | CTT | 0 | TTA | 2 |
| NSP3 | Ser2661Ser | 0 | TCT | 0 | TCT | 0 | TCA | 1 |
| NSP3 | Ser2669Ser | 0 | AGT | 0 | AGT | 0 | TCA | 3 |
| NSP3 | Leu2675Leu | 0 | CTC | 0 | CTC | 0 | TTG | 2 |
| NSP3 | Leu2688Leu | 0 | CTT | 0 | CTT | 0 | TTA | 2 |
| NSP3 | Ser2695Ser | 0 | AGT | 0 | AGT | 0 | TCA | 3 |
| NSP3 | Ser2706Ser | 0 | AGT | 0 | AGT | 0 | TCA | 3 |
| NSP3 | Ser2722Ser | 0 | TCT | 0 | TCT | 0 | TCA | 1 |
| NSP3 | Leu2725Leu | 0 | CTA | 0 | CTA | 0 | TTA | 1 |
| NSP3 | Ser2731Ser | 0 | AGT | 0 | AGT | 0 | TCA | 3 |
| NSP3 | Leu2760Leu | 0 | CTT | 0 | CTT | 0 | TTA | 2 |
| NSP4 | Leu2778Leu | 0 | CTT | 0 | CTT | 0 | TTA | 2 |
| NSP4 | Leu2781Leu | 0 | CTT | 0 | CTT | 0 | TTA | 2 |
| NSP4 | Ser2797Ser | 0 | TCT | 0 | TCT | 0 | TCA | 1 |
| NSP4 | Ser2804Ser | 0 | AGT | 0 | AGT | 0 | TCA | 3 |
| NSP4 | Ser2822Ser | 0 | TCT | 0 | TCT | 0 | TCA | 1 |
| NSP4 | Ser2839Ser | 0 | AGC | 0 | AGC | 0 | TCG | 3 |
| NSP4 | Ser2844Ser | 0 | AGT | 0 | AGT | 0 | TCA | 3 |
| NSP4 | Ser2890Ser | 0 | AGT | 0 | AGT | 0 | TCA | 3 |
| NSP4 | Leu2902Leu | 0 | CTT | 0 | CTT | 0 | TTA | 2 |
| NSP4 | Ser2926Ser | 0 | TCT | 0 | TCT | 0 | TCA | 1 |
| NSP4 | Leu2939Leu | 0 | CTA | 0 | CTA | 0 | TTA | 1 |
| NSP4 | Ser2942Ser | 0 | TCT | 0 | TCT | 0 | TCA | 1 |
| NSP4 | Ser2947Ser | 0 | AGT | 0 | AGT | 0 | TCA | 3 |
| NSP4 | Leu2956Leu | 0 | CTC | 0 | CTC | 0 | TTG | 2 |
| NSP4 | Ser2960Ser | 0 | TCT | 0 | TCT | 0 | TCA | 1 |
| NSP4 | Leu2969Leu | 0 | CTT | 0 | CTT | 0 | TTA | 2 |
| NSP4 | Ser2972Ser | 0 | TCT | 0 | TCT | 0 | TCA | 1 |
| NSP4 | Ser2981Ser | 0 | TCT | 0 | TCT | 0 | TCA | 1 |
| NSP4 | Ser2999Ser | 0 | TCT | 0 | TCT | 0 | TCA | 1 |
| NSP4 | Ser3001Ser | 0 | AGT | 0 | AGT | 0 | TCA | 3 |
| NSP4 | Leu3006Leu | 0 | CTT | 0 | CTT | 0 | TTA | 2 |
| NSP4 | Ser3013Ser | 0 | TCT | 0 | TCT | 0 | TCA | 1 |
| NSP4 | Leu3027Leu | 0 | CTT | 0 | CTT | 0 | TTA | 2 |
| NSP4 | Leu3034Leu | 0 | CTA | 0 | CTA | 0 | TTA | 1 |
| NSP4 | Ser3046Ser | 0 | TCT | 0 | TCT | 0 | TCA | 1 |
| NSP4 | Leu3060Leu | 0 | CTT | 0 | CTT | 0 | TTA | 2 |
| NSP4 | Ser3075Ser | 0 | AGT | 0 | AGT | 0 | TCA | 3 |
| NSP4 | Leu3084Leu | 0 | CTA | 0 | CTA | 0 | TTA | 1 |
| NSP4 | Leu3086Leu | 0 | CTT | 0 | CTT | 0 | TTA | 2 |
| NSP4 | Leu3092Leu | 0 | CTC | 0 | CTC | 0 | TGG | 2 |
| NSP4 | Ser3106Ser | 0 | TCT | 0 | TCT | 0 | TCA | 1 |
| NSP4 | Leu3116Leu | 0 | CTT | 0 | CTT | 0 | TTA | 2 |
| NSP4 | Ser3121Ser | 0 | TCT | 0 | TCT | 0 | TCA | 1 |
| NSP4 | Ser3149Ser | 0 | TCC | 0 | TCC | 0 | TCG | 1 |
| NSP4 | Ser3158Ser | 0 | AGT | 0 | AGT | 0 | TCA | 3 |
| NSP4 | Leu3161Leu | 0 | CTA | 0 | CTA | 0 | TTA | 1 |
| NSP4 | Ser3171Ser | 0 | TCC | 0 | TCC | 0 | TCG | 1 |
| NSP4 | Ser3173Ser | 0 | AGT | 0 | AGT | 0 | TCA | 2 |
| NSP4 | Leu3180Leu | 0 | CTG | 0 | CTG | 0 | TTG | 1 |
| NSP4 | Leu3191LEu | 0 | CTA | 0 | CTA | 0 | TTA | 1 |
| NSP4 | Ser3195Ser | 0 | AGT | 0 | AGT | 0 | TCA | 3 |
| NSP4 | Leu3198Leu | 0 | CTA | 0 | CTA | 0 | TTA | 1 |
| NSP4 | Leu3201Leu | 0 | CTT | 0 | CTT | 0 | TTA | 2 |
| NSP4 | Leu3210Leu | 0 | CTT | 0 | CTT | 0 | TTA | 2 |
| NSP4 | Ser3218Ser | 0 | AGT | 0 | AGT | 0 | TCA | 3 |
| NSP4 | Ser3225Ser | 0 | AGC | 0 | AGC | 0 | TCG | 3 |
| NSP4 | Leu3234Leu | 0 | CTC | 0 | CTC | 0 | TTG | 2 |
| NSP4 | Leu3238Leu | 0 | CTC | 0 | CTC | 0 | TTG | 2 |
| NSP4 | Ser3242Ser | 0 | AGT | 0 | AGT | 0 | TCA | 3 |
| NSP4 | Ser3246Ser | 0 | TCT | 0 | TCT | 0 | TCA | 1 |
| NSP4 | Leu3249Leu | 0 | CTT | 0 | CTT | 0 | TTA | 2 |
| NSP4 | Ser3256Ser | 0 | TCT | 0 | TCT | 0 | TCA | 1 |
| NSP5 | Ser3264Ser | 0 | AGT | 0 | AGT | 0 | TCA | 3 |
| NSP5 | Ser3273Ser | 0 | TCT | 0 | TCT | 0 | TCA | 1 |
| NSP5 | Leu3290Leu | 0 | CTT | 0 | CTT | 0 | TTA | 2 |
| NSP5 | Leu3293Leu | 0 | CTT | 0 | CTT | 0 | TTA | 2 |
| NSP5 | Leu3295Leu | 0 | CTT | 0 | CTT | 0 | TTA | 2 |
| NSP5 | Ser3309Ser | 0 | TCT | 0 | TCT | 0 | TCA | 1 |
| NSP5 | Leu3313Leu | 0 | CTT | 0 | CTT | 0 | TTA | 2 |
| NSP5 | Leu3321Leu | 0 | CTC | 0 | CTC | 0 | TTG | 2 |
| NSP5 | Ser3325Ser | 0 | TCT | 0 | TCT | 0 | TCA | 1 |
| NSP5 | Leu3338Leu | 0 | CTC | 0 | CTC | 0 | TTG | 2 |
| NSP5 | Ser3344Ser | 0 | TCT | 0 | TCT | 0 | TCA | 1 |
| NSP5 | Leu3350Leu | 0 | CTT | 0 | CTT | 0 | TTA | 2 |
| NSP5 | Leu3352Leu | 0 | CTT | 0 | CTT | 0 | TTA | 2 |
| NSP5 | Ser3386Ser | 0 | TCT | 0 | TCT | 0 | TCA | 1 |
| NSP5 | Leu3404Leu | 0 | TTA | 0 | TTA | 0 | CTT | 2 |
| NSP5 | Ser3410Ser | 0 | AGT | 0 | AGT | 0 | TCA | 3 |
| NSP5 | Ser3421Ser | 0 | TCT | 0 | TCT | 0 | TCA | 1 |
| NSP5 | Leu3483Leu | 0 | CTC | 0 | CTC | 0 | TTG | 2 |
| NSP5 | Leu3490Leu | 0 | CTT | 0 | CTT | 0 | TTA | 2 |
| NSP5 | Leu3495Leu | 0 | CTT | 0 | CTT | 0 | TTA | 2 |
| NSP5 | Leu3505Leu | 0 | CTA | 0 | CTA | 0 | TTA | 1 |
| NSP5 | Leu3513Leu | 0 | CTA | 0 | CTA | 0 | TTA | 1 |
| NSP5 | Leu3516Leu | 0 | CTT | 0 | CTT | 0 | TTA | 2 |
| NSP5 | Leu3535Leu | 0 | CTG | 0 | CTG | 0 | TTG | 1 |
| NSP5 | Ser3547Ser | 0 | AGT | 0 | AGT | 0 | TCA | 3 |
| NSP6 | Ser3570Ser | 0 | AGT | 0 | AGT | 0 | TCA | 3 |
| NSP6 | Leu3585Leu | 0 | CTC | 0 | CTC | 0 | TTG | 2 |
| NSP6 | Leu3581Leu | 0 | CTT | 0 | CTT | 0 | TTA | 2 |
| NSP6 | Ser3597Ser | 0 | AGT | 0 | AGT | 0 | TCA | 3 |
| NSP6 | Ser3601Ser | 0 | TCT | 0 | TCT | 0 | TCA | 1 |
| NSP6 | Ser3622Ser | 0 | TCT | 0 | TCT | 0 | TCA | 1 |
| NSP6 | Leu3636Leu | 0 | CTC | 0 | CTC | 0 | TTG | 2 |
| NSP6 | Ser3643Ser | 0 | TCT | 0 | TCT | 0 | TCA | 1 |
| NSP6 | Leu3644Leu | 0 | CTT | 0 | CTT | 0 | TTA | 2 |
| NSP6 | Ser3658Ser | 0 | AGT | 0 | AGT | 0 | TCA | 3 |
| NSP6 | Ser3673Ser | 0 | AGT | 0 | AGT | 0 | TCA | 3 |
| NSP6 | Ser3675Ser | 0 | TCT | 0 | TCT | 0 | TCA | 1 |
| NSP6 | Leu3679Leu | 0 | CTA | 0 | CTA | 0 | TTA | 1 |
| NSP6 | Leu3692Leu | 0 | CTA | 0 | CTA | 0 | TTA | 1 |
| NSP6 | Leu3694Leu | 0 | CTT | 0 | CTT | 0 | TTA | 2 |
| NSP6 | Leu3711Leu | 0 | CTT | 0 | CTT | 0 | TTA | 2 |
| NSP6 | Leu3717Leu | 0 | CTC | 0 | CTC | 0 | TTG | 2 |
| NSP6 | Ser3732Ser | 0 | TCC | 0 | TCC | 0 | TCG | 1 |
| NSP6 | Leu3736Leu | 0 | CTT | 0 | CTT | 0 | TTA | 2 |
| NSP6 | Ser3739Ser | 0 | TCT | 0 | TCT | 0 | TCA | 1 |
| NSP6 | Ser3742Ser | 0 | TCT | 0 | TCT | 0 | TCA | 1 |
| NSP6 | Leu3776Leu | 0 | CTT | 0 | CTT | 0 | TTA | 2 |
| NSP6 | Leu3781Leu | 0 | CTA | 0 | CTA | 0 | TTA | 2 |
| NSP6 | Leu3796Leu | 0 | CTC | 0 | CTC | 0 | TTG | 2 |
| NSP6 | Leu3828Leu | 0 | CTA | 0 | CTA | 0 | TTA | 1 |
| NSP6 | Leu3829Leu | 0 | CTC | 0 | CTC | 0 | TTG | 2 |
| NSP6 | Ser3834Ser | 0 | AGC | 0 | AGC | 0 | TCG | 3 |
| NSP6 | Leu3840Leu | 0 | CTC | 0 | CTC | 0 | TTG | 2 |
| NSP7 | Ser3860Ser | 0 | TCT | 0 | TCT | 0 | TCA | 1 |
| NSP7 | Leu3873Leu | 0 | CTC | 0 | CTC | 0 | TTG | 2 |
| NSP7 | Leu3879Leu | 0 | CTC | 0 | CTC | 0 | TTG | 2 |
| NSP7 | Ser3885Ser | 0 | TCT | 0 | TCT | 0 | TCA | 1 |
| NSP12 | Leu4792Leu | 2 | CTT | 0 | TTA | 2 | TTA | 2 |
| NSP12 | Ser4816Ser | 1 | TCT | 0 | TCA | 1 | TCA | 1 |
| NSP12 | Ser4824Ser | 3 | AGT | 0 | TCA | 3 | TCA | 3 |
| NSP12 | Ser4825Ser | 1 | TCT | 0 | TCA | 1 | TCA | 1 |
| NSP12 | Ser4842Ser | 3 | AGC | 0 | TCG | 3 | TCG | 3 |
| NSP12 | Leu4851Leu | 1 | CTA | 0 | TTA | 1 | TTA | 1 |
| NSP12 | Leu4860Leu | 1 | CTA | 0 | TTA | 1 | TTA | 1 |
| NSP12 | Leu4861Leu | 1 | CTA | 0 | TTA | 1 | TTA | 1 |
| NSP12 | Leu4889Leu | 1 | CTA | 0 | TTA | 1 | TTA | 1 |
| NSP12 | Leu4905Leu | 2 | CTT | 0 | TTA | 2 | TTA | 2 |
| NSP12 | Ser4911Ser | 3 | AGT | 0 | TCA | 3 | TCA | 3 |
| NSP12 | Leu4918Leu | 2 | CTT | 0 | TTA | 2 | TTA | 2 |
| NSP12 | Leu4935Leu | 2 | CTT | 0 | TTA | 2 | TTA | 2 |
| NSP12 | Ser4940Ser | 3 | AGT | 0 | TCA | 3 | TCA | 3 |
| NSP12 | Ser4952Ser | 1 | TCT | 0 | TCA | 1 | TCA | 1 |
| NSP12 | Ser4955Ser | 3 | AGT | 0 | TCA | 3 | TCA | 3 |
| NSP12 | Ser4983Ser | 3 | AGC | 0 | TCG | 3 | TCG | 3 |
| NSP12 | Ser4998Ser | 3 | AGT | 0 | TCA | 3 | TCA | 3 |
| NSP12 | Leu5005Leu | 2 | CTT | 0 | TTA | 2 | TTA | 2 |
| NSP12 | Leu5021Leu | 2 | CTT | 0 | TTA | 2 | TTA | 2 |
| NSP12 | Leu5027Leu | 2 | CTT | 0 | TTA | 2 | TTA | 2 |
| NSP12 | Leu5029Leu | 2 | CTT | 0 | TTA | 2 | TTA | 2 |
| NSP12 | Ser5038Ser | 3 | AGC | 0 | TCG | 3 | TCG | 3 |
| NSP12 | Ser5055Ser | 3 | AGT | 0 | TCA | 3 | TCA | 3 |
| NSP12 | Leu5064Leu | 1 | CTA | 0 | TTA | 1 | TTA | 1 |
| NSP12 | Ser5083Ser | 3 | AGT | 0 | TCA | 3 | TCA | 3 |
| NSP12 | Leu5098Leu | 2 | CTT | 0 | TTA | 2 | TTA | 2 |
| NSP12 | Ser5100Ser | 1 | TCT | 0 | TCA | 1 | TCA | 1 |
| NSP12 | Leu5118Leu | 2 | CTT | 0 | TTA | 2 | TTA | 2 |
| NSP12 | Leu5122Leu | 2 | CTC | 0 | TTG | 2 | TTG | 2 |
| NSP12 | Leu5149Leu | 2 | CTC | 0 | TTG | 2 | TTG | 2 |
| NSP12 | Ser5150Ser | 1 | TCT | 0 | TCA | 1 | TCA | 1 |
| NSP12 | Ser5159Ser | 3 | AGC | 0 | TCG | 3 | TCG | 3 |
| NSP12 | Ser5163Ser | 1 | TCT | 0 | TCA | 1 | TCA | 1 |
| NSP12 | Leu5166Leu | 1 | CTA | 0 | TTA | 1 | TTA | 1 |
| NSP12 | Ser5169Ser | 3 | AGC | 0 | TCG | 3 | TCG | 3 |
| NSP12 | Leu5177Leu | 2 | CTT | 0 | TTA | 2 | TTA | 2 |
| NSP12 | Ser5186Ser | 1 | TCT | 0 | TCA | 1 | TCA | 1 |
| NSP12 | Leu5196Leu | 2 | CTT | 0 | TTA | 2 | TTA | 2 |
| NSP12 | Ser5205Ser | 1 | TCT | 0 | TCA | 1 | TCA | 1 |
| NSP12 | Leu5210Leu | 1 | CTA | 0 | TTA | 1 | TTA | 1 |
| NSP12 | Leu5220Leu | 2 | CTT | 0 | TTA | 2 | TTA | 2 |
| NSP12 | Leu5229Leu | 1 | CTA | 0 | TTA | 1 | TTA | 1 |
| NSP12 | Leu5245Leu | 2 | CTT | 0 | TTA | 2 | TTA | 2 |
| NSP12 | Ser5252Ser | 1 | TCT | 0 | TCA | 1 | TCA | 1 |
| NSP12 | Leu5260Leu | 2 | CTT | 0 | TTA | 2 | TTA | 2 |
| NSP12 | Leu5282Leu | 1 | CTA | 0 | TTA | 1 | TTA | 1 |
| NSP12 | Ser5295Ser | 1 | TCT | 0 | TCA | 1 | TCA | 1 |
| NSP12 | Leu5298Leu | 2 | CTT | 0 | TTA | 2 | TTA | 2 |
| NSP13 | Leu5330Leu | 2 | CTT | 0 | TTA | 2 | TTA | 2 |
| NSP13 | Ser5367Ser | 1 | TCT | 0 | TCA | 1 | TCA | 1 |
| NSP13 | Leu5386Leu | 2 | CTT | 0 | TTA | 2 | TTA | 2 |
| NSP13 | Ser5392Ser | 3 | AGC | 0 | TCG | 3 | TCG | 3 |
| NSP13 | Ser5423Ser | 3 | AGC | 0 | TCG | 3 | TCG | 3 |
| NSP13 | Leu5453Leu | 2 | CTC | 0 | TTG | 2 | TTG | 2 |
| NSP13 | Leu5455Leu | 2 | CTT | 0 | TTA | 2 | TTA | 2 |
| NSP13 | Leu5461Leu | 2 | CTC | 0 | TTG | 2 | TTG | 2 |
| NSP13 | Leu5470Leu | 1 | CTG | 0 | TTG | 1 | TTG | 1 |
| NSP13 | Ser5471Ser | 1 | TCT | 0 | TCA | 1 | TCA | 1 |
| NSP13 | Lez5481Leu | 1 | CTG | 0 | TTG | 1 | TTG | 1 |
| NSP13 | Ser5482Ser | 1 | TCT | 0 | TCA | 1 | TCA | 1 |
| NSP13 | Leu5488Leu | 2 | CTT | 0 | TTA | 2 | TTA | 2 |
| NSP13 | Leu5499Leu | 2 | CTT | 0 | TTA | 2 | TTA | 2 |
| NSP13 | Ser5514Ser | 3 | AGT | 0 | TCA | 3 | TCA | 3 |
| NSP13 | Leu5550Leu | 2 | CTC | 0 | TTG | 2 | TTG | 2 |
| NSP13 | Ser5559Ser | 3 | AGT | 0 | TCA | 3 | TCA | 3 |
| NSP13 | Leu5563Leu | 1 | CTA | 0 | TTA | 1 | TTA | 1 |
| NSP13 | Leu5579Leu | 2 | CTC | 0 | TTG | 2 | TTG | 2 |
| NSP13 | Ser5586Ser | 1 | TCT | 0 | TCA | 1 | TCA | 1 |
| NSP13 | Ser5587Ser | 3 | AGC | 0 | TCG | 3 | TCG | 3 |
| NSP13 | Ser5601Ser | 1 | TCT | 0 | TCA | 1 | TCA | 1 |
| NSP13 | Leu5603Leu | 2 | CTC | 0 | TTA | 2 | TTA | 2 |
| NSP13 | Ser5612Ser | 3 | AGT | 0 | TCA | 3 | TCA | 3 |
| NSP13 | Leu5618Leu | 1 | CTA | 0 | TTA | 1 | TTA | 1 |
| NSP13 | Leu5620Leu | 2 | CTC | 0 | TTG | 2 | TTG | 2 |
| NSP13 | Ser5624Ser | 1 | TCT | 0 | TCA | 1 | TCA | 1 |
| NSP13 | Ser5633Ser | 1 | TCT | 0 | TCA | 1 | TCA | 1 |
| NSP13 | Leu5640Leu | 1 | CTA | 0 | TTA | 1 | TTA | 1 |
| NSP13 | Ser5654Ser | 3 | AGT | 0 | TCA | 3 | TCA | 3 |
| NSP13 | Ser5708Ser | 3 | AGT | 0 | TCA | 3 | TCA | 3 |
| NSP13 | Leu5823Leu | 0 | TTA | 2 | TTA | 2 | TTA | 2 |
| NSP13 | Leu5851Leu | 0 | TTA | 1 | TTA | 1 | TTA | 1 |
| NSP13 | Ser5878Ser | 0 | TCA | 1 | TCA | 1 | TCA | 1 |
| NSP13 | Leu5896Leu | 0 | TTA | 2 | TTA | 2 | TTA | 2 |
| NSP13 | Ser5900Ser | 0 | TCA | 1 | TCA | 1 | TCA | 1 |
| NSP14 | Leu5904Leu | 0 | TTA | 2 | TTA | 2 | TTA | 2 |
| NSP14 | Ser5912Ser | 0 | TCA | 3 | TCA | 3 | TCA | 3 |
| NSP14 | Leu5913Leu | 0 | TTA | 2 | TTA | 2 | TTA | 2 |
| NSP14 | Leu5931Leu | 0 | TTG | 2 | TTG | 2 | TTG | 2 |
| NSP14 | Ser5936Ser | 0 | TCA | 3 | TCA | 3 | TCA | 3 |
| NSP14 | Leu5951Leu | 0 | TTG | 2 | TTG | 2 | TTG | 2 |
| NSP14 | Ser5952Ser | 0 | TCA | 3 | TCA | 3 | TCA | 3 |
| NSP14 | Leu5978Leu | 0 | TTG | 2 | TTG | 2 | TTG | 2 |
| NSP14 | Ser5980Ser | 0 | TCA | 1 | TCA | 1 | TCA | 1 |
| NSP14 | Leu6033Leu | 0 | TTA | 1 | TTA | 1 | TTA | 1 |
| NSP14 | Ser6036Ser | 0 | TCA | 1 | TCA | 1 | TCA | 1 |
| NSP14 | Leu6041Leu | 0 | TTA | 1 | TTA | 1 | TTA | 1 |
| NSP14 | Ser6058Ser | 0 | TCG | 1 | TCG | 1 | TCG | 1 |
| NSP14 | Ser6061Ser | 0 | TCA | 3 | TCA | 3 | TCA | 3 |
| NSP14 | Leu6073Leu | 0 | TTA | 2 | TTA | 2 | TTA | 2 |
| NSP14 | Leu6076Leu | 0 | TTA | 2 | TTA | 2 | TTA | 2 |
| NSP14 | Leu6081Leu | 0 | TTA | 2 | TTA | 2 | TTA | 2 |
| NSP14 | Ser6095Ser | 0 | TCA | 3 | TCA | 3 | TCA | 3 |
| NSP14 | Leu6098Leu | 0 | TTA | 2 | TTA | 2 | TTA | 2 |
| NSP14 | Leu6101Leu | 0 | TTG | 2 | TTG | 2 | TTG | 2 |
| NSP14 | Ser6102Ser | 0 | TCA | 1 | TCA | 1 | TCA | 1 |
| NSP14 | Ser6118Ser | 0 | TCA | 1 | TCA | 1 | TCA | 1 |
| NSP14 | Leu6133Leu | 0 | TTA | 1 | TTA | 1 | TTA | 1 |
| NSP14 | Ser6142Ser | 0 | TCG | 1 | TCG | 1 | TCG | 1 |
| NSP14 | Ser6154Ser | 0 | TCA | 1 | TCA | 1 | TCA | 1 |
| NSP14 | Leu6177Leu | 0 | TTA | 1 | TTA | 1 | TTA | 1 |
| NSP14 | Ser6179Ser | 0 | TCG | 3 | TCG | 3 | TCG | 3 |
| NSP14 | Leu6183Leu | 0 | TTG | 1 | TTG | 1 | TTG | 1 |
| NSP14 | Ser6195Ser | 0 | TCA | 3 | TCA | 3 | TCA | 3 |
| NSP14 | Leu620462 | 0 | TTA | 1 | TTA | 1 | TTA | 1 |
| NSP14 | Leu6253Leu | 0 | TTA | 2 | TTA | 2 | TTA | 2 |
| NSP14 | Ser6281Ser | 0 | TCA | 3 | TCA | 3 | TCA | 3 |
| NSP14 | Ser6293Ser | 0 | TCA | 1 | TCA | 1 | TCA | 1 |
| NSP14 | Leu6307Leu | 0 | TTA | 1 | TTA | 1 | TTA | 1 |
| NSP14 | Ser6320Ser | 0 | TCG | 1 | TCG | 1 | TCG | 1 |
| NSP14 | Leu6330Leu | 0 | TTA | 1 | TTA | 1 | TTA | 1 |
| NSP14 | Ser6331Ser | 0 | TCA | 1 | TCA | 1 | TCA | 1 |
| NSP14 | Leu6333Leu | 0 | TTA | 2 | TTA | 2 | TTA | 2 |
| NSP14 | Ser6342Ser | 0 | TCA | 3 | TCA | 3 | TCA | 3 |
| NSP14 | Ser6358Ser | 0 | TCA | 3 | TCA | 3 | TCA | 3 |
| NSP14 | Ser6372Ser | 0 | TCA | 1 | TCA | 1 | TCA | 1 |
| NSP14 | Ser6374Ser | 0 | TCA | 3 | TCA | 3 | TCA | 3 |
| NSP14 | Ser6378Ser | 0 | TCA | 1 | TCA | 1 | TCA | 1 |
| NSP14 | Leu6392Leu | 0 | TTA | 1 | TTA | 1 | TTA | 1 |
| NSP14 | Ser6394Ser | 0 | TCA | 1 | TCA | 1 | TCA | 1 |
| NSP14 | Leu6419Leu | 0 | TTG | 2 | TTG | 2 | TTG | 2 |
| NSP14 | Ser6431Ser | 0 | TCG | 3 | TCG | 3 | TCG | 3 |
| NSP14 | Leu6443Leu | 0 | TTG | 2 | TTG | 2 | TTG | 2 |
| NSP14 | Leu6450Leu | 0 | TTA | 2 | TTA | 2 | TTA | 2 |
| NSP15 | Ser6452Ser | 0 | TCA | 3 | TCA | 3 | TCA | 3 |
| NSP15 | Ser6476Ser | 0 | TCA | 1 | TCA | 1 | TCA | 1 |
| NSP15 | Leu6508Leu | 0 | TTA | 2 | TTA | 2 | TTA | 2 |
| NSP15 | Leu6523Leu | 0 | TTG | 2 | TTG | 2 | TTG | 2 |
| NSP15 | Ser6548Ser | 0 | TCA | 3 | TCA | 3 | TCA | 3 |
| NSP15 | Ser6554Ser | 0 | TCA | 1 | TCA | 1 | TCA | 1 |
| NSP15 | Leu6570Leu | 0 | TTG | 2 | TTG | 2 | TTG | 2 |
| NSP15 | Leu6593Leu | 0 | TTA | 2 | TTA | 2 | TTA | 2 |
| NSP15 | Ser6598Ser | 0 | TCA | 3 | TCA | 3 | TCA | 3 |
| NSP15 | Ser6605Ser | 0 | TCA | 1 | TCA | 1 | TCA | 1 |
| NSP15 | Ser6612Ser | 0 | TCA | 3 | TCA | 3 | TCA | 3 |
| NSP15 | Leu6613Leu | 0 | TTA | 2 | TTA | 2 | TTA | 2 |

### Example 2

### Mutation of Nsp1

The inventors explored as a strategy for the development of a live-attenuated vaccine for SARS-CoV-2. The Nsp1 double mutant K164A/H165A loses its inhibition capability and the inventors' preliminary analysis of transcriptional responses to SARS-CoV-2 Nsp1 mutant infection confirms an increased host response to infection.

The inventors additionally mutated Nsp1 in two positions (K164A, H165A), and deleted accessory ORFs 6-8.

### Deletion of the FCS Region

The FCS region was deleted as described in Davidson A D, Williamson M K, Lewis S, et al., 2020, Genome Med. 2020; 12 (1): 68.

The inventors infected hamsters with the OTS viruses by intranasal administration of 5000 PFU/mouse, followed by a challenge infection with the ancestral SARS-CoV-2 (Wuhan wild-type (WT)) 21-days post-infection (FIG. 10).

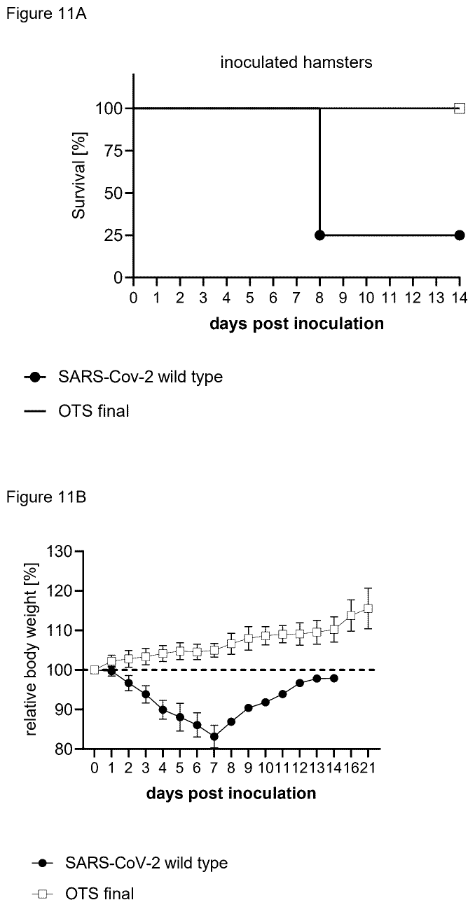

The inventors evaluated the survival of animals inoculated with OTS viruses or SARS-CoV-2 WT. (FIG. 11). 75% of the animals inoculated with SARS-CoV-2 wild-type succumbed to the disease or reached termination criteria within 8 days post-inoculation. In strong contrast, none of the animals inoculated with OTS constructs died.

Animals inoculated with SARS-CoV-2 WT, OTS4-5 and OTS7-8 viruses lost weight upon infection (mean bodyweights=84% (7 dpi), 91% (8 dpi) and 89% (7 dpi), respectively). In strong contrast, animals inoculated with OTS 4-5-6-7-8 Nsp1 ^{K164A/H165A}.deIORF6-8 and OTS 4-5-6-7-8 Nsp1 ^{K164A/H165A}.deIORF6-8.FCS (SEQ ID NO: 6 referred to as OTS final in the figures) gradually gained weight (mean bodyweight=106% (7 dpi) and 108% (8 dpi)), indicative of the lack of pathogenicity of OTS 4-5-6-7-8 Nsp1 ^{K164A/H165A}.deIORF6-8 and OTS 4-5-6-7-8 Nsp1 _{K164A/H165A}.deIORF6-8.FCS in the highly sensitive Syrian hamster model (FIG. 11).

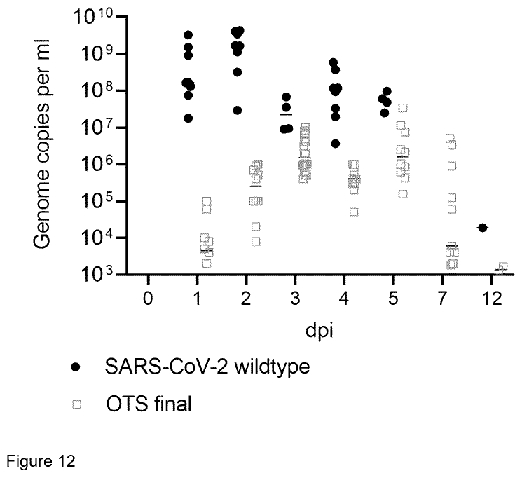

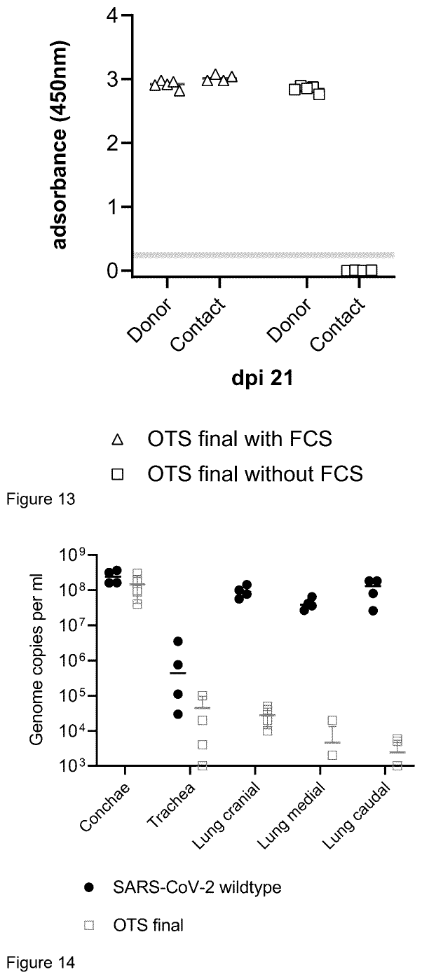

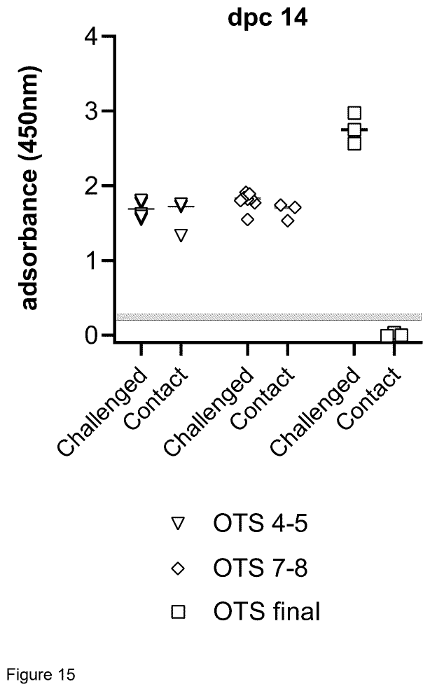

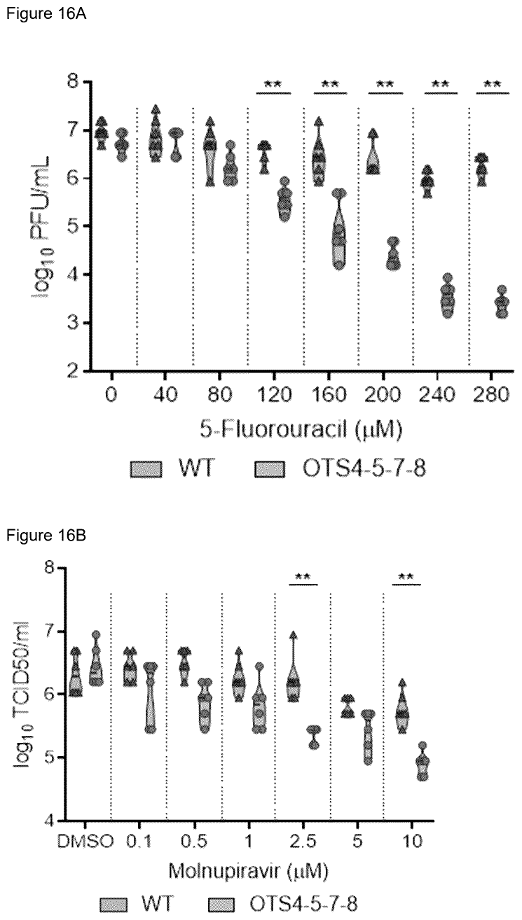

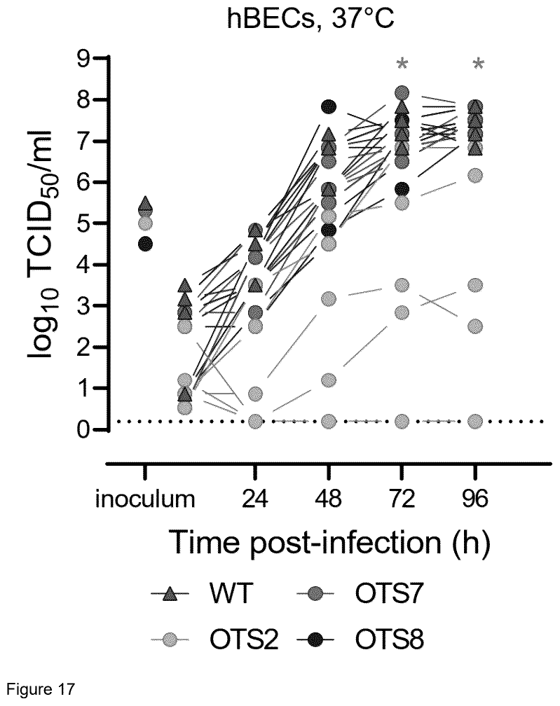

Additionally, conchae, trachea, lung (cranial, medial, caudal) samples, and nasal washing samples were collected 5 days post-infection and analyzed by an ORF1ab (Nsp12) specific RT-qPCR. By using a genome copy standard, the total amount of virus genome copies per ml (gc/ml) was calculated for each sample. Based on this information the amounts of virus genome copies were compared to each other and a fold change value was calculated (FIG. 12-14). Hamsters infected with SARS-CoV-2 WT, OTS4-5, and OTS7-8 did not differ in their virus genome loads in organs and washing samples. In contrast, OTS 4-5-6-7-8 Nsp1 ^{K164A/H165A}.deIORF6-8 and OTS 4-5-6-7-8 Nsp1 ^{K164A/H165A}.deIORF6-8.FCS had reduced virus genome load in the organs and in the washing samples.

The in vivo evaluation of OTS vaccine candidates OTS4-5, OTS7-8, and OTS 4-5-6-7-8 Nsp1 ^{K164A/H165A}.deIORF6-8 and OTS 4-5-6-7-8 Nsp1 ^{K164A/H165A}.deIORF6-8.FCS in Syrian hamsters confirms the partial attenuation of OTS4-5 and OTS7-8 and the improved properties of OTS 4-5-6-7-8 Nsp1 ^{K164A/H165A}.deIORF6-8 and OTS 4-5-6-7-8 Nap1 ^{K164A/H165A}.deIORF6-8.FCS.

### Example 3

Adding a mutagen such as 5-Fluorouracil or Malnupiravir reduces the number of infectious virus particles in a TCID50 virus assay. Particularly, the OTS virus is more prone to inactivation by a mutagen than WT SARS-CoV-2.

1. A polynucleotide encoding an attenuated human coronavirus or a fragment thereof, wherein the polynucleotide comprises at least 20 one-to-stop codons, wherein a one-to-stop codon is
i) a different but synonymous codon compared to a corresponding codon in a natural human coronavirus genome or a fragment thereof; and
ii) differs by only one nucleotide from a STOP codon.

2. The polynucleotide of claim 1, wherein the fragment of the polynucleotide when combined with corresponding human coronavirus parts encodes a coronavirus particle that induces an immune response after immunization of mice with 5000 PFU coronavirus particle after 15 days and an increased immune response upon challenge with WT human coronavirus after 21 days measured after 35 days.

3. A method for producing the polynucleotide of claim 1, the method comprising the steps of:
a) providing the CDS of a natural human coronavirus genome, a fragment or cDNA clone thereof; and
b) modifying the natural human coronavirus genome, the fragment or the retro-transcribed cDNA sequence of the cDNA clone, respectively,
wherein said modification comprises replacing at least 20 codons in the natural human coronavirus genome, the fragment or the retro-transcribed cDNA sequence, by at least 20 one-to-stop codons, wherein a one-to-stop codon is
i) a different but synonymous codon compared to a corresponding codon in the natural human coronavirus genome, the fragment or the retro-transcribed cDNA sequence; and
ii) differs by only one nucleotide from a STOP codon.

4. The polynucleotide of claim 1, wherein the natural human coronavirus genome or a fragment thereof is
a) a SARS-CoV-2 sequence comprised in or consisting of a sequence as defined by SEQ ID NO: 7 or
b) a SARS-CoV-2 sequence being 80% identical to a sequence comprised in or consisting of sequence as defined by SEQ ID NO: 7, preferably a SARS-CoV-2 sequence being 80% identical to a sequence comprised in or consisting of sequence as defined by SEQ ID NO: 7 which maintains the ability to encode one or more SARS-CoV-2 virus proteins.

5. The polynucleotide of claim 1, wherein the fragment has a minimum length of 500 nucleotides.

6. The polynucleotide of claim 1, wherein the human coronavirus is SARS-CoV-2 and wherein at least one of the one-to-stop codons is comprised in a sequence part or fragment corresponding to a sequence part of ORF1ab of the natural SARS-CoV-2, a sequence part encoding a structure protein of the natural SARS-CoV-2 or a sequence part encoding an accessory protein of the natural SARS-CoV-2, optionally wherein:
at least one of the one-to-stop codons is comprised in a sequence part or fragment corresponding to a sequence part of ORF1ab of the natural SARS-CoV-2;
at least one of the one-to-stop codons is comprised in a sequence part or fragment corresponding to an Nsp2 to Nsp15 encoding sequence part of the natural SARS-CoV-2 genome;
at least one of the one-to-stop codons is comprised in a sequence part or fragment corresponding to an Nsp2 to Nsp7 or an Nsp13 to Nsp15 encoding sequence part of the natural SARS-CoV-2 genome; and/or
the one-to-stop codons comprise at least one one-to-stop codon having a position selected from Table 1 corresponding to a position on the natural SARS-CoV-2 genome.

7-10. (canceled)

11. The polynucleotide of claim 1, wherein the amino acids encoded by the at least 20 one-to-stop codons consist of Leu, Ser, Arg and/or Gly, optionally wherein the amino acids encoded by the one-to-stop codons consist of Leu and/or Ser.

12. (canceled)

13. The polynucleotide of claim 1, wherein the at least 20 one-to-stop codons are at least 50 one-to-stop codons.

14. The polynucleotide of claim 1, wherein the human coronavirus is SARS-CoV-2 and wherein the polynucleotide comprises no sequence encoding a protein having an Nsp1 functionality of the natural SARS-CoV-2 or a sequence encoding a protein having a reduced Nsp1 functionality compared to the Nsp1 of a natural SARS-CoV-2, preferably wherein the polynucleotide comprises a sequence encoding a protein having a reduced Nsp1 functionality compared to the Nsp1 of a natural SARS-CoV-2, and polynucleotide comprises a mutation compared to the Nsp1 encoding sequence of natural SARS-CoV-2, wherein the mutation is K164A and/or H165A.

15. The polynucleotide of claim 1, wherein the human coronavirus is SARS-CoV-2 and wherein the polynucleotide comprises no sequence encoding a protein having the functionality of a protein encoded by the ORF6 gene of the natural SARS-CoV-2 or a sequence encoding a protein having a reduced functionality of a protein encoded by the ORF6 gene of the natural SARS-CoV-2.

16. The polynucleotide of claim 1, wherein the human coronavirus is SARS-CoV-2 and wherein the polynucleotide comprises no sequence encoding a protein having the functionality of a protein encoded by the ORF7a gene of the natural SARS-CoV-2 or a sequence encoding a protein having a reduced functionality of a protein encoded by the ORF7a gene of the natural SARS-CoV-2.

17. The polynucleotide of claim 1, wherein the human coronavirus is SARS-CoV-2 and wherein the polynucleotide comprises no sequence encoding a protein having the functionality of a protein encoded by the ORF7b gene of the natural SARS-CoV-2 or a sequence encoding a protein having a reduced functionality of a protein encoded by the ORF7b gene of the natural SARS-CoV-2.

18. The polynucleotide of claim 1, wherein the human coronavirus is SARS-CoV-2 and wherein the polynucleotide comprises no sequence encoding a protein having the functionality of a protein encoded by the ORF8 gene of the natural SARS-CoV-2 or a sequence encoding a protein having a reduced functionality of a protein encoded by the ORF8 gene of the natural SARS-CoV-2.

19. The polynucleotide of claim 1, wherein the human coronavirus is SARS-CoV-2 and wherein the polynucleotide comprises a sequence part encoding a spike protein, wherein the spike protein comprises a modified or removed cleavage site compared to the cleavage site of the spike protein of the natural SARS-CoV-2, optionally wherein the polynucleotide consists of or comprises a sequence as defined SEQ ID NO: 6.

20. (canceled)

21. A vector comprising the polynucleotide of, claim 1.

22. A genetically modified cell comprising the polynucleotide of claim 1.

23. A method for production of an attenuated virus, the method comprising a step of culturing the genetically modified cell of claim 22.

24. An attenuated virus comprising the polynucleotide of claim 1.

25-27. (canceled)

28. A method of treatment and/or prevention comprising the step of:
Administering a pharmaceutical product in a therapeutically effective amount to a subject, wherein the pharmaceutical product comprises the vector of claim 21, optionally wherein:
the treatment and/or prevention is a treatment and/or prevention of a human coronavirus infection, preferably a SARS-CoV-2 infection;
the method further comprises administering a mutagen in a therapeutically effective amount to a subject, optionally wherein the mutagen is 5-Fluorouracil or Malnupiravir.

29-31. (canceled)
